(19) United States
(12) Patent Application Publication
(10) Pub. No.: US 2004/0157280 A1
(43) Pub. Date: Aug. 12, 2004

(54) Antibody mediated ozone generation

(76) Inventors: Paul Wentworth, San Diego, CA (US); Richard A. Lerner, La Jolla, CA (US)

Correspondence Address: Schwegman, Lundberg, Woessner & Kluth, P.A., P.O. Box 2938, Minneapolis, MN, 55402, US

(21) Appl. No.: 10/714,567

(22) Filed: Nov. 14, 2003

Related U.S. Application Data
(60) Provisional application No. 60/426,245, filed on Nov. 14, 2002.

(30) Foreign Application Priority Data
Sep. 17, 2001 (WO) PCT/US01/29165

(51) Int. Cl. A61K 33/00; G01N 33/53; G01N 33/567; G01N 33/537; G01N 33/543

(52) U.S. Cl. 435/7.92; 424/600

(57) ABSTRACT

The invention provides methods of detecting antibodies and neutrophils that can generate reactive oxygen species.

### RELATED APPLICATIONS

This application claims priority under 35 U.S.C. 119(e) to U.S. Provisional Application Ser. No. 60/426,245 filed Nov. 14, 2002, which is in corporated herein by reference.

This application also claims priority from U.S. application Ser. No. 10/380,905 filed Mar. 17, 2003, which is a U.S. National Stage filing from International Application Ser. No. PCT Application No. PCT/US01/29165 filed Sep. 17, 2001 and published in English as WO 02/022573 on Mar. 21, 2002, which claimed priority from U.S. Provisional Application Ser. No. 60/315,906 filed Aug. 29, 2001, U.S. Provisional Application Ser. No. 60/235,475 filed Sep. 26, 2000, and U.S. Provisional Application Ser. No. 60/232,702 filed Sep. 15, 2001, which applications.

This application is also related to Provisional Application Ser. No. 60/426,242 filed Nov. 14, 2002 and to U.S. application Ser. No. ______ (Atty. Docket No. 1361.027US 1) filed on even date herewith.

### GOVERNMENT SUPPORT

Work contributing to this invention was supported by a grant from the National Institutes of Health, GM43858, POCA277489. Accordingly, the United States government may have certain rights in the invention.

### FIELD OF THE INVENTION

The present invention relates generally to the field of detecting immunological and inflammatory reactions in vivo or in vitro by detection of antibody-mediated or neutrophil-mediated generation of reactive oxygen species. The invention also provides methods for detecting neutrophil activation by detecting neutrophil-mediated generation of reactive oxygen species. The invention also relates to methods for identifying agents that can modulate an immune response or modulate neutrophil activation.

### BACKGROUND

Research throughout the last century has led to a consensus as to the role of antibodies in the immune system. The essence of this consensus is that the antibody molecule does not generate any detectable products. Instead, the antibody molecule has been perceived as a binding molecule that merely tags its target or that activates other molecules or biological systems to respond to antibody-antigen union. Hence, antibodies themselves have been perceived as not possessing any catalytic activities but as only marking foreign substances for removal by the complement cascade and/or phagocytosis (Arlaud et al., Immunol. Today, 8, 106-111 (1987); Sim & Reid, Immunol. Today, 12, 307-311 (1991)).

Moreover, although the neutrophil inflammatory response is essential for the destruction of bacteria that invade the body, inappropriate neutrophil activation can cause several problems. For example, if neutrophils are properly primed when attracted to the lungs, they can release destructive enzymes into the lung tissue. This can lead to the development of adult respiratory distress syndrome (ARDS) (Weiland et al., Amer. Rev. Respir. Dis., 133:218-225, 1986; Idell et al, Am. Rev. Respir. Dis., 132:1098-1105, 1985). ARDS attacks between 150,000 and 200,000 Americans per year, with a mortality rate of 50-80% in even the best clinical facilities (Balk and Bone, 1983). ARDS is initiated by bacterial infections, sudden severe dropping of the blood pressure (shock), and many other insults to the body.

Accordingly, improved methods are needed so that neutrophil activation, inflammation and other immune responses can be quickly and effectively detected.

### SUMMARY OF THE INVENTION

The invention provides methods for utilizing the newly discovered abilities of antibodies and neutrophils to reduce singlet oxygen to reactive oxygen species. According to the invention, antibodies and neutrophils can generate ozone (O₃) and other reactive oxygen species when exposed to singlet oxygen (¹O₂*). Antibodies perform such conversion without the need for any other component of the immune system, that is, without the need for the complement cascade or phagocytosis. Moreover, according to the invention, ozone is also produced by antibody-coated mammalian leukocytes such as neutrophils.

The invention therefore provides improved assays based on the direct detection of reactive oxygen species that are produced by antibody-catalyzed and neutrophil-catalyzed reactions.

In one embodiment, the invention provides a method for assaying for an immunological response or for an inflammatory response in a mammal comprising: (a) administering a suitable chemical probe for a reactive oxygen species; (b) obtaining a sample from the mammal; and (c) analyzing the sample for oxidation products of the chemical probe.

In another embodiment, the invention provides an in vitro assay for neutrophil activity comprising: (a) obtaining a neutrophil sample from a mammal; (b) activating neutrophils in the neutrophil sample; and (c) observing whether a reactive oxygen species can be detected in the neutrophil sample.

In yet another embodiment, the invention provides a method for identifying an agent that can modulate neutrophil activity comprising: (a) obtaining a neutrophil sample from a mammal; (b) exposing the neutrophil sample to a test agent; (c) activating neutrophils in the neutrophil sample; and (d) quantifying the amount of reactive oxygen species generated by the neutrophil sample.

Reactive oxygen species that can be detected include any antibody or neutrophil generated reactive oxygen species. Examples include, but are not limited to, superoxide radical (O₂?), hydroxyl radical (OH^{?}), peroxyl radical, hydrogen peroxide (H₂O₂) or ozone (O₃). The presence of such powerful reactive oxygen species is indicative of an increased humoral immune response (e.g. increased circulating antibodies) or an increased cellular or tissue related inflammatory response (e.g. neutrophil activation).

### BRIEF DESCRIPTION OF THE DRAWINGS

FIG. 1 illustrates the oxygen-dependent microbicidal action of phagocytes. The interconversion of ¹O₂ and O₂^{?}?is indicated.

FIG. 2 illustrates the chemical conversion steps involved in the amplex red assay. An antibody (identified as IgG in this schematic drawing) converts ¹O₂ to O₂^{?}?, which can spontaneously form hydrogen peroxide. In the presence of horseradish peroxidase, the hydrogen peroxide deacetylates and oxidizes the amplex red substrate, thereby generating molecule that emits fluorescence at 587 nm.

FIG. 3 shows the initial time course of H₂O₂ production in PBS (pH 7.4) in the presence (?) or absence (?) of murine monoclonal IgG EP2-19G2 (20 μM). Error bars show the range of the data from the mean.

FIG. 4 shows the fluorescent micrograph of a single crystal of murine antibody 1D4 Fab fragment after UV irradiation and H₂O₂ detection with the amplex red reagent.

FIGS. 5A-D illustrate the time course and reaction conditions required for antibody-mediated catalysis of reactive oxygen species. FIG. 5A provides a time course of H₂O₂ formation in PBS (pH 7.4) with hematoporphyrin (40 μM) and visible light, in the presence (?, filled circles) or absence (?, filled diamonds) of 31127 antibody (horse IgG, 20 μM). FIG. 5B provides an initial time course of H₂O₂ production with hematoporphyrin (40 μM) and visible light in the presence of 31127 antibody (horse IgG, 6.7 μM) with no additive in PBS (pH 7.4) (¦, filled squares) or NaN₃ in PBS (pH 7.4) (?, filled circles, 100 μM) or in a D₂O solution of PBS (pH 7.4) (?, filled diamonds). FIG. 5C illustrates the effect of antibody protein concentration (31127, horse IgG) on the rate of H₂O₂ formation. FIG. 5D illustrates the effect of oxygen concentration on the rate of H₂O₂ generation by the 31127 antibody (horse IgG, 6.7 μM). All points are mean values of at least duplicate experimental determinations. Error bars are the range of experimentally measured values from the mean.

FIG. 6 is a bar graph showing the measured initial rate of H₂O₂ formation for a panel of proteins and comparison with antibodies (data from Table 1). All points are mean values of at least duplicate experimental determinations. Error bars are the range of experimentally measured values from the mean. OVA, chick-egg ovalbumin; SOD, superoxide dismutase.

FIG. 7A illustrates the rate of H₂O₂ formation by UV irradiation of horse IgG (6.7 μM) in PBS (pH 7.4). FIG. 7B illustrates the fluorescence emission at 326 nm (excitation=280 nm) of the horse IgG, measured simultaneously with H₂O₂ formation.

FIGS. 8A-F illustrate H₂O₂ production by antibodies under various conditions.

FIG. 8A illustrates the production of H₂O₂ by immunoglobulins and non-immunoglobulin proteins. Assays were performed by near-UV irradiation (312 nm, 800 μW cm⁻²) of individual antibody/protein samples (100 μL, 6.7 μM) in phosphate-buffered saline (PBS) [10 mM sodium phosphate, 150 mM NaCl (pH 7.4)] in a sealed glass vial on a transilluminator (Fischer Biotech) under ambient aerobic conditions at 20EC. Aliquots (10 μL) were removed at timed intervals throughout the assay. H₂O₂ concentration was determined by the amplex red method. Each data point is reported as the mean±SEM of at least duplicate measurements: ? polyclonal (poly) immunoglobulin (Ig) G, human; O poly-IgG, horse; ? poly-IgG, sheep; ∇ monoclonal (m) IgG (WD1-6G6), murine; ? poly-IgM, human; ? mIgG (92H2), murine; ¦ β-galactosidase (β-gal); ? chick ovalbumin (OVA); ? a-lactalbumin (a-lact); ? bovine serum albumin (BSA).

FIG. 8B illustrates the long-term production of H₂O₂ by sheep poly-IgG (6.7 μM, 200 μL). Near-UV irradiation for 8 hours in PBS in a sealed well of a 96-well quartz plate. H₂O₂ concentration was measured as described in FIG. 8A. FIG. 8C illustrates the effect of catalase on the antibody-catalyzed production of H₂O₂ over time. A solution of murine monoclonal antibody PCP-21H3 (IgG) (6.7 μM, 200 μL), was irradiated in PBS in a sealed well of a 96 well quartz plate for 510 min. The H₂O₂ was assayed by the amplex red assay and then destroyed by addition of catalase (10 mg, 288 mU) immobilized on Eupergit C. The catalase was removed by filtration and the antibody solution re-irradiated for 420 min. Rate (0-510 min)=0.368, μM min⁻¹ (r²=0.998); rate (511-930 min)=0.398 μM min⁻¹ (r²=0.987).

FIG. 8D illustrates the effect of H₂O₂ concentration on the percent maximum rate of catalysis by horse poly-IgG antibody. Such a graph permits determination of the IC₅₀ of H₂O₂ on the photo-production of H₂O₂ by horse poly-IgG. A solution of horse IgG (6.7 μM) was incubated with varying concentrations of H₂O₂ (0-450 μM) and the initial rate of H₂O₂ formation measured as described in FIG. 8A. The graph is a plot of rate of H₂O₂ formation Versus H₂O₂ concentration and reveals an IC₅₀ of 225 μM.

FIG. 8E illustrates the long-term inhibition of antibody photo-production of H₂O₂ by H₂O₂ and complete re-establishment of activity after removal of H₂O₂. The assay involved an initial U.V. irradiation of horse poly-IgG (6.7 mM in PBS pH 7.4) in the presence of H₂O₂ (450 μM) for 360 min. The H₂O₂ was then removed by catalase (immobilized on Eupergit C) and the poly-IgG sample was re-irradiated with UV light for a further 480 minutes. H₂O₂ fo rmation throughout the assay was measured by the amplex red assay.

FIG. 8F illustrates the effect of catalase on H₂O₂ production. A solution of aβ-TCR (6.7 μM, 200 μL) was irradiated as described for FIG. 8C for periods of 360, 367 and 389 min. The H₂O₂ generated during each irradiation was assayed and destroyed as described for FIG. 8C. Rate (0-360 min)=0.693 μM min⁻¹(r²=0.962). The curvature in the progress curve above 200 μM conforms to the expected inhibition by H₂O₂ (vide infra); rate (361-727 min)=0.427 μM min⁻¹ (r²=0.987); rate (728-1117 min)=0.386 μM min⁻¹ (r²=0.991).

FIGS. 9A-B illustrate the superposition of native 4C6 Fab (light blue and pink in a color photograph) and 4C6 Fab in the presence of H₂O₂ (dark blue and red in a color photograph).

For FIG. 9A, the native 4C6 crystals were soaked for 3 minutes in 4 mM H₂O₂, and immediately flash frozen for data collection at SSRL BL 9-1. The overall structural integrity of the secondary and tertiary structure is clearly preserved in the presence of H₂O₂ (RMSD Ca=0.33 Å, side chain =0.49 Å). The RMSD was calculated in CNS.

FIG. 9B illustrates the binding of benzoic to Fab 4C6. High resolution x-ray structures show that Fab 4C6 is cross-reactive with benzoic acid. Superposition of the 4C6 combining site with and without H₂O₂ demonstrates that even the side chain conformations within the binding site are preserved (light and dark colored side chains in a color photograph correspond to + and −H₂O₂ respectively). Moreover, clear electron density for the benzoic acid underscores that the binding properties of Fab 4C6 remain unaltered in 4 mM H₂O₂. The electron density map is a 2fₒ-f_{c} sigma weighted map contoured at 1.5s, and the figures were generated in Bobscript.

FIG. 10A shows the absorbance spectra of horse polyclonal IgG measured on a diode array HP8452A spectrophotometer, Absₘₐₓ 280 nm.

FIG. 10B provides an action spectra of horse polyclonal IgG, between wavelengths 260 and 320 nm showing maximum activity of H₂O₂ formation at 280 nm. The assay was performed in duplicate and involved addition of an antibody solution [6.7 μM in PBS (pH 7.4)] to a quartz tube that was then placed in a light beam produced by a xenon arc lamp and monochromator of an SLM spectrofluorimeter for 1 hour. H₂O₂ concentration was measured by the amplex red assay.

FIG. 11A illustrates the production of H₂O₂ over time by tryptophan (20 μM). The conditions and assay procedures were as described in FIG. 8A.

FIG. 11B provides the effect of chloride ion on antibody-mediated photo-production of H₂O₂. A solution of sheep poly-IgG: (6.7 μM, 200 μL) or horse poly-IgG ? (6.7 μM, 200 μL) was lyophilized to dryness and then dissolved in either deionized water or NaCl (aq.) such that the final concentration of chloride ion was 0-160 mM. The samples were then irradiated, in duplicate, in sealed glass vials on a transilluminator (800 μW cm⁻²) under ambient aerobic conditions at 20 EC. Aliquots (10 μL) were removed throughout the assay and the H₂O₂ concentration determined by the amplex red assay. The rate of H₂O₂ formation is plotted as the mean±S.E.M. versus [NaCl] for each antibody sample.

FIG. 11C illustrates the effect of dialysis in EDTA-containing buffers on antibody-mediated photo-production of H₂O₂. The photo-production of H₂O₂ by two antibody preparations, mouse monoclonal antibody PCP21H3 and horse polyclonal IgG, were compared before and after dialysis into PBS containing EDTA (20 mM). The conditions and assay procedures were as described in FIG. 8A. Each data point is reported as the mean±SEM of at least duplicate measurements: [? murine mIgG PCP21H3 before dialysis; ¦ murine mIgG PCP21H3 after dialysis; ? poly-IgG, horse before dialysis; ? poly-IgG, horse after dialysis.

FIGS. 12A-F provide mass spectra illustrating oxidation of the substrate tris carboxyethyl phosphine (TCEP) with either 160 containing H₂O₂ or with 180 containing H₂O₂. ESI (negative polarity) mass spectra were taken of TCEP [(M−H)-249] and its oxides [(M−H)⁻265 (¹⁶O) and (M−H)⁻267 (¹⁸O)] after oxidation with H₂O₂.

FIG. 12A provides the mass spectrum of TCEP and its oxides after irradiation of sheep poly-IgG (6.7/μM) under 1602 aerobic conditions in H₂¹⁸O (98% ¹⁸O) PB. A mix of ¹⁶O containing TCEP (larger peak at 265) and ¹⁸O containing TCEP (smaller peak at 267) is produced.

FIG. 12B provides the mass spectrum of TCEP and its oxides after irradiation of sheep poly-IgG (6.7 μM) under enriched ¹⁸O (90% ¹⁸O) aerobic conditions in H₂ ¹⁶O PB. A mix of ¹⁶O containing TCEP (smaller peak at 265) and ¹⁸O containing TCEP (larger peak at 267) is produced.

FIG. 12C provides the mass spectrum of TCEP and its oxides after irradiation of the poly-IgG performed under ¹⁶O₂ aerobic concentration in H₂¹⁶O PB. The assay conditions and procedures were as described in the methods and materials (Example II) with the exception that H₂¹⁶O replaced H₂¹⁸O. Only ¹⁶O containing TCEP (large peak at 265) is observed.

FIG. 12D provides the mass spectrum of TCEP and its oxides after irradiation of sheep poly-IgG (6.7 μM) and H₂¹⁶O₂ (200 μM) under anaerobic (degassed and under argon) conditions in H₂ 18O PB for 8 hours at 20EC. Addition of TCEP was as described in the methods and materials (Example II). Only ¹⁶O containing TCEP (large peak at 265) is observed.

FIG. 12E provides the mass spectrum of TCEP and its oxides after irradiation of 3-methylindole (500 μM) under ¹⁶O₂ aerobic conditions in H₂¹⁸O PB. Only ¹⁶O containing TCEP (large peak at 265) is observed. The assay conditions and procedures were as described in the methods and materials (Example II) with the exception that size-exclusion filtration was not performed because 3-methyl indole is of too low molecular weight. Therefore, TCEP was added to the 3-methyl indole-containing PB solution.

FIG. 12F provides the mass spectrum of TCEP and its oxides after irradiation of β-gal (50 μM) under ¹⁶O₂ aerobic conditions in H₂¹⁸O PB. Only ¹⁶O containing TCEP (large peak at 265) is observed. Assay conditions and procedures are as described in the methods and materials (Example II).

FIGS. 13A-B show the Xe binding sites in antibody 4C6 as described in materials and methods (Example II).

FIG. 13A provides a standard side view of the Ca trace of Fab 4C6 with the light chain in pink and the heavy chain in blue in a color photograph. Three bound xenon atoms (green in a color photograph) are shown with the initial Fₒ-F_{c} electron density map contoured at 5 s.

FIG. 13B provides an overlay of Fab 4C6 and the 2C aβ TCR (PDB/TCR) around the conserved xenon site 1. The backbone Cₐ trace of V_{L} (pink in a color photograph) and side chains (yellow in a color photograph) and the corresponding Vₐ of the 2C aβ TCR (red and gold in a color photograph) are superimposed (FIG. generated using Insight2000).

FIGS. 14A-D illustrate the killing of bacteria by antibodies.

FIG. 14A provides a bar-graph showing the survival of E. coli XL1-blue and O112a,c strains under different experimental conditions. Survival is reported as recovered colony forming units (CFUs) as a percent of the CFUs at the start of the experiment (t=0 min). Hatched bars and open bars correspond to the same experimental conditions except that the open bar groups (2, 4, 6, 8, 10 and 12) were exposed to visible light (2.7 mWcm⁻²) for 60 min, whereas the hatched bar groups (1, 3, 5, 7, 9 and 11) were placed in the dark for 60 min. The bacterial cell density was about 10⁷ cells/mL. Each data point reported is the mean±S.E.M. (n=6) of E. coli XL1-blue (groups 1-6) and O112 a,c (groups 7-12) under the following conditions. Groups 1-2 XL1-blue cells in PBS, pH 7.4 at 4° C. Groups 3-4 HPIX (40 μM), XL1-blue cells in PBS, pH 7.4 at 4° C. Groups 5-6 XL1-blue-specific monoclonal antibody (25D11, 20 μM), hematoporphyrin IX (40 μM), XL1-blue cells in PBS, pH 7.4 at 4° C. Groups 7-8 O112a,c cells in PBS, pH 7.4 at 4° C. Groups 9-10 HPIX (40 μM), O112a,c cells in PBS, pH 7.4 at 4° C. Groups 11-12 O112a,c-specific monoclonal antibody (15404, 20 μM), hematoporphyrin IX (40 μM), O112a,c cells in PBS, pH 7.4 at 4° C.

FIG. 14B graphically illustrates the effect of antibody concentration on the survival of E. coli O112a,c. The antibody employed was an O112a,c-specific monoclonal antibody, 15404. Each data point reported is the mean value±S.E.M (n=3). The concentration of 15404 antibody that corresponds to killing of 50% of the cells (EC₅₀) was 81±6 nM.

FIG. 14C graphically illustrates the effect of irradiation time on the bactericidal action of E. coli XL1-blue-specific murine monoclonal antibody 12B2. The graph provides irradiation time (2.7 mW cm⁻²) versus survival of E. coli XL1-blue in the presence of hematoporphyrin IX (40 μM) and 12B2 (20 μM). Each data point reported is the mean value±S.E.M (n=3). The time of irradiation that corresponds to killing of 50% of the cells was 30±2 min.

FIG. 14D illustrates the dependence of antibody driven bactericidal action on hematoporphyrin IX concentration. The antibody employed was the E. coli XL1-blue-specific murine monoclonal antibody 25D11. The graph provides survival of E. coli XL1-blue versus exposure to a range of hematoporphyrin IX concentrations. The following conditions were employed: XL1-blue cells in PBS, pH 7.4 at 4° C. in the dark, 60 min (▴). XL1-blue cells in PBS, pH 7.4 at 4° C. in white light (2.7 mW cm⁻²) (Δ). 25D11 (20 μM), XL1-blue cells in PBS, pH 7.4 at 4° C. in the dark, 60 min (♦). 25D11 (20 μM), XL1-blue cells in PBS, pH 7.4 at 4° C. in white light (2.7 mW cm⁻²) for 60 min (⋄).

FIG. 15 provides an electron micrograph of an E. coli O112a,c cell after exposure to antigen-specific murine monoclonal IgG (15404, 20 μM), hematoporphyrin IX (40 μM) in PBS and visible light for 1 h at 4° C. (<5% viable). To visualize the sites of antibody attachment gold-labeled goat anti-mouse antibodies were added after completion of the bactericidal assay. The potency of the bactericidal activity of antigen non-specific antibodies was observed to be very similar to antigen-specific antibodies. Typically 20 μM of antibody (non-specific) was >95% bactericidal in the assay system.

FIGS. 16A-C provide electron micrographs of E. coli XL-1 blue cells after exposure to non-specific murine monoclonal IgG antibodies (84G3, 20 μM), hematoporphyrin IX (40 μM) in PBS and visible light for 1 h at 4° C. (1% viable). The arrows in FIG. 16A point toward the preliminary separation of the cell membrane from the cytoplasmic contents. FIG. 16D provides an electron micrograph of serotype E. coli O112a,c after exposure to antigen-specific murine monoclonal IgG (15404, 10 μM), hematoporphyrin IX (40 μM) in PBS and visible light for 1 h at room temperature (<5% viable). Gold-labeling was performed using procedures available in the art. FIG. 17A illustrates the effect of catalase on the bactericidal action of antibodies against E. coli XL1-blue [reported as recovered colony forming units (CFUs) as a percent of the CFUs at the start of the experiment (t=0 min)]. Catalase converts H₂O₂ to water (H₂O) and molecular oxygen (O₂). Each group was irradiated with white light (2.7 mW cm⁻²) for 60 min at 4° C. The bacterial cell density was ˜10⁷ cells/mL. The experimental groups (1-7) were treated as follows: Group 1 E. coli XL1-blue cells and hematoporphyrin IX (40 μM) in PBS (pH 7.4). Group 2 E. coli XL1-blue cells and non-specific murine monoclonal antibody 84G3 (20 μM) in PBS (pH 7.4). Group 3 E. coli XL1-blue cells, hematoporphyrin IX (40 μM) and monoclonal antibody 84G3 (20 μM) in PBS (pH 7.4). Group 4 E. coli XL1-blue cells, hematoporphyrin IX (40 μM), monoclonal antibody 84G3 (20 μM) and catalase (13mU/mL) in PBS (pH 7.4). Group 5 E. coli XL1-blue cells and specific rabbit polyclonal antibody (20 μM) in PBS (pH 7.4). Group 6 E. coli XL1-blue cells, hematoporphyrin IX (40 μM) and specific rabbit polyclonal antibody (20 μM) in PBS (pH 7.4). Group 7 E. coli XL1-blue cells, hematoporphyrin IX (40 μM), specific rabbit polyclonal antibody (20 μM) and catalase (13 mU/mL) in PBS (pH 7.4). Each point is reported as the mean value±S.E.M. of multiple experiments (n=6). The symbol ** denotes a p value of <0.01 relative to controls at the same time point. No bactericidal activity was observed in any of the dark controls (data not shown).

FIG. 17B illustrates the concentration dependent toxicity of H₂O₂ on the viability of E. coli XL1-blue (¦) and O112a,c (?) serotypes. The vertical hatched line is the concentration of H₂O₂ expected to be generated by antibodies during a 60 min incubation using the conditions described above for FIG. 14 and in Hofinan et al., Infect. Immun. 68, 449 (2000). The value of 35±5 μM H₂O₂ is the mean value determined from at least duplicate assays of twelve different monoclonal antibodies.

FIG. 18 illustrates the progress of photo-production of isatin sulfonic acid 2 from indigo carmine 1 (1 mM) during u.v. irradiation (312 nm, 0.8 mW cm⁻²) of antibodies in PBS (pH 7.4) in the presence and absence of catalase. Steinbeck et al., J. Biol. Chem. 267, 13425 (1992). Each point is reported as the mean±S.E.M. of at least duplicate determinations. Linear regression analysis was performed with Graphpad Prism v.3.0 software. The rate of formation of isatin sulfonic acid 2 (ν) was observed under the following conditions: Sheep polyclonal IgG (20 μM)(•) ν=34.8±1.8 nM/min; Murine monoclonal antibody 33F12 (20 μM)( ) ν=40.5±1.5 nm/min; Sheep polyclonal IgG (20 μM) and soluble catalase (13 mU/mL)(Δ) ν=33.5 +2.3 nM/min; Murine monoclonal antibody 33F12 (20 μM) and soluble catalase (13 mU/mL)(∇) ν=41.8±1.2 nM/min.

FIGS. 19A-C provides electrospray ionization (negative polarity) mass spectra of isatin sulfonic acid 2 [(MH)-226, (M−H)-228 (¹⁸O) and (M−H)-(2×¹⁸O)] produced during the oxidation of indigo carmine 1 (1 mM) in H₂¹⁸O (>95% ¹⁸O) phosphate buffer (PB, 100 mM, pH 7.4) at room temperature under various conditions. FIG. 19A provides the mass spectrum of isatin sulfonic acid 2 produced during the oxidation of indigo carmine 1 by chemical ozonolysis (600 μM in PB) for 5 min. The ¹⁸O is the dark circle and the ¹⁶O is the open circle. FIG. 19B provides the mass spectrum of isatin sulfonic acid 2 produced during the oxidation of indigo carmine 1 by irradiation with white light (2.7 mW cm⁻²), hematoporphyrin IX (40 μM) and sheep poly-IgG (20 μM) for 4 h. FIG. 19C provides the mass spectrum of isatin sulfonic acid 2 produced during the oxidation of indigo carmine 1 by irradiation of hematoporphyrin IX (40 μM) with white light (2.7 mW cm⁻²) for 4 h.

FIG. 20A illustrates the time course of oxidation of indigo carmine 1 (30 μM) (Δ) and formation of 2 (▪) by human neutrophils (PMNs, 1.5×10⁷ cell/mL) activated with phorbol myristate (1 μg/mL) in PBS (pH 7.4) at 37° C. No oxidation of indigo carmine 1 occurs with PMNs that are not activated (data not shown). Neutrophils were prepared as previously described. Hypochlorous acid (HOCl) is an oxidant known to be produced by neutrophils. In our hands, NaOCl (2 mM) in PBS (pH 7.4) oxidizes 1 (100 μM) but does not cleave the double bond of 1 to yield isatin sulfonic acid 2.

FIG. 20B illustrates the negative-ion electrospray mass spectrum of the isatin sulfonic acid 2 produced during the oxidation of indigo carmine 1 by activated human neutrophils, under the conditions described in FIG. 20A.

### DETAILED DESCRIPTION OF THE INVENTION

The present invention concerns the discovery that antibodies and neutrophils have the ability to intercept singlet oxygen and convert it to reactive oxygen species. According to the invention, such reactive oxygen species are indicators of immunological activity, inflammation, or neutrophil activation. Examples of reactive oxygen species generated by antibodies and neutrophils include, but are not limited to, ozone (O₃), superoxide radical (O₂?), hydrogen peroxide (H₂O₂) or hydroxyl radical (OH^{?}).

The ability of antibodies and neutrophils to convert singlet oxygen to reactive oxygen species provides a means for detecting immunological activity, inflammation, or neutrophil activation. Accordingly, the invention provides a variety of in vitro or in vivo methods for detecting immunological activity, inflammation or neutrophil activation. Also contemplated, are methods for identifying factors that can modulate the immune system and/or neutrophil activation.

Definitions

Abbreviations: (HP) hematoporphyrin; (PBS) phosphate buffered saline; (OVA) chick-egg ovalbumin; (SOD) superoxide dismutase; (PO) peroxidase enzymes; (phox) phagocyte oxidase; (HRP) horseradish peroxidase; (MS) mass spectroscopy; (AES) ICP-atomic emission spectroscopy; (MS) mass-spectral, (QC) quantum chemical.

The term “agent” herein is used to denotes a chemical compound, a mixture of chemical compounds, a biological macromolecule, or an extract made from biological materials such as bacteria, plants, fungi, or animal (particularly mammalian) cells or tissues. Agents are evaluated for potential activity as antibody or neutrophil modulatory agents by screening assays described herein.

The terms “effective amount,” “effective reducing amount,” “effective ameliorating amount”, “effective tissue injury inhibiting amount”, “therapeutically effective amount” and the like terms as used herein are terms to identify an amount sufficient to obtain the desired physiological effect, e.g., treatment of a condition, disorder, disease and the like or reduction in symptoms of the condition, disorder, disease and the like. An effective amount of a neutrophil modulating agent in the context of therapeutic methods is an amount that results in reducing, reversing, ameliorating, or inhibiting an inappropriate neutrophil response.

An “engineered antibody molecule” is a polypeptide that has been produced through recombinant techniques. Such molecules can include a reactive center that can catalyze the production of at least one reactive oxygen species from singlet oxygen. Such engineered antibody molecules may have a reactive indole contained within a polypeptide structure. The indole of such a molecule may be present as a tryptophan residue. Engineered antibody molecules may also contain non-natural amino acids and linkages as well as peptidomimetics. Engineered antibody molecules also include antibodies that are modified to eliminate the reaction center such that they are substantially unable to generate reactive oxygen species.

As used herein, the term “epitope” means any antigenic determinant on an antigen to which the paratope of an antibody binds. Epitopic determinants usually consist of chemically active surface groupings of molecules such as amino acids or sugar side chains and usually have specific three-dimensional structural characteristics, as well as specific charge characteristics. Antigens can include polypeptides, fatty acids, lipoproteins, lipids, chemicals, hormones and the like. In some embodiments, antigens include, but are not limited to, proteins from microbes such as bacteria or viruses such as human immunodeficiency virus, influenza virus, herpesvirus, papillomavirus, human T-cell leukemia virus and the like. In other embodiments, antigens include, but are not limited to, proteins expressed on cancer cells such as lung cancer, prostate cancer, colon cancer, cervical cancer, endometrial cancer, bladder cancer, bone cancer, leukemia, lymphoma, brain cancer and the like. Antigens of the invention also include chemicals such as ethanol, tetrahydrocanabinol, LSD, heroin, cocaine and the like.

The term “modulate” refers to the capacity to either enhance or inhibit a functional property of an antibody, neutrophil or engineered antibody molecule of the invention. Such modulation may increase or decrease production of at least one reactive oxygen species by the antibody, neutrophil or engineered antibody molecule. Such modulation may also increase or decrease neutrophil activation.

A “non-natural” amino acid includes D-amino acids as well as amino acids that do not occur in nature, as exemplified by 4-hydroxyproline, ?-carboxyglutamate, O-phosphoserine, N-acetylserine, N-formylmethionine, 3-methylhistidine, 5-hydroxylysine and other such amino acids and imino acids.

The term “peptidomimetic” or “peptide mimetic” describes a peptide analog, such as those commonly used in the pharmaceutical industry as non-peptide drugs, with properties analogous to those of the template peptide. (Fauchere, J., Adv. Drug Res., 15: 29 (1986) and Evans et al., J. Med. Chem., 30:1229 (1987)). Generally, peptidomimetics are structurally similar to a paradigm polypeptide (i.e., a polypeptide that has a biochemical property or pharmacological activity), but have one or more peptide linkages optionally replaced by a linkage such as, —CH₂NH—, —CH₂S—, —CH₂—CH₂—, —CH═CH—(cis and trans), —COCH₂—, —CH(OH)CH₂—, and —CH₂SO—, by methods known in the art. Advantages of peptide mimetics over natural polypeptide embodiments may include more economical production, greater chemical stability, altered specificity, reduced antigenicity, and enhanced pharmacological properties such as half-life, absorption, potency and efficacy.

As used herein, the terms “pharmaceutically acceptable,” “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The terms “protein” and “polypeptide” are used to describe a native protein, a peptide, a protein fragment, or an analog of a protein or polypeptide. These terms may be used interchangeably.

As used herein the term “reactive oxygen species” means antibody-generated oxygen species. In some embodiments, the reactive oxygen species are “neutrophil-generated,” for example, because neutrophils have antibodies on their surface. These reactive oxygen species can possess one or more unpaired electrons, or are otherwise reactive because they are readily react with other molecules. Such reactive oxygen species include but are not limited to superoxide free radicals, hydrogen peroxide, hydroxyl radicals, peroxyl radicals, ozone and other short-lived trioxygen adducts that have the same chemical signature as ozone.

Catalytic Activity of Antibodies

According to the invention, all antibodies have a previously unrecognized chemical potential that is intrinsic to the antibody molecule itself. All antibodies-studied, regardless of source or of antigenic specificity, can convert singlet oxygen into reactive oxygen species such as to ozone (O₃), superoxide radical (O₂?), hydrogen peroxide (H₂O₂), peroxyl radical or hydroxyl radical (OH^{?}). The antibody is therefore more properly perceived to be a remarkable adaptor molecule, having evolved both targeting and catalytic functions that place it at the frontline of the vertebrate defense against foreign invaders.

The ability to produce reactive oxygen species from singlet oxygen is present in intact immunoglobulins and well as in antibody fragments such as Fab, F(ab′)₂ and Fv fragments (see examples). This activity does not reside in other molecules, including RNaseA, superoxide dismutase, and Bowman-Birk inhibitor protein that can be oxidized (example I and Table 1). Also, the activity is not associated with the presence of disulfides in a molecule, even though such disulfides are sufficiently electron rich that they can be oxidized (Bent et al., J. Am. Chem. Soc., 87:2612-2619 (1975)).

The ability of an antibody to generate a reactive oxygen species from singlet oxygen is abolished if the antibody is denatured. This indicates that the three dimensional structure of antibodies is relevant to the reduction process used to generate superoxide.

The ability to produce reactive oxygen species in an efficient and long term manner from singlet oxygen is present in immunoglobulins and in the T-cell receptor (example II, FIG. 1F). The T-cell receptor shares a similar arrangement of its immunoglobulin fold domains with antibodies (Garcia et al., Science, 274:209 (1996)). However, possession of this structural motif does not appear necessary to confer a hydrogen peroxide-generating ability on proteins. β₂-macroglobulin, a member of the immunoglobulin superfamily having this structural motif, does not generate hydrogen peroxide (Welinder et al., Mol. Immunol., 28:177 (1991)).

Structural studies also indicate that a conserved tryptophan residue found in T-cell receptors resides in a domain similar to that found in antibodies. The sequence and structure surrounding the conserved tryptophan residue is highly conserved between antibodies and T-cell receptors, indicating that those surrounding structures may also play a role in allowing catalysis of singlet oxygen to reactive oxygen species.

Moreover, according to the invention, neutrophils can generate reactive oxygen species when they are activated. The catalytic activities of antibodies and neutrophils can be used to detect immunological reactions, inflammation and neutrophil activation.

Methods for Detecting Immunological and Inflammatory Responses

The invention provides methods for detecting humoral and cellular-based immune and inflammatory responses. The methods utilize the newly discovered abilities of antibodies and neutrophils to reduce singlet oxygen to reactive oxygen species.

In one embodiment, the invention provides a method for assaying for an immunological response or for an inflammatory response in a mammal comprising: (a) administering a chemical probe for a reactive oxygen species; (b) obtaining a sample from the mammal; and (c) analyzing the sample for oxidation products of the chemical probe.

In another embodiment, the invention provides an in vitro assay for neutrophil activity comprising: (a) obtaining a neutrophil sample from a mammal; (b) activating neutrophils in the neutrophil sample; and (c) observing whether a reactive oxygen species can be detected in the neutrophil sample.

In yet another embodiment, the invention provides a method for identifying an agent that can modulate neutrophil activity comprising: (a) obtaining a neutrophil sample from a mammal; (b) exposing the neutrophil sample to a test agent; (c) activating neutrophils in the neutrophil sample; and (d) quantifying the amount of reactive oxygen species generated by the neutrophil sample.

These assays are simple to perform because the basic requirements for these assays include a chemical probe for reactive oxygen species and the subject or sample to be tested. The production of reactive oxygen species can, in some instances, be enhanced through the use of a source of singlet oxygen that acts as a substrate for antibody-mediated production of reactive oxygen species. However, singlet oxygen is produced in vivo so administration of a source of singlet oxygen may not be needed.

Molecules that can provide a source of singlet oxygen include molecules that generate singlet oxygen without the need for other factors or inducers and “sensitizer” molecules that can generate singlet oxygen after exposure to an inducer. Examples of molecules that can generate singlet oxygen without the need for other factors or inducers include, but are not limited to, endoperoxides. In some embodiments, the endoperoxide employed can be an anthracene-9,10-dipropionic acid endoperoxide. Examples of sensitizer molecules include, but are not limited to, pterins, flavins, hematoporphyrins, tetrakis(4-sulfonatophenyl)porphyrin, bipyridyl ruthenium(II) complexes, rose Bengal dyes, quinones, rhodamine dyes, phthalocyanines, hypocrellins, rubrocyanins, pinacyanols or allocyanines.

Sensitizer molecules can be induced to generate singlet oxygen when exposed to an inducer. One such inducer is light. Such light can be visible light, ultraviolet light, or infrared light, depending upon the type and structure of the sensitizer.

Reactive oxygen species that can be detected by the methods of the invention include any antibody-generated oxygen species and any neutrophil-generated oxygen species. Examples of such reactive oxygen species include, but are not limited to, superoxide radical (O₂?), hydroxyl radical (OH^{?}), peroxyl radical, hydrogen peroxide (H₂O₂) or ozone (O₃). The presence of such powerful reactive oxygen species is indicative of an increased humoral immune response (e.g. increased circulating antibodies) or an increased cellular or tissue related inflammatory response (e.g. neutrophil activation). The types of immunological and inflammatory responses that can be detected are discussed in more detail below.

The invention therefore provides methods for detecting antibodies. All antibody molecules belong to a family of plasma proteins called immunoglobulins. Their basic building block, the immunoglobulin fold or domain, is used in various forms in many molecules of the immune system and other biological recognition systems. A typical immunoglobulin has four polypeptide chains, contains an antigen-binding region known as a variable region, and contains a non-varying region known as the constant region.

Any antibody can be detected using the methods of the invention. Moreover, the antibody can be in any of a variety of forms so long as it can catalyze the production of reactive oxygen species, including a whole immunoglobulins, Fv, Fab, F(ab′)₂, or other fragments, and single chain antibodies that include the variable domain complementarity determining regions (CDR), or other forms. All of these terms fall under the broad term “antibody” as used herein. The present invention contemplates detection of any type of antibody and is not limited to antibodies that recognize and immunoreact with a specific antigen. However, for some applications, the antibody or fragment thereof is immunospecific for an antigen.

The term “antibody” as used in this invention includes intact molecules as well as fragments thereof, such as Fab, F(ab′)₂, and Fv, which are capable of binding an epitope. These antibody fragments retain some ability to selectively bind with its antigen or receptor and are defined as follows:

(1) Fab, the fragment, which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) F(ab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“sFv”), defined as a genetically engineered molecule containing the variable region of the light chain, the variable region of the heavy chain, linked by a suitable polypeptide linker as a genetically fused single chain molecule.

The antibody can be detected in any mammalian or bird species or in any sample from a mammalian or bird species. Such mammals and birds include humans, dogs, cats, and livestock, for example, horses, cattle, sheep, goats, chickens, turkeys and the like. Samples from such mammals and birds can be obtained for testing. Such samples can, for example, be tissue samples or bodily fluids such as whole blood, serum, plasma, synovial fluid, lymph, urine, saliva, mucus or tears.

Chemical Probes for Reactive Oxygen Species

The reactive oxygen species produced by antibodies and neutrophils can be detected with chemical probes. Chemical probes for reactive oxygen species include any natural or synthetic compound that contains an alkene that can be oxidized and that generates a detectable oxidation product. Examples of chemical probes for reactive oxygen species include 3-vinyl-benzoic acid, 4-vinyl-benzoic acid, indigo carmine, stilbene, cholesterol and the like. Upon oxidation such chemical probes generate oxidation products such as ketones, aldehydes, ethers and related products.

For example, the structures of 3-vinyl-benzoic acid (3) and 4-vinyl-benzoic acid (4) chemical probes and the oxidation products (5a, 5b, 6a and 6b) generated by reaction of these chemical probes with reactive oxygen species are depicted below:

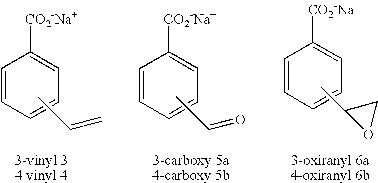

Another example of a useful chemical probe for reactive oxygen species is indigo carmine (1), which is converted into a cyclic α-ketoamide (isatin sulfonic acid, 2) by reactive oxygen species. These compounds are shown below.

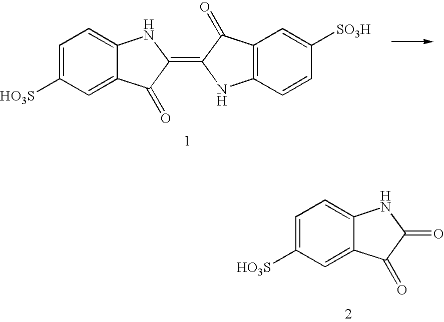

In some embodiments, one of skill in the art may choose to detect particular reactive oxygen species, for example, ozone. Ozone can be detected and distinguished from other reactive oxygen species, for example, by using indigo carmine. Cleavage of indigo carmine by ozone (O₃) can be distinguished from cleavage of indigo carmine by ¹O₂* by using isotopes. For example, ¹⁸O is incorporated into the lactam carbonyl groups of cyclic α-ketoamide 2 when ozone was the oxidant. No such ¹⁸O incorporation into the lactam carbonyl group of cyclic α-ketoamide 2 occurred when ¹O₂* was the oxidant.

The oxidation products of the chemical probe can be detected by high pressure liquid chromatography, mass spectrometry, ultraviolet light spectrophotometry, visible light spectrophotometry, liquid chromatography, gas spectrometry, liquid chromatography linked mass spectrometry, using a fluorescent means, such as with fluorescent microscopy or fluorescent spectrometry. Exemplary assay methods are performed as described in the Examples.

Thus, in some embodiments a chemical probe is administered to a mammal and a sample of the mammal's bodily fluids is collected to ascertain whether oxidation products of the chemical probe have been generated. If such oxidation products have been generated, the mammal may have an inflammation, or a heightened immune response. In other embodiments, the chemical probe is added to an in vitro assay of a bodily fluid from a mammal and the assay mixture is tested to see whether oxidation products of the chemical probe are present. Such an in vitro assay is useful, for example, to ascertain whether the bodily fluid has heightened levels of activated neutrophils.

Endogenous Production of Singlet Oxygen

The role of the newly discovered chemical potential of antibodies in vivo is dependent on the availability of the key substrate ¹O₂*. However, ¹O₂* is produced during a variety of physiological events and is available in vivo. See J. F. Kanofsky Chem.-Biol. Interactions 70, 1 (1989) and references therein. For example, ¹O₂* is produced including reperfusion. X. Zhai and M. Ashraf Am. J. Physiol.269 (Heart Circ. Physiol. 38) H1229 (1995). Also, ¹O₂* is produced in neutrophil activation during phagocytosis. J. R. Kanofsky, H. Hoogland, R. Wever, S. J. Weiss J. Biol. Chem. 263, 9692 (1988); Babior et al., Amer. J. Med., 109:33-34 (2000). Singlet oxygen (¹O₂) also results from irradiation by light of metal-free porphyrin precursors that are present in the skin of porphyria sufferers.

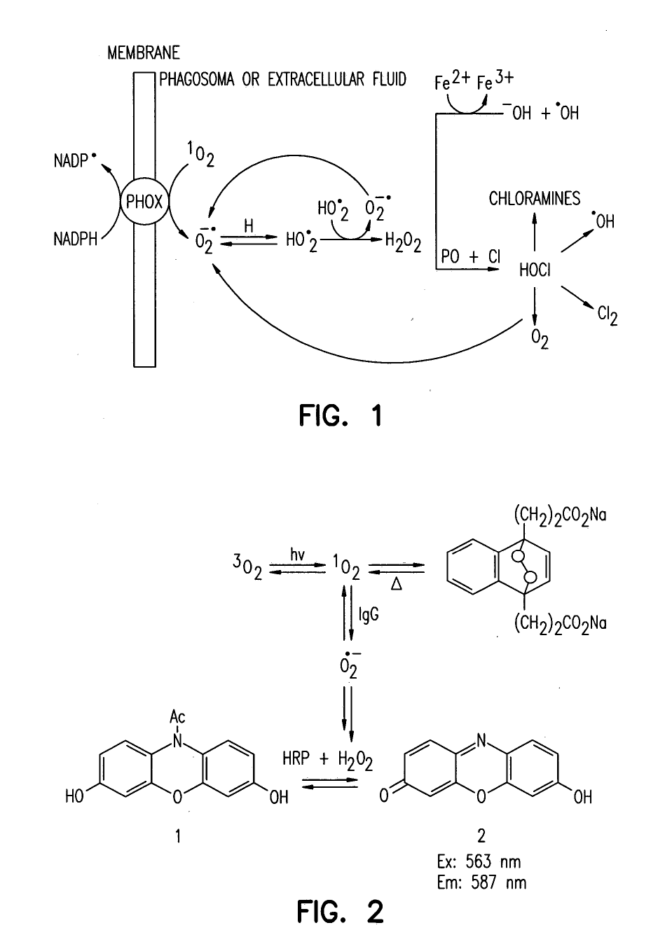

Moreover, the substrate ¹O₂* is generated by phagocytosis or reperfusion in amounts that are sufficient for antibodies to produce detectable levels of reactive oxygen species. For example, the volume of the phagosome is approximately 1.0×10⁻¹⁵ liters. Hence, the reactions identified herein need not be highly efficient because only a few hundred molecules comprise micromolar concentrations in such a small volume. In fact, the concentration of ¹O₂* has been calculated to be as high as a molar concentration within the phagosome. E. P. Reeves et al., Nature 416, 291 (2002). The same estimates can be made regarding the number of antibody molecules from titrations with bacteria and fluorescently-labeled antibodies and the immuno-gold studies (FIG. 2). These analyses suggest that there are about 10⁵ antibody molecules bound to each bacterium and such amounts would correspond to a millimolar antibody concentration within the phagosome. Thus, by even the most conservative of estimates, the concentrations of ¹O₂* and antibody within the phagosome far exceed those used in the illustrative examples provided here.

Singlet molecular oxygen (¹O₂) is also generated during microbicidal processes in both direct and indirect ways. Singlet molecular oxygen (¹O₂) is generated directly, for example, via the action of flavoprotein oxidases (Allen, R. C., Stjernholm, R. L., Benerito, R. R. & Steele, R. H., eds. Cormier, M. J., Hercules, D. M. & Lee, J. (Plenum, New York), pp. 498-499 (1973); Klebanoff, S. J. in The Phagocytic Cell in Host Resistance (National Institute of Child Health and Human Development, Orlando, Fla.) (1974)). Alternatively, ¹O₂ can be generated indirectly microbicidal processes such as the nonenzymatic disproportionation of O₂^{?}? in solutions at low pH, like those found in the phagosome (Reaction 3) (Stauff, J., Sander, U. & Jaeschke, W., Chemiluminescence and Bioluminescence, eds., Williams, R. C. & Fudenberg, H. H. (Intercontinental Medical Book Corp., New York), pp. 131-141 (1973); Allen, R. C., Yevich, S. J., Orth, R. W. & Steele, R. H., Biochem. Biophys. Res. Commun., 60, 909-917 (1974)).

Because ¹O₂ is so highly reactive, it was previously considered to be an endpoint in the cascade of oxygen-scavenging agents. However, it has been found that antibodies and neutrophils can intercept ¹O₂ and efficiently reduce it to reactive oxygen species, thereby providing a means for in vivo detection of immunological responses, inflammation and neutrophil activation.

Immunological and Inflammation Responses

Causes of immunological and inflammatory responses are generally categorized as either infectious or non-infectious. The main infection and disease-fighting cell of the human immune system is the white blood cell (leukocyte), which circulates through the blood. Leukocytes are produced by the bone marrow, which generates neutrophils, platelets, erythrocytes, lymphocytes, and other leukocytes. Approximately 50 to 65 percent of all leukocytes are “neutrophils.” When the hematopoietic system is functioning correctly, platelets and neutrophils proliferate rapidly and turn over at a high rate, unlike the lymphocytes and red blood cells, which are long-lived.

During an immune response, activation and differentiation of B lymphocytes leads to the secretion of high affinity antigen-specific antibodies that can be detected by the methods of the invention. Antibody production is often associated with infection. According to the invention any type of infection can be detected. Infectious diseases involving bacteria and viruses and other parasites can be detected by the methods of the invention. Examples of infective entities that can be detected include microbes, viruses, parasites and the like. Microbes that may be detected include, but are not limited to microbes such as Staphylococcus aureus, Salmonella typhi, Escherichia coli, Escherichia coli O157:H7, Shigella dysenteria, Psuedomonas aerugenosa, Pseudomonas cepacia, Vivrio cholerae, Helicobacter pylori, a multiply-resistant strain of Staphylococcus aureus, a vancomycin-resistant strain of Enterococcus faecium, or a vancomycin-resistant strain of Enterococcus faecalis.

Viral infections that can be detected include, but are not limited to viral infections such as hepatitis A virus, hepatitis B virus, hepatitis C virus, human immunodeficiency virus, poxvirus, herpes virus, adenovirus, papovavirus, parvovirus, reovirus, orbivirus, picomavirus, rotavirus, alphavirus, rubivirus, influenza virus type A, influenza virus type B, flavivirus, coronavirus, paramyxovirus, morbillivirus, pneumovirus, rhabdovirus, lyssavirus, orthmyxovirus, bunyavirus, phlebovirus, nairovirus, hepadnavirus, arenavirus, retrovirus, enterovirus, rhinovirus or filovirus.

Inflammation is the reaction of vascularized tissue to local injury. This injury can have a variety of causes, including infections and direct physical injury. Upon injury, the clotting system and plasmin systems are initiated together with the appropriate nervous system response to generate an initial response to facilitate immune activation. Increased blood flow, capillary permeability and chemotactic factors, including those of the complement cascade, modulate neutrophil migration to the damaged site. Neutrophils are the predominant cell type involved in acute inflammation, whereas lymphocytes and macrophages are more prevalent in chronic inflammation.

The inflammatory response can be considered beneficial, because without it, infections would go unchecked, wounds would never heal, and tissues and organs could be permanently damaged and death may ensue.

However, inflammation can also be potentially harmful. During inflammation activated neutrophils release a variety of degradative enzymes, including proteolytic and oxidative enzymes into the surrounding extracellular environment. The substances released by neutrophils can cause potentially harmful side effects. Though the half-life of circulating neutrophils is 6-8 hours, the extravascular survival of the activated cells can approach four days. The numbers of activated neutrophils and their degree of activation is directly related to tissue injury. In vivo, as neutrophils die, they are recognized and phagocytosed by tissue macrophages, a process which is critical for resolution of the inflammatory response. In vitro, neutrophils undergo spontaneous apoptosis over a period of several days, which can be either enhanced or inhibited by cytokines and other mediators. Phagocytosis of dying neutrophils is now recognized as the prime mode of resolving inflammation (J. Savill, J. Leukoc. Biol., 61:375, 1997).

Non-infectious diseases in which neutrophils play a role in tissue damage include gout, rheumatoid arthritis, arthritis, immune vasculitis, neutrophil dermatoses, glomerulonephritis, inflammatory bowel disease, myocardial infarction, ARDS (adult respiratory distress syndrome), asthma, emphysema and malignant neoplasms. Inflammation causes the pathologies associated with myocardial infarction, ischemic reperfusion injury, hypersensitivity reactions, renal diseases, aberrant smooth muscle disorder, liver diseases, proliferation of cancer cells, inflammation in cancer patients receiving radiotherapy, vasculitis, glomerulonephritis, systemic lupus erythematosus, adult respiratory distress syndrome, ischemic diseases, heart disease, stroke, intestinal ischemia, reperfusion injury, hemochromatosis, acquired immunodeficiency syndrome, emphysema, organ transplantation, gastric ulcers, hypertension, preeclampsia, neurological diseases (multiple sclerosis, Alzheimer's disease, Parkinson's disease, amyotrophic lateral sclerosis, and muscular dystrophy) alcoholism and smoking-related diseases.

Millions of people each year are treated for the above conditions in the U.S. However, before an appropriate treatment can be devised, inflammation must be detected and categorized as either infectious or non-infectious.

Screening for Modulators of the Immune Response

The invention also provides methods for identifying agents that can modulate neutrophil activity. Such methods can include the steps of (a) obtaining a neutrophil sample from a mammal; (b) exposing the neutrophil sample to a test agent; (c) activating neutrophils in the neutrophil sample; and (d) quantifying an amount of reactive oxygen species generated by the neutrophil sample.

Other embodiments include comparing the signal generated by the neutrophil sample with a suitable control. Such a suitable control can be a control sample of the same type of neutrophil sample that has not been exposed to the test agent. Use of this type of control can facilitate analysis of whether the test agent has any affect on neutrophil activation.

In other embodiments, the method can also include contacting the neutrophil sample with a reagent that can generate singlet oxygen from molecular oxygen. Such methods can also include irradiating the mixture of the sample, the chemical probe and the reagent that generate singlet oxygen. The antibodies on the neutrophils reduce singlet oxygen to superoxide or hydrogen peroxide or ozone by the antibody.

The irradiating step is performed with infrared light, ultraviolet light or visible light, the selection of which is dependent on the sensitizer.

The formed reactive oxygen species is detected by procedures described herein.

In a separate screening method of the present invention, a method for performing an immunoassay to detect antibody immunoreactivity with an antigen is also contemplated. The method comprises the steps of:

A. contacting in a singlet oxygen-generating medium a substrate having immobilized thereon a composition comprising a first reagent comprising an antigen or an antibody, with a second composition comprising an antigen or an antibody that is reactive with the first reagent to form an immobilized antigen-antibody complex, wherein the antibody generates superoxide or hydrogen peroxide from singlet oxygen in the presence of oxygen; and

B. detecting the antibody-generated reactive oxygen species, thereby detecting the antibody immunoreactivity with the antigen.

The reaction and detection means are those as described herein. In one aspect, the first composition is an antigen and the second composition is an antibody. In the opposite aspect, the first composition is an antibody and the second composition is an antigen.

The invention further contemplates a similar method for performing an immunoassay to detect antibody immunoreactivity with an antigen where an antigen is immobilized and contacted with an antibody composition.

Such immunoassay methods are an improvement over those that are well known as methods to assess antigen-antibody immunoreactivity and to identify antigens and/or antibodies. The advantage of the present method over previous other immunoassay methods lies in the present elimination of at least one method step and/or the incorporation of a secondary labeled immunoreactive molecule, the labeling either being a radioactive or enzymatic compound.

In the present invention, the minimum requirements are singlet oxygen, an antibody reagent, an antigen reagent, and a chemical probe that reacts with reactive oxygen species generated from the antibody. One such reactant that can be used is AMPLEX™ Red. It is a commercially available reagent sold by Molecular Probes (Eugene, Oreg.) for reacting antibody generated hydrogen peroxide in the immunoassay. It is sold in a kit that provides a one-step fluorometric method for measuring hydrogen peroxide using a fluorescent microplate or fluorimeter for detection. The assay is based on the detection of hydrogen peroxide using 10-acetyl-3,7-dihyroxyphenoxazine, a highly sensitive and stable probe for hydrogen peroxide. In the presence of horseradish peroxidase, the AMPLEX™ Red reagent reacts with hydrogen peroxide in a 1:1 stoichiometry to produce highly fluorescent resorufin, that provides a detection mechanism to detect as little as 10 picomoles of hydrogen peroxide in a 200 microliter volume.

In contrast, prior immunoassay techniques, including radioimmunoassays (RIA), enzyme-immunoassays (EIA), and the classic enzyme-linked immunosorbent assay (ELISA), all require either the use of a radioactively labeled immunoreactive molecule as in RIA or an additional labeled immunoreactive molecule. The present invention neither requires potentially harmful radioactive isotopes to label a molecule nor requires an additional immunoreactive reagent that generally is referred to as a secondary antibody that is usually conjugated with an enzyme to allow for the detection of the complex formed with the first antibody with the antigen. In the latter assays, the reaction of the secondary antibody with the formed antigen-antibody complex (generally through an anti-first antibody specificity immunoreactivity) is detected through a color-producing substrate solution specific for the conjugated enzyme. In summary, in the present invention, the antibody mediated generation of hydrogen peroxide is detected with high detection capacity without radioactive agents, without requiring an additional reagent and/or admixing step such as those practiced in U.S. Pat. No. 3,905,767; 4,016,043; U.S. RE 032696; and U.S. Pat. No. 4,376,110, the disclosures of which are hereby incorporated by reference.

Therapeutic Methods

The invention provides methods for the production of oxidants when their production is warranted, such as for inhibiting microbial infection, in promoting wound healing, lysing bacteria, eliminating viruses, targeting cancer cells for oxidant-induced lysis and the like processes. For example, the invention provides antibody mediated generation of reactive oxygen species to combat a bacterial infection or viral infection. The reactive oxygen species acts as an anti-microbial agent destroying the bacteria or the viruses. Thus, to enhance this process, one would use the method of this invention to provide an antibody composition to the area to cause an increase in the local concentration of reactive oxygen species.

Therapeutic methods contemplated by the invention are based on using an antibody that can generate reactive oxygen species from singlet oxygen include 1) inhibiting proliferation of a microbe, or targeting and killing a microbe in a patient where the antibody recognizes and immunoreacts with an antigen expressed on the microbe, 2) inhibiting proliferation of a cancer cell or targeting and killing a cancer cell in a patient where the antibody recognizes and immunoreacts with an antigen expressed on the cancer cell, 3) inhibiting tissue injury associated with neutrophil mediated inflammation in a subject, for example where the inflammation results from a bacterial infection or when the subject has an autoimmune disease, 4) enhancing the bactericidal effectiveness of a phagocyte in a subject, 5) promoting wound healing in a subject having a open wound where the ozone, superoxide or hydrogen peroxide stimulates fibroblast proliferation and/or the immune response further includes lymphocyte proliferation, 6) stimulating cell proliferation, such as stimulating fibroblast proliferation in a wound in a subject, and similar situations.

In some embodiments, the invention provides therapeutic methods for treating microbial infections and other diseases that benefit from enhanced production of a reactive oxygen species such as a superoxide radical, hydroxyl radical, ozone or hydrogen peroxide. Such methods can employ any antibody to generate a reactive oxygen species in a situation where the production of such a reactive oxygen species is warranted.

The present invention also contemplates the use of engineered molecules including engineered antibodies that have been altered to contain an additional reductive center, the presence of which provides added capability to generate a reactive oxygen species from singlet oxygen when such production is desired. The use of engineered molecules having more than two reductive centers compared to a non-engineered antibody having the two conserved tryptophan residues is warranted when enhanced production of a reactive oxygen species is needed.

In still further aspects, the antibody is a recombinant antibody that is provided as above or, alternatively, is expressed from an expression vector delivered to the cell. The expression vector in this context can also express a sensitizer molecule (see below).

In one embodiment, the invention contemplates a method for inhibiting the growth of a microbe where the microbe is contacted with a composition including an antibody able to generate such a reactive oxygen species from singlet oxygen. The method is successful with either nonspecific or immunospecific (antigen binding) whole or fragment antibodies. Such antibody fragments include single chain antibodies as well as the engineered molecules and antibodies described herein. However, when localized activity against a microbe is desired, the antibody can be specific for an antigen associated with the microbe. For example, the antibody can bind selectively to an antigen on the surface of the microbe.

The antibody composition can be delivered in vivo to a subject with a microbial infection or other disease or condition that may benefit from exposure to a reactive oxygen species. Preferred in vivo delivery methods include administration intravenously, topically, by inhalation, by cannulation, intracavitally, intramuscularly, transdermally, subcutaneously or by liposome containing the antibody.

Exemplary concentrations of antibody at the cell surface range from 1 to 5 micromolar. However, the concentration may vary depending on the desired outcome where the amount of antibody provided is that amount of antibody that is sufficient to obtain the desired physiological effect, i.e., the generation of a reactive oxygen species or a derivative oxidant thereof to generate oxidative stress. Dosing and timing of the therapeutic treatments with antibody compositions are compatible with those described for antioxidants below.

The methods of the invention further contemplate exposing an antibody-antigen complex to irradiation with ultraviolet, infrared or visible light in the method of generating antibody-mediated reactive oxygen species or derivative oxidants thereof. To enhance the production of a reactive oxygen species, a reactive oxygen species-generating amount of a photosensitizer, also referred to as a sensitizer, can be utilized in the therapeutic methods described herein. As defined herein, a sensitizer is any molecule that induces or increases the concentration of singlet oxygen. Sensitizers can be used in the presence of irradiation, the process of which includes exposure to ultraviolet, infrared or visible light for a period sufficient to activate the sensitizer. Exemplary exposure times and conditions are described in the examples.

A reactive oxygen species-generating amount of sensitizer is the amount of sensitizer that is sufficient to obtain the desired physiological effect, e.g., generation of a reactive oxygen from singlet oxygen, mediated by an antibody in any situation where the presence of such reactive oxygen species and the derivatives thereof is warranted. In some embodiments, a sensitizer is conjugated to the antibody. An antibody conjugated to a sensitizer is generally capable of binding to a antigen, i.e., the antibody retains an active antigen binding site, allowing for antigen recognition and complexing to occur.

Exemplary sensitizers include but are not limited to pterins, flavins, hematoporphyrin, tetrakis(4-sulfonatophenyl)porphyrin, bipyridyl ruthemium(II) complexes, rose bengal dye, quinones, rhodamine dyes, phtalocyanine, and hypocrellins.

In a further embodiment, generation of a reactive oxygen species is enhanced by administering a means to enhance production of singlet oxygen. Reduced singlet oxygen is the source of reactive oxygen species or derivative oxidants thereof. One means to enhance production of singlet oxygen is a prodrug that includes any molecule, compound, or reagent that is useful in generating singlet oxygen. Such a prodrug is administered with, or at a time subsequent to, the administering or contacting of an antibody with a desired target cell, tissue or organ as described herein. When a prodrug is administered after antibody administration, the antibody has already had an opportunity to immunoreact with its target antigen and form an antibody-antigen complex. The means to enhance the production of singlet oxygen can then enhance the generation of reactive oxygen species such as hydrogen peroxide, ozone, superoxide radicals or derivative oxidants thereof, at the site of antibody-antigen recognition. This embodiment has particular advantages, for example, the ability to create increased local accumulation of therapeutically desirable superoxide, ozone or hydrogen peroxide at a desired site or location.

A preferred prodrug is endoperoxide, for example, at a concentration of about 1 micromolar to about 50 micromolar. A preferred concentration of endoperoxide to achieve at the antibody-antigen complex site is about 10 micromolar.

An antigenic target of the antibodies of the invention can be any antigen known or available to one of skill in the art. The antigen can be any antigen that is present on or in a cell, tissue or organ where the presence of reactive oxygen species and the antibody mediated process of producing it is warranted. The antigen can be in solution, for example, in extracellular fluids. An antigen can be, for example, a protein, a peptide, a fatty acid, a low density lipoprotein, an antigen associated with inflammation, a cancer cell antigen, a bacterial antigen, a viral antigen or a similar molecule.

Cells on which antigens are associated include but are not limited to microbial, endothelial, interstitial, epithelial, muscle, phagocytic, blood, dendritic, connective tissue and nervous system cells.

Hence, for example, infections of the following target microbial organisms can be treated by the antibodies of the invention: Aeromonas spp., Bacillus spp., Bacteroides spp., Campylobacter spp., Clostridium spp., Enterobacter spp., Enterococcus spp., Escherichia spp., Gastrospirillum sp., Helicobacter spp., Klebsiella spp., Salmonella spp., Shigella spp., Staphylococcus spp., Pseudomonas spp., Vibrio spp., Yersinia spp., and the like. Infections that can be treated by the antibodies of the invention include those associated with staph infections (Staphylococcus aureus), typhus (Salmonella typhi), food poisoning (Escherichia coli, such as O157:H7), bascillary dysentery (Shigella dysenteria), pneumonia (Psuedomonas aerugenosa and/or Pseudomonas cepacia), cholera (Vivrio cholerae), ulcers (Helicobacter pylori) and others. E. coli serotype 0157:H7 has been implicated in the pathogenesis of diarrhea, hemorrhagic colitis, hemolytic uremic syndrome (HUS) and thrombotic thrombocytopenic purpura (TTP). The antibodies of the invention are also active against drug-resistant and multiply-drug resistant strains of bacteria, for example, multiply-resistant strains of Staphylococcus aureus and vancomycin-resistant strains of Enterococcus faecium and Enterococcus faecalis.

The anti-microbial compositions of the invention are also effective against viruses. The term “virus” refers to DNA and RNA viruses, viroids, and prions. Viruses include both enveloped and non-enveloped viruses, for example, hepatitis A virus, hepatitis B virus, hepatitis C virus, human immunodeficiency virus (HIV), poxviruses, herpes viruses, adenoviruses, papovaviruses, parvoviruses, reoviruses, orbiviruses, picornaviruses, rotaviruses, alphaviruses, rubivirues, influenza virus type A and B, flaviviruses, coronaviruses, paramyxoviruses, morbilliviruses, pneumoviruses, rhabdoviruses, lyssaviruses, orthmyxoviruses, bunyaviruses, phleboviruses, nairoviruses, hepadnaviruses, arenaviruses, retroviruses, enteroviruses, rhinoviruses and the filovirus.

Other therapeutic conditions that would benefit from antibody mediated reactive oxygen production in a cell, tissue, or organs as well as extracellular compartments are well known to those of ordinary skill in the art and have been reviewed by McCord, Am. J. Med., 108:652-659 (2000) and Babior et al., Am. J. Med., 109:33-44 (2000), the disclosures of which are hereby incorporated by reference.

Anti-microbial activity can be evaluated against these varieties of microbes using methods available to one of skill in the art. Anti-microbial activity, for example, is determined by identifying the minimum inhibitory concentration (MIC) of an antibody of the present invention that prevents growth of a particular microbial species. In one embodiment, anti-microbial activity is the amount of antibody that kills 50% of the microbes when measured using standard dose or dose response methods.

Methods of evaluating therapeutically effective dosages for treating a microbial infection with antibodies described herein include determining the minimum inhibitory concentration of an antibody preparation at which substantially no microbes grow in vitro. Such a method permits calculation of the approximate amount of antibody needed per volume to inhibit microbial growth or to kill 50% of the microbes. Such amounts can be determined, for example, by standard microdilution methods. For example, a series of microbial culture tubes containing the same volume of medium and the substantially the same amount of microbes are prepared, and an aliquot of antibody is added. The aliquot contains differing amounts of antibody in the same volume of solution. The microbes are cultured for a period of time corresponding to one to ten generations and the number of microbes in the culture medium is determined.

The optical density of the cultural medium can also be used to estimate whether microbial growth has occurred—if no significant increase in optical density has occurred, then no significant microbial growth has occurred. However, if the optical density increases, then microbial growth has occurred. To determine how many microbial cells remain alive after exposure to the antibody, a small aliquot of the culture medium can be removed at the time when the antibody is added (time zero) and then at regular intervals thereafter. The aliquot of culture medium is spread onto a microbial culture plate, the plate is incubated under conditions conducive to microbial growth and, when colonies appear, the number of those colonies is counted.

Compositions

The antibodies, sensitizers or chemical probes of the invention may be formulated into a variety of acceptable compositions. Such pharmaceutical compositions can be administered to a mammalian host, such as a human patient, in a variety of forms adapted to the chosen route of administration, i.e., orally or parenterally, by intravenous, intramuscular, topical or subcutaneous routes.

In cases where antibodies, sensitizers and chemical probes are sufficiently basic or acidic to form stable nontoxic acid or base salts, administration of such antibodies, sensitizers and chemical probes as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, a-ketoglutarate, and a-glycerophosphate. Suitable inorganic salts may also be formed, including hydrochloride, sulfate, nitrate, bicarbonate, and carbonate salts.

Pharmaceutically acceptable salts are obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids also are made.

Thus, the present antibodies, sensitizers and chemical probes may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the antibodies, sensitizers and chemical probes may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of oxidants and oxygen scavengers in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

For wound healing, topical application to a wound on a subject can be employed. A composition containing an antibody can be applied directly to the wound or applied to a bandage and then applied to the wound. Other therapeutic conditions that would benefit from the creation or enhancement of superoxide, ozone or hydrogen peroxide in a cell, tissue, organ or extracellular compartment are available to those of ordinary skill in the art and have been reviewed by McCord, Am. J. Med., 108:652-659 (2000), the disclosure of which are hereby incorporated by reference.

The antibodies, sensitizers and chemical probes may also be administered intravenously or intraperitoneally by infusion or injection. Solutions of the antibodies, sensitizers and chemical probes may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations may contain a preservative to prevent the growth of microorganisms.

The pharmaceutical dosage forms suitable for injection or infusion can include sterile aqueous solutions or dispersions or sterile powders comprising the antibodies, sensitizers and chemical probes that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the antibodies, sensitizers or chemical probes in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the oxidants and oxygen scavengers plus any additional desired ingredient present in the previously sterile-filtered solutions.

For topical administration, the antibodies, sensitizers or chemical probes may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions that can be used to deliver the antibodies, sensitizers or chemical probes of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the antibodies, sensitizers or chemical probes of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the antibodies, sensitizers or chemical probes of the present invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the antibodies, sensitizers or chemical probes, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The antibodies, sensitizers or chemical probes are conveniently administered in unit dosage form; for example, containing 5 to 1000 mg, conveniently 10 to 750 mg, most conveniently, 50 to 500 mg of active ingredient per unit dosage form.

Ideally, the antibodies, sensitizers or chemical probes should be administered to achieve peak plasma concentrations of the antibodies, sensitizers or chemical probes of from about 0.005 to about 75 μM, preferably, about 0.01 to 50 μM, most preferably, about 0.1 to about 30 μM. This may be achieved, for example, by the intravenous injection of a 0.05 to 5% solution of the antibodies, sensitizers or chemical probes, optionally in saline, or orally administered as a bolus containing about 1-100 mg of the antibodies, sensitizers or chemical probes. Desirable blood levels may be maintained by continuous infusion to provide about 0.01-5.0 mg/kg/hr or by intermittent infusions containing about 0.4-15 mg/kg of the antibodies, sensitizers or chemical probes.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The therapeutic compositions of this invention, antibodies that include both engineered antibodies and other molecules containing additional reductive centers as described herein for promoting antibody activity, are administered in a manner compatible with the dosage formulation, and in a therapeutically effective amount. The quantity to be administered and timing depends on the subject to be treated, capacity of the subject's system to utilize the active ingredient, and degree of therapeutic effect desired. Precise amounts of active ingredient required to be administered depend on the judgement of the practitioner and are peculiar to each individual. However, suitable dosage ranges for various types of applications depend on the route of administration. Suitable regimes for administration are also variable, but are typified by an initial administration followed by repeated doses at intervals to result in the desired outcome of the therapeutic treatment.

Therapeutic compositions of the present invention contain a pharmaceutically acceptable carrier together with the antibodies, sensitizers or chemical probes. In a preferred embodiment, the therapeutic composition is not immunogenic when administered to a mammal or human patient for therapeutic purposes.

The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions, in liquid prior to use can also be prepared. The preparation can also be emulsified.

The active ingredient can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic compositions of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Pharmaceutically acceptable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

The invention is further described in detail by reference to the non-limiting examples that follow. While the invention has been described in detail with reference to certain preferred embodiments thereof, it will be understood that modifications and variations are within the spirit and scope of that which is described and claimed.

### Example I

### Antibodies have the Intrinsic Capacity to Destroy Antigens

### Materials and Methods

Antibodies: The following whole antibodies were obtained from PharMingen: 49.2 (mouse IgG_{2b}?), G155-178 (mouse IgG₂ₐ ?), 107.3 (mouse IgG₁ ?), A95-1 (rat IgG_{2b}), G235-2356 (hamster IgG), R3-34 (rat IgG ?), R35-95 (rat IgG₂ₐ ?), 27-74 (mouse IgE), A110-1 (rat IgG₁ ?), 145-2C11 (hamster IgG group1 ?), M18-254 (mouse IgA ?), and MOPC-315 (mouse IgA ?). The following were obtained from Pierce: 31243 (sheep IgG), 31154 (human IgG), 31127 (horse IgG), and 31146 (human IgM).

The following F(ab′)₂ fragments were obtained from Pierce: 31129 (rabbit IgG), 31189 (rabbit IgG), 31214 (goat IgG), 31165 (goat IgG), and 31203 (mouse IgG). Protein A, protein G, trypsin-chymotrypsin inhibitor (Bowman-Birk inhibitor), β-lactoglobulin A, a-lactalbumin, myoglobin, β-galactosidase, chicken egg albumin, aprotinin, trypsinogen, lectin (peanut), lectin (Jacalin), BSA, superoxide dismutase, and catalase were obtained from Sigma. Ribonuclease I A was obtained from Amersham Pharmacia. The following immunoglobulins were obtained in-house using hybridoma technology: OB2-34C12 (mouse IgG₁ ?), SHOI-41G9 (mouse IgG₁ ?), OB3-14F1 (mouse IgG₂ₐ ?), DMP-15G12 (mouse IgG₂ₐ ?), ADI-19G1 (mouse IgG_{2b} ?), NTJ-92C12 (mouse IgG₁ ?), NBA-5G9 (mouse IgG₁ ?), SPF-12H8 (mouse IgG₂ₐ ?), TIN-6C₁₁ (mouse IgG₂ₐ ?), PRX-1B7 (mouse IgG₂a ?), HA5-19A11 (mouse IgG ?), EP2-19G2 (mouse IgG1 ?), GNC-92H2 (mouse IgG1 ?), WDI-6G6 (mouse IgG1 ?), CH2-5H7 (mouse IgG2b ?), PCP-21H3 (mouse IgG1 ?), and TM1-87D7 (mouse IgG1 ?). DRB polyclonal (human IgG) and DRB-b 12 (human IgG) were supplied by Dennis R. Burton (The Scripps Research Institute). 1D4 Fab (crystallized) was supplied by Ian A. Wilson (The Scripps Research Institute).

All assays were carried out in PBS (10 mM phosphate/160 mM sodium chloride, pH 7.4). Commercial protein solution samples were dialyzed into PBS as necessary. Amplex Red hydrogen peroxide assay kits (A-12212) were obtained from Molecular Probes.

Antibody/Protein Irradiation. Unless otherwise stated, the assay solution (100 μl, 6.7 μM protein in PBS, pH 7.4) was added to a glass vial, sealed with a screw-cap, and irradiated with either UV (312 nm, 8000 μWcm⁻² Fischer-Biotech transilluminator) or visible light.

Quantitative Assay for Hydrogen Peroxide. An aliquot (20 μl) from the protein solution was removed and added into a well of a 96-well microtiter plate (Costar) containing reaction buffer (80 μl). Working solution (100 μl/400 μM Amplex Red reagent ½ units/ml horseradish peroxidase) was then added, and the plate was incubated in the dark for 30 min. The fluorescence of the well components was then measured using a CytoFluor Multiwell Plate Reader (Series 4000, PerSeptive Biosystems, Framingham, Mass.; Ex/Em: 530/580 nm). The hydrogen peroxide concentration was determined using a standard curve. All experiments were run in duplicate, and the rate is quoted as the mean of at least two measurements.

Sensitization and Quenching Assays. A solution of 31127 (100 μl of horse IgG, 6.7 μM) in PBS (pH 7.4, 4% dimethylformamide) and hematoporphyrin IX (40 μM) was placed in proximity to a strip light. Hydrogen peroxide concentration was determined as described herein. The assay was also performed in the presence of NaN₃ (100 mM) or PBS in D₂O.

Oxygen Dependence. A solution of 31127 (1.6 ml, horse IgG, 6.7 μM) in PBS (pH 7.4) was rigorously degassed using the freeze/thaw method under argon. Aliquots (100 μl) were introduced into septum-sealed glass vials that had been purged with the appropriate O₂/Ar mixtures (0-100%) via syringe. Dissolved oxygen concentrations were measured with an Orion 862A dissolved oxygen meter. These solutions were then vortexed vigorously, allowed to stand for 20 min, and then vortexed again. A syringe containing the requisite O₂/Ar mixture was used to maintain atmospheric pressure during the course of the experiment. Aliquots (20 μl) were removed using a gas-tight syringe and hydrogen peroxide concentration measured as described herein. The data from three separate experiments were collated and analyzed using the Enzyme Kinetics v1.1 computer program (for determination of Vₘₐₓ and Kₘ parameters).

Antibody Production of Hydrogen Peroxide in the Dark, Using a Chemical ¹O₂ Source. A solution of sheep IgG 31243 (100 μl, 20 μM) in PBS (pH 7.4) and the endoperoxide of disodium 3,3N-(1,4-naphthylidene) dipropionate (25 mM in D₂O) were placed in a warm room (37EC) for 30 min in the dark. Hydrogen peroxide concentration was determined as described herein.

Hydrogen Peroxide Formation by the Fab 1D4 Crystal. A suspension of crystals of the Fab fragment of 1D4 (2 μl) was diluted with PBS (198 μl, pH 7.4) and vortexed gently. Following centrifugation, the supernatant was removed, and the washing procedure was repeated twice further. The residual crystal suspension was then diluted into PBS, pH 7.4 (100 μl), and added into a well of a quartz ELISA plate. Following UV irradiation for 30 min, Amplex Red working solution (100 μl) was added, and the mixture was viewed on a fluorescence microscope.

Antibody Fluorescence Versus Hydrogen Peroxide Formation. A solution of 31127 (1.0 ml of horse IgG, 6.7 μM) in PBS (pH 7.4) was placed in a quartz cuvette and irradiated with UV light for 40 min. At 10-min intervals, the fluorescence of the solution was measured using an SPF-500C spectrofluorimeter (SLM-Aminco, Urbana, IL; Ex/Em, 280/320). At the same time point, an aliquot (20 μl) of the solution was removed, and the hydrogen peroxide concentration was determined as described herein.

Consumption of Hydrogen Peroxide by Catalase. A solution of EP2-19G 12 (100 μl of mouse IgG, 20 μM in PBS, pH 7.4) was irradiated with UV light for 30 min, after which time the concentration of hydrogen peroxide was determined by stick test (EM Quant Peroxide Test Sticks) to be 2 mg/liter. Catalase [1 μl, Sigma, 3. 2 M (NH₄)₂SO₄, pH 6.0] was added, and after 1 min, the concentration of H₂O₂ was found to be 0 mg/liter.

Denaturation. IgG 19G12 (100 μl, 6.7 μM) was heated to 100EC in an Eppendorf tube for 2 min. The resultant solution was transferred to a glass, screw-cap vial and irradiated with UV light for 30 min. The concentration of H₂O₂ was determined after 30 min.

### Results and Discussion

The measured values for the initial rate of formation of hydrogen peroxide by a panel of intact immunoglobulins and antibody fragments are collected in Table 1. It is believed that Ig-generated O₂^{?}? dismutates spontaneously into H₂O₂, which is then utilized as a cosubstrate with N-acetyl-3,7-dihydroxyphenazine 1 (Amplex Red) for horseradish peroxidase, to produce the highly fluorescent resorufin 2 (excitation maxima 563 nm, emission maxima 587 nm) (FIG. 2) (Zhou, M., Diwu, Z., Panchuk-Voloshina, N. & Haugland, R. P., Anal. Biochem., 253, 162-168 (1997)). To confirm that irradiation of the buffer does not generate O₂^{?}? and that the antibodies are not simply acting as protein dismutases (Petyaev, I. M. & Hunt, J. V., Redox Report, 2, 365-372 (1996)), the enzyme superoxide dismutase was irradiated in PBS. Under these conditions, the rate of hydrogen peroxide generation is the same as irradiation of PBS alone.

TABLE 1
Production of hydrogen peroxide* by immunoglobulins

| Entry | Clone | Source | Isotype | Rate,^{H} nmol/min/mg |
|---|---|---|---|---|
| 1 | CH25H7 | Mouse | IgG2b, ? | 0.25 |
| 2 | WD16G6 | Mouse | IgG1, ? | 0.24 |
| 3 | SHO-141G9 | Mouse | IgG1, ? | 0.26 |
| 4 | OB234C12 | Mouse | IgG1, ? | 0.22 |
| 5 | OB314F1 | Mouse | IgG2a, ? | 0.23 |
| 6 | DMP15G12 | Mouse | IgG2a, ? | 0.18 |
| 7 | AD19G1 | Mouse | IgG2b, ? | 0.22 |
| 8 | NTJ92C12 | Mouse | IgG1, ? | 0.17 |
| 9 | NBA5G9 | Mouse | IgG1, ? | 0.17 |
| 10 | SPF12H8 | Mouse | IgG2a, ? | 0.29 |
| 11 | TIN6C11 | Mouse | IgG2a, ? | 0.24 |
| 12 | PRX1B7 | Mouse | IgG2a, ? | 0.22 |
| 13 | HA519A4 | Mouse | IgG1, ? | 0.20 |
| 14 | 92H2 | Mouse | IgG1, ? | 0.41 |
| 15 | 19G2 | Mouse | IgG1, ? | 0.20 |
| 16 | PCP-21H3 | Mouse | IgG1, ? | 0.97 |
| 17 | TM1-87D7 | Mouse | IgG1, ? | 0.28 |
| 18 | 49.2 | Mouse | IgG2b, ? | 0.24 |
| 19 | 27-74 | Mouse | IgE, std. | 0.36 |
| | | | isotype | |
| 20 | M18-254 | Mouse | IgA, ? | 0.39 |
| 21 | MOPC-315 | Mouse | IgA, ? | 0.39 |
| 22 | 31203 | Mouse | F(ab')₂ | 0.21 |
| 23 | b12 | Human | IgG | 0.45 |
| 24 | polyclonal | Human | IgG | 0.34 |
| 25 | 31154 | Human | IgG | 0.18 |
| 26 | 31146 | Human | IgM | 0.22 |
| 27 | R3-34 | Rat | IgG1, ? | 0.27 |
| 28 | R35-95 | Rat | IgG2a, ? | 0.17 |
| 29 | A95-1 | Rat | IgG2b | 0.15 |
| 30 | A110-1 | Rat | IgG1, ? | 0.34 |
| 31 | G235-2356 | Hamster | IgG | 0.24 |
| 32 | 145-2C11 | Hamster | IgG, gp 1, ? | 0.27 |
| 33 | 31243 | Sheep | IgG | 0.20 |
| 34 | 31127 | Horse | IgG | 0.18 |
| 35 | polyclonal | Horse | IgG | 0.34 |
| 36 | 31229 | Rabbit | F(ab')₂ | 0.19 |
| 37 | 31189 | Rabbit | F(ab')₂ | 0.14 |
| 38 | 31214 | Goat | F(ab')₂ | 0.24 |
| 39 | 31165 | Goat | F(ab')₂ | 0.25 |

*Assay conditions are described in Materials and Methods.
^{H}Mean values of at least two determinations. The background rate of H₂O₂ formation is 0.005 nmol/min in PBS and 0.003 nm/min in PBS with SOD.

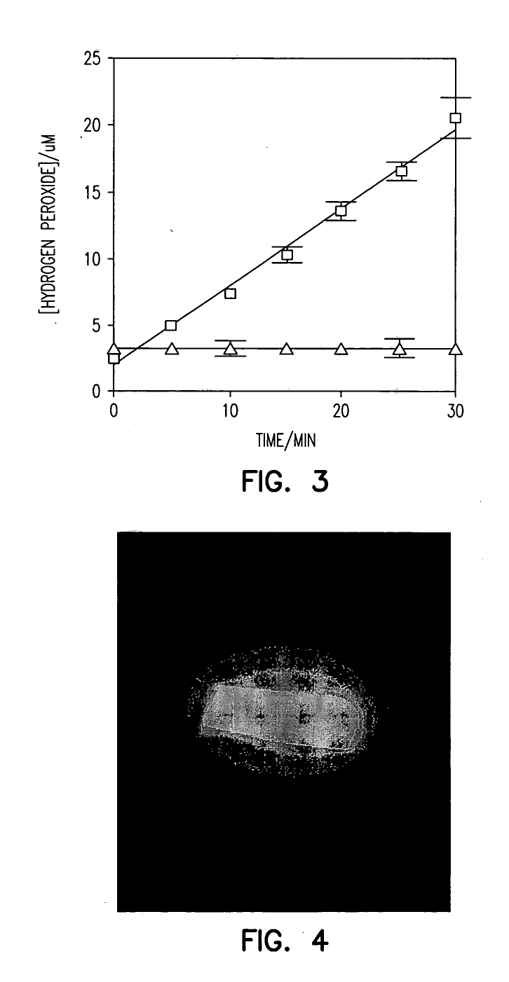

The rates of hydrogen peroxide formation were linear for more than 10% of the reaction, with respect to the oxygen concentration in PBS under ambient conditions (275 μM). With sufficient oxygen availability, the antibodies can generate at least 40 equivalents of H₂O₂ per protein molecule without either a significant reduction in activity or structural fragmentation. An example of the initial time course of hydrogen peroxide formation in the presence or absence of antibody 19G2 is shown in FIG. 3A. This activity is lost following denaturation of the protein by heating.

The data in Table 1 reveal a universal ability of antibodies to generate H₂O₂ from ¹O₂. This function seems to be shared across a range of species and is independent of the heavy and light chain compositions investigated or antigen specificity. The initial rates of hydrogen peroxide formation for the intact antibodies are highly conserved, varying from 0.15 nmol/min/mg [clone A95-1(rat IgG2b)] to 0.97 nmol/min/mg (clone PCP-21H3, a murine monoclonal IgG) across the whole panel. Although the information available is more limited for the component antibody fragments, the activity seems to reside in both the Fab and F(ab′)₂ fragments.

If this activity were due to a contaminant, it would have to be present in every antibody and antibody fragment obtained from diverse sources. However, to further rule out contamination, crystals of the murine antibody 1D4 Fab from which high-resolution x-ray structures have been obtained (at 1.7 D) were investigated for their ability to generate H₂O₂ (FIG. 4). Reduction of ¹O₂ is clearly observed in these crystals.

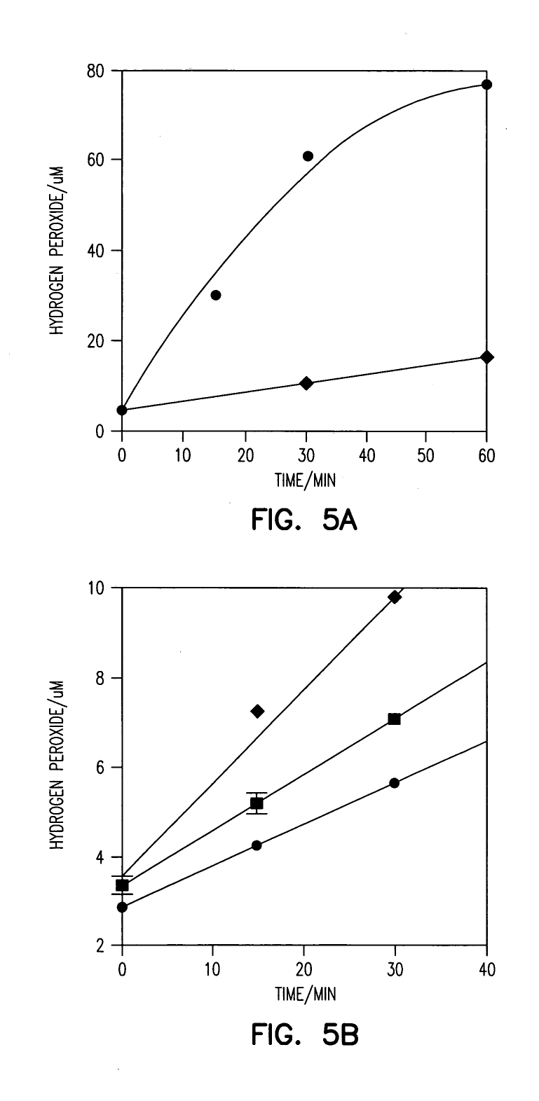

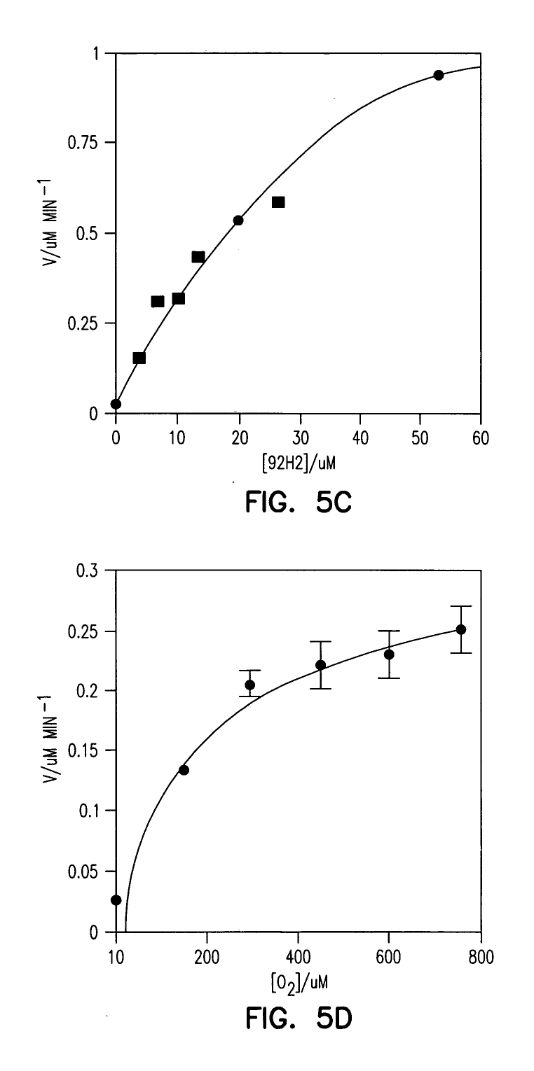

Investigations into this antibody transformation support singlet oxygen as the intermediate being reduced. No formation of hydrogen peroxide occurs with antibodies under anaerobic conditions either in the presence or absence of UV irradiation. Furthermore, no generation of hydrogen peroxide occurs under ambient aerobic conditions without irradiation. Irradiation of antibodies with visible light in the presence of a known photosensitizer of ³O₂ in aqueous solutions hematoporphyrin (HP) (Kreitner, M., Alth, G., Koren, H., Loew, S. & Ebermann, R., Anal. Biochem., 213, 63-67 (1993)), leads to hydrogen peroxide formation (FIG. 5A). The curving in the observed rates is due to consumption of oxygen from within the assay mixture. Concerns that the interaction between photoexcited HP and oxygen may be resulting in O₂^{?}? formation (Beauchamp, C. & Fridovich, I., Anal. Biochem., 44, 276-287 (1971); Srinivasan, V. S., Podolski, D., Westrick, N. J. & Neckers, D. C., J. Am. Chem. Soc., 100, 6513-6515 (1978)) were largely discounted by suitable background experiments with the sensitizer alone (data shown in FIG. 5A). The efficient formation of H₂O₂ with HP and visible light both reaffirm the intermediacy of ¹O₂ and show that UV radiation is not necessary for the Ig to perform this reduction.

Furthermore, incubation of sheep antibody 31243 in the dark at 37EC, with a chemical source of ¹O₂ [the endoperoxide of 3N,3N-(1,4-naphthylidene) dipropionate] leads to hydrogen peroxide formation.

The rate of formation of H₂O₂, by horse IgG with HP (40 μM) in visible light, is increased in the presence of D₂O and reduced with the ¹O₂ quencher NaN₃ (40 mM) (FIG. 5B) (Hasty, N., Merkel, P. B., Radlick, P. & Kearns, D. R. Tetrahedron Lett., 49-52 (1972)). The substitution of D₂O for H₂O is known to promote ¹O₂-mediated processes via an increase of approximately 10-fold in its lifetime (Merkel, P. B., Nillson, R. & Kearns, D. R., J. Am. Chem. Soc., 94, 1030-1031 (1972)).

The rate of hydrogen peroxide formation is proportional to IgG concentration between 0.5 and 20 μM but starts to curve at higher concentrations (FIG. 5C). The lifetime of ¹O₂ in protein solution is expected to be lower than in pure water due to the opportunity for reaction. It is therefore thought that the observed curvature may be due to a reduction in the lifetime of ¹O₂ due to reaction with the antibody.

Significantly, the effect of oxygen concentration on the observed rate of H₂O₂ production shows a significant saturation about 200 μM of oxygen (FIG. 5D). Therefore, the mechanism of reduction may involve either one or more oxygen binding sites within the antibody molecule. By treating the raw rate data to nonlinear regression analysis and by fitting to the Michaelis-Menten equation, a Kₘapp(O₂) of 187 μM and a Vₘₐₓapp of 0.4 nmol/min/mg are obtained. This antibody rate is equivalent to that observed for mitochondrial enzymes that reduce molecular oxygen in vivo.

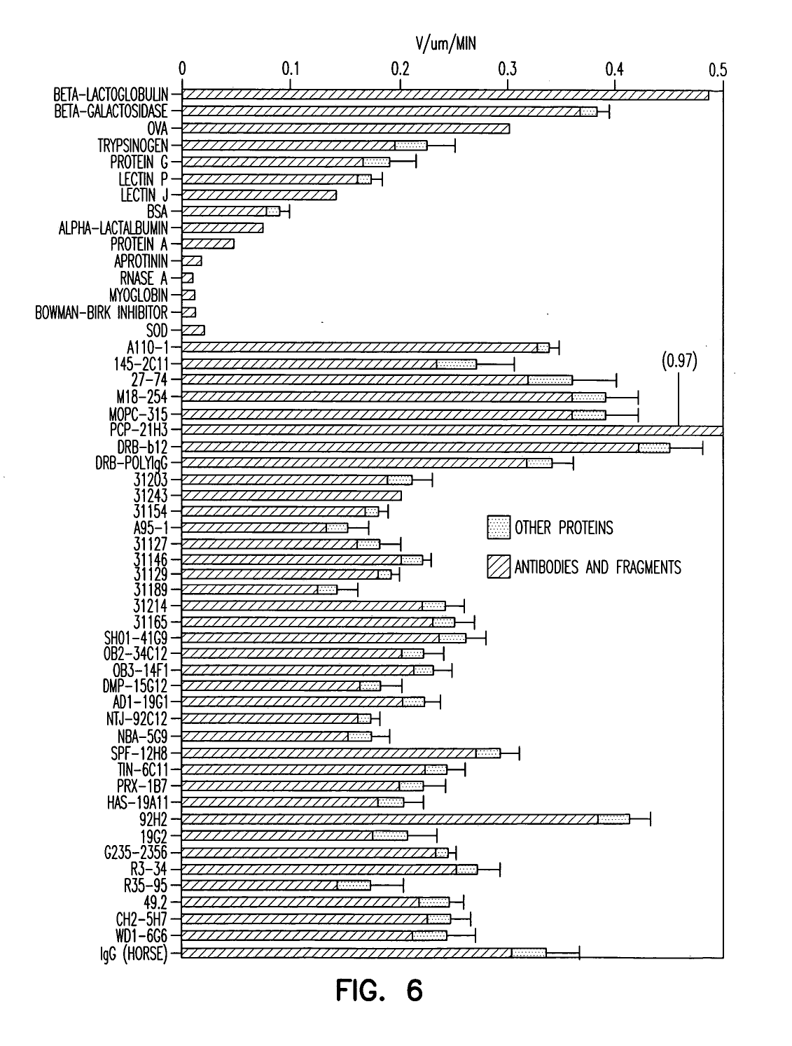

The mechanism by which antibodies reduce ¹O₂ is still being determined. However, the participation of a metal-mediated redox process has been largely discounted because the activity of the antibodies remains unchanged after exhaustive dialysis in PBS containing EDTA (4 mM). This leaves the intrinsic ability of the amino acid composition of the antibodies themselves. Aromatic amino acids such as tryptophan (Trp) can be oxidized by ¹O₂ via electron transfer (Grossweiner, L. I., Curr. Top. Radiat. Res. Q., 11, 141-199 (1976)). In addition, disulfides are sufficiently electron rich that they can also be oxidized (Bent. D. V. & Hayon, E., J. Am. Chem. Soc., 87, 2612-2619 (1975)). Therefore, there is the potential that Trp residues and/or the intrachain or interchain disulfide bonds homologous to all antibodies are responsible for ¹O₂ reduction. To both investigate to what extent this ability of antibodies is shared by other proteins and to probe the mechanism of reduction, a panel of other proteins was studied (FIG. 6).

Whereas other proteins can convert ¹O₂ into O₂^{?}?, in contrast to antibodies it is by no means a universal property. RNase A and superoxide dismutase, which do not possess Trp residues but have several disulfide bonds, do not reduce ¹O₂. Similarly, the Bowman-Birk inhibitor protein (Voss, R.-H., Ermler, U., Essen, L.-O., Wenzl, G., Kim, Y.-M. & Flecker, P., Eur. J. Biochem., 242, 122-131 (1996); Baek, J. & Kim, S., Plant Physiol., 102, 687 (1993)) that has seven disulfide bonds and zero Trp residues does not reduce ¹O₂. In contrast, chick ovalbumin, which has only 2 Trp residues (Feldhoff, R. & Peters, T. J., Biochem. J., 159, 529-533 (1976)), is one of the most efficient proteins at reducing ¹O₂.

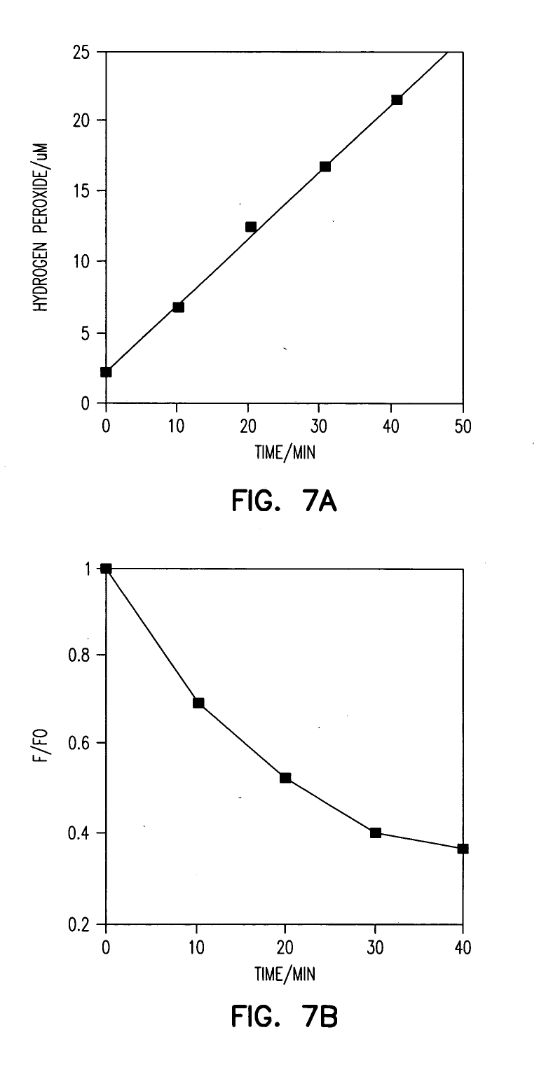

Given the loss of antibody activity upon denaturation, the location of key residues in the protein is likely to be more critical than their absolute number. Because the majority of aromatic residues in proteins are generally buried to facilitate structural stability (Burley, S. K. & Petsko, G. A., Science, 229, 23-28 (1985)), the nature of the reduction process was explored in terms of relative contribution of surface and buried residues by fluorescence-quenching experiments. Aromatic amino acids in proteins are modified by the absorption of ultraviolet light, especially in the presence of sensitizing agents such as molecular oxygen or ozone (Foote, C. S., Science, 162, 963-970 (1968); Foote, C. S., Free Radicals Biol., 2, 85-133 (1976); Gollnick, K., Adv. Photochem., 6, 1-122 (1968)). Trp reacts with ¹O₂ via a [2+2] cycloaddition to generale N-formylkynurenine or kynurenine, which are both known to significantly quench the emission of buried Trp residues (Mach, H., Burke, C. J., Sanyal, G., Tsai, P.-K, Volkin, D. B. & Middaugh, C. R. in Formulation and Delivery of Proteins and Peptides, eds. Cleland, J. L. & Langer, R. (American Chemical Society, Denver, Colo.) (1994)). The intrinsic fluorescence of horse IgG is rapidly quenched to 30% of its original value during a 40-min irradiation, whereas hydrogen peroxide generation is linear throughout (r²=0.998) (FIG. 7). If the reduction of singlet oxygen is due to antibody Trp residues, then the solvent-exposed Trp seem to contribute to a lesser degree than the buried ones. This factor may help to explain why this ability is so highly conserved among antibodies. In greater than 99% of known antibodies there are two conserved Trp residues, and they are both deeply buried: Trp-36 and Trp-47 (Kabat, E. A., Wu, T. T., Perry, H. M., Gottesman, K. S. & Foeller, C., Sequences of Proteins of Immunological Interest (U.S. Department of Health and Human Services, Public Health Service, National Institutes of Health, Bethesda, Md.) (1991)).

Throughout nature, organisms have defended themselves by production of relatively simple chemicals. At the level of single molecules, this mechanism has thought to be largely abandoned with the appearance in vertebrates of the immune system. It was considered that once a targeting device had evolved, the killing mechanism moved elsewhere. The present results realign recognition with killing within the same molecule. In a certain sense this chemical immune system parallels the purely chemical defense mechanism of lower organisms, with the exception that a more sophisticated and diverse targeting element is added.

Given the constraints that an ideal killing system must use host molecules in a localized fashion while minimizing self damage, one can hardly imagine a more judicious choice than ¹O₂. Because one already has such a reactive molecule, it is important to ask what might be the advantage of its further conversion by the antibody.

The key issue is that by conversion of the transient singlet oxygen molecule (lifetime 4 μs) into the more stable O₂^{?}?, one now has access to hydrogen peroxide and all of the toxic products it can generate. In addition, superoxide is the only molecular oxygen equivalent remaining at the end of the oxygen-scavenging cascade. Therefore, this “recycling” may serve as a crucial mechanism for potentiation of the microbicidal process. Another benefit of singlet molecular oxygen is that it is only present when the host is under assault, thereby making it an “event-triggered” substrate. Also, because there are alternative ways to defend that use accessory systems, this chemical arm of the immune system might be silent under many circumstances. This said, however, there may be many disease states where antibody and singlet oxygen find themselves juxtaposed, thereby leading to cellular and tissue damage. Given that diverse events in man lead to the production of singlet oxygen, its activation by antibodies may lead to a variety of diseases ranging from autoimmunity to reperfusion injury and atherosclerosis (Skepper et al., Microsc. Res. Tech., 42, 369-385 (1998)).

### Example II

### Antibodies Catalyze the Oxidation of Water Methods and Materials

Crystallography: IgG 4C6 was digested with papain and the Fab′ fragment purified using standard protocols (Harlow and Lane). The Fab′ was crystallized from 13-18% PEG 8 K, 0.2 M ZnAc, 0.1 M cacodylate, pH 6.5. Crystals were pressurized under xenon gas at 200 psi for two minutes (Soltis et al., J. Appl. Cryst., 30, 190, (1997)) and then flash cooled in liquid nitrogen. Data were collected to 2.0 A resolution at SSRL BL9-2. The structure was solved by molecular replacement using coordinates from the native 4C6 structure, and xenon atom sites were identified from strong peaks in the difference Fourier map. Refinement of the structure was done in CNS (Brünger et al., Acta. Crystallogr., D54, 905 (1998)) to final R=23.1% and R_{free}=25.7%. The occupancies of the two xenon atoms were refined after fixing their B values fifty percent higher than the B factors of the immediately surrounding protein. The figures were generated in Bobscript (R. M. Esnouf, Acta Crystallog., D55, 938 (1999)).

Scanning of the Kabat database: The Kabat database of human and mouse sequences was analyzed to determine the number of Trp, Tyr, Cys, Met in their structures. Sequences were rejected if there were too many residue deletions or missing fragments. This allowed a high certainty analysis for 2068 of the 3894 sequences available. The values are reported as the mean totals with the range in parentheses of the C_{H}, V_{H}, C_{L} and V_{L} (? and ?.) regions: Trp 15.5 (14 to 31), Tyr 30.4 (13 to 47), Cys 19.3 (15 to 29), Met 11.6 (7 to 32), His 13.3 (8 to 28). Grand total=90.1 (49 to 167).

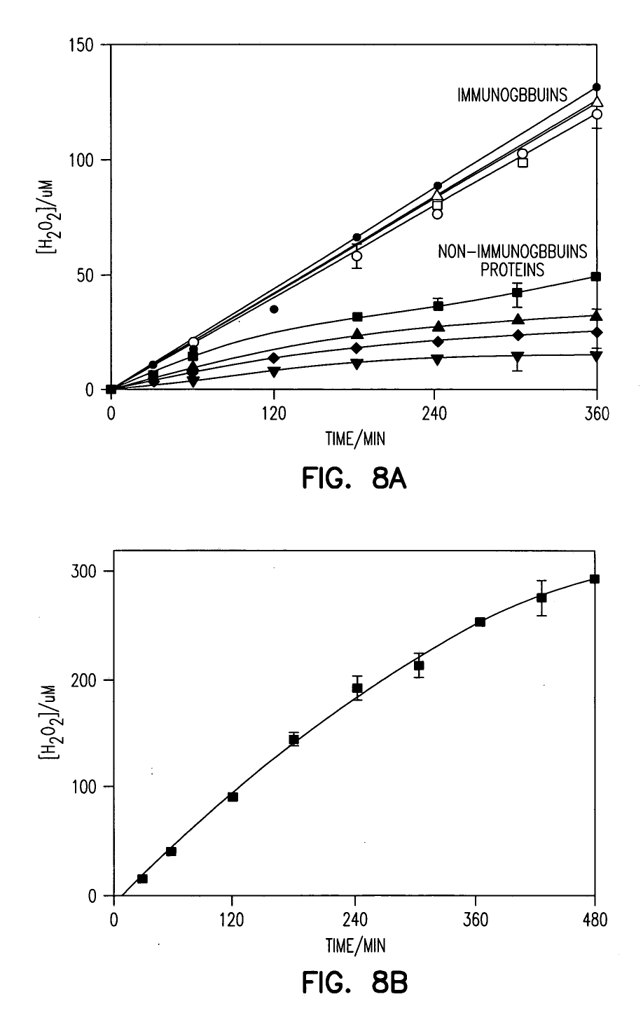

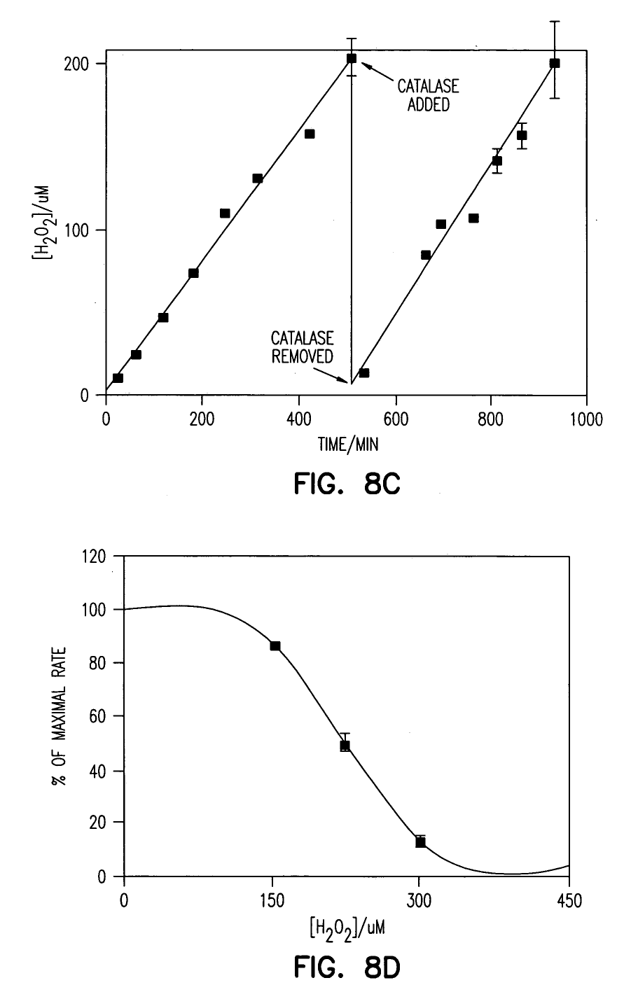

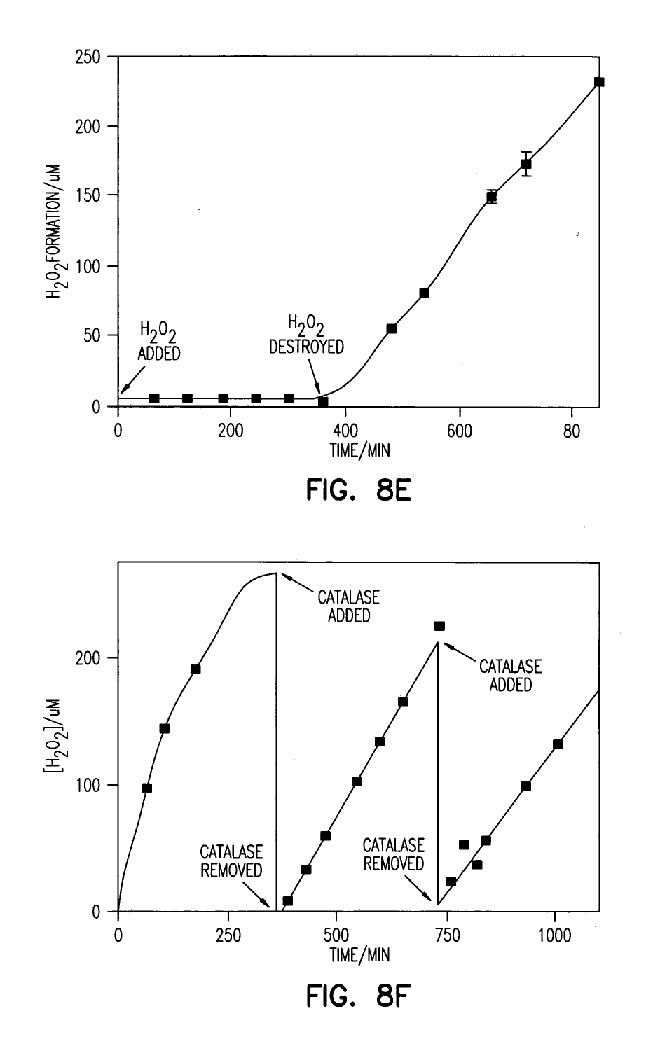

Inductively coupled plasma atomic emission spectroscopy: Inductively coupled plasma atomic emission spectroscopy (ICP-AES) of mAb PCP21H3 was performed on a Varian, Axial Vista Simultaneous ICP-AES spectrometer. Mouse monoclonal antibody (PCP21H3) was exhaustively dialyzed into sodium phosphate buffered saline (PBS, 50 mM pH 7.4) with 20 mM EDTA. In a typical assay 300 μL of a 10.5% HNO₃ solution was added to 100 μL of a 10 mg/mL antibody solution and was incubated at 70° C. for 14 hours. This solution was then diluted to 2 mL with MQH₂O and then analyzed by comparison to standards. ICP-AES analysis results are reported in parts per million (μg/mL): Ag 0.0026 (0.0072 atoms per antibody molecule); Al 0.0098 (0.113 atoms per antibody molecule); As 0.0062 (0.025 atoms per antibody molecule); Ba below level of detection; Ca 0.0355 (0.266 atoms per antibody molecule). The high Ca concentration is a result of contamination of the phosphate buffer system utilized in our assay system. To investigate the effect of Ca(II) on the rate of antibody-mediated H₂O₂, the irradiation of antibody samples was performed using the assay procedure outlined in the legend of FIG. 8A with the addition of varying concentrations of CaCl₂ (0-100 μM). The process was found to be independent of Ca(II) concentration; Cd 0.0007 (0.0187 atoms per antibody molecule); Ce 0.0012 (0.003 atoms per antibody molecule); Co 0.0013 (0.007 atoms per antibody molecule); Cr 0.0010 (0.006 atoms per antibody molecule); Cu 0.0014 (0.007 atoms per antibody molecule); Fe 0.0089 (0.048 atoms per antibody molecule); Gd 0.0008 (0.001 atoms per antibody molecule); K 0.0394 (0.302 atoms per antibody molecule); La 0.0007 (0.002 atoms per antibody molecule); Li 0.0013 (0.056 atoms per antibody molecule); Mg 0.0027 (0.033 atoms per antibody molecule); Mn 0.0007 (0.004 atoms per antibody molecule); Mo 0.0023 (0.007 atoms per antibody molecule); Na 102.0428 (1332 atoms per antibody molecule); Ni 0.0007 (0.004 atoms per antibody molecule); P 14.3521(138.9 atoms per antibody molecule); Pb below level of detection; Rb 0.0007 (0.002 atoms per antibody molecule); Se below level of detection; V 0.0109 (0.019 atoms per antibody molecule); W 0.0119 (0.019 atoms per antibody molecule); Zn 0.0087 (0.040 atoms per antibody molecule).

Oxygen isotope experiments: In a typical experiment, a solution of antibody (6.7 μM, 100 μL) or non-immunoglobulin protein (50 μM, 100 μL) in PB (160 mM phosphate; pH 7.4) was lyophilized to dryness and then dissolved in H₂O₂ (100 μL, 98%). Sodium chloride was excluded to minimize signal suppression in the MS. The higher concentration of non-immunoglobulin protein was necessary to generate a detectable amount of H₂O₂ for the MS assay. This protein solution was irradiated on a UV-transilluminator under saturating ¹⁶O₂ aerobic conditions in a sealed quartz cuvette for 8 hours at 20EC. The H₂O₂ concentration was determined after 8 hours using the Amplex Red assay (Zhou et al., Anal. Biochem., 253, 162 (1997)). The sample was then filtered by centrifugation through a microcon (size-exclusion filter) to remove the protein and the H₂O₂ concentration re-measured. TCEP (freshly prepared 20 mM stock in H₂¹⁸O) was added (ca. 2 mol eq relative to H₂O₂) and the solution was left to stand at 37EC for 15 minutes, after which time all the H₂O₂ had reacted. The TCEP solution in H₂¹⁸O was prepared fresh prior to every assay because ¹⁸O is slowly incorporated into the carboxylic acids of TCEP (over days). During the time course of the assay, no incorporation of ¹⁸O occurs due to this pathway. Furthermore, there is no incorporation of ¹⁸O from H₂¹⁸O into the ¹⁶O phosphine oxide. The peak at 249 m/z is the (M−H)⁻ of TCEP. The peak at 249 is observed in all the MS because an excess of TCEP (twofold) relative to H₂O₂ is used in the assay.

The reproducibility of the ¹⁶O/¹⁸O ratio from protein samples lyophilized together is reasonable (±10%). However, problems with removing protein-bound water molecules during the lyophilization process means that the observed ratios can vary between samples from different lyophilization batches by as much as 2:1 to 4:1 (when lyophilizing from H₂¹⁶O). It is, therefore, important that rigorous lyophilization and degassing procedures are followed. In this regard, the ¹⁸O₂ and H₂¹⁶O experiments exhibit far less inter-assay variability due to the relative ease of removing protein-bound oxygen molecules.

Antibodies from different species give similar ratios within the experimental constraints detailed below: ¹⁶O:¹⁸O: WD1-6G6 mIgG (murine) 2.1:1; poly-IgG (horse) 2.2:1; poly-IgG(sheep) 2.2:1; EP2-19G2 mIgG (murine) 2.1:1; CH2-5H7 mIgG (murine) 2.0:1; poly-IgG (human) 2.1:1. Ratios are based on the mean value of duplicate determinations except for poly-IgG (horse), which is the mean value of ten measurements. All assays and conditions are as described above.

In a typical experiment, a solution of sheep or horse poly-IgG (6.7 μM, 100 μL) in PB (160 mM phosphate; pH 7.4) was degassed under an argon atmosphere for 30 min. This solution was then saturated with ¹⁸O₂ (90%) and irradiated as described above. Assays and procedures are then as described herein.

Assay for H₂O₂ production as a function of the efficiency of ¹O₂ formation via ³O₂ sensitization with hematoporphyrin IX: The assay is a modification of a procedure developed by H. Sakai and co-workers, Proc. SPIE-Int. Soc. Opt. Eng., 2371, 264 (1995). In brief, the horse poly-IgG (1 mg/mL) in PBS (50 mM, pH 7.4) and hematoporphyrin IX (40 μM) is irradiated with white light from a transilluminator. Aliquots are removed (50 μL) and the concentration of H₂O₂ and 3-aminophthalic acid measured simultaneously. H₂O₂ concentration was measured by the amplex red assay (Zhou et al., Anal. Biochem., 253, 162 (1997)). 3-Aminophthalic acid concentration was measured by reversed-phase HPLC on a Hitachi D4000 series machine with an Adsorbosphere-C18 column, a UV detector at 254 nm, and a mobile phase of acetonitrile/water (0.1% TFA) of 18:82 at 1 mL/min (retention time of luminol=7.4 min and 3-aminophthalic acid 3.5 min). The concentrations of luminol and 3-aminophthalic acid were determined by comparison of peak height and area to control samples. The experimental data yields the amount of ¹O₂ formed by hematoporphyrin IX (being directly proportional to the amount of 3-aminophthalic acid formed) and the amount of H₂O₂ formed by the antibody. N.B. There is no significant amount of ¹O₂ formed by antibodies without hematoporphyrin IX in white light.

Any concerns that the amplex red assay may be detecting protein-hydroperoxide derivatives in addition to H₂O₂ have been discounted because the apparent H₂O₂ concentration measured using this method is independent of whether irradiated protein is removed from the sample (by size-exclusion filtration).

Quantum Chemical Methods: All QC calculations were carried out with Jaguar [Jaguar 4.0, Schrödinger, Inc. Portland, Oreg., 1998. See B. H. Greeley, T. V. Russo, D. T. Mainz, R. A. Friesner, J.-M. Langlois, W. A. Goddard III, R. E. Donnelly, J. Chem. Phys., 101, 4028 (1994)] using the B3LYP flavor of density functional theory (DFT) [J. C. Slater in Quantum Theory of Molecules and Solids, Vol. 4: The Self-Consistent Field of Molecules and Solids, McGraw Hill, New York, (1974)], that includes the generalized gradient approximation and exact exchange. The 6-31G** basis set was used on all atoms. All geometries were fully optimized. Vibrational frequencies were calculated to ensure that each minimum is a true local minimum (only positive frequencies) and that each transition state (TS) has only a single imaginary frequency (negative eigenvalue of the Hessian). Such QC calculations have been demonstrated to have an accuracy of ˜3 kcal/mol for simple organic molecules. Non-closed shell molecules such as O₂ and ³O₂ are expected to have larger errors. However, such errors are expected to be systematic such that the mechanistic implications of the QC results should be correct. All energetics are reported in kcal/mol without correcting for zero point energy or temperature.

### Results and Discussion

Antibodies are capable of generating hydrogen peroxide (H₂O₂) from singlet molecular oxygen (¹O₂). However, it was not known until now, as reported herein, that the process was catalytic and the source of electrons. It is now shown that antibodies are unique as a class of proteins in that they can produce up to 500 mole equivalents of H₂O₂ from ¹O₂, without a reduction in rate, in the absence of any discernible cofactor and electron donor. Based on isotope incorporation experiments and kinetic data, it is proposed that antibodies are capable of facilitating an unprecedented addition of H₂O to ¹O₂ to form H₂O₃ as the first intermediate in a reaction cascade that eventually leads to H₂O₂. X-ray crystallographic studies with xenon point to conserved oxygen binding sites within the antibody fold where this chemistry could be initiated. This findings suggest a unique protective function of immunoglobulins against ¹O₂ and raise the question of whether the need to detoxify ¹O₂ has played a decisive role in the evolution of the immunoglobulin fold.

Antibodies, regardless of source or antigenic specificity, generate hydrogen peroxide (H₂O₂) from singlet molecular oxygen (¹O₂) thereby potentially aligning recognition and killing within the same molecule (Wentworth et al., Proc. Natl. Acad. Sci. U.S.A., 97, 10930 (2000)). Given the potential chemical and biological significance of this discovery, the mechanistic basis and structural location within the antibody of this process has been investigated. These combined studies reveal that, in contrast to other proteins, antibodies may catalyze an unprecedented set of chemical reactions between water and singlet oxygen.

Kinetic studies. Long term UV irradiation studies reveal that antibody-mediated H₂O₂ production is a much more efficient process than is the case for the non-immunoglobulin proteins (FIG. 8A). Typically antibodies exhibit linearity in H₂O₂ formation for up to 40 mole equivalents of H₂O₂ before the rate begins to decline asymptotically (FIG. 8B). By contrast, non-immunoglobulin proteins display a short ‘burst’ of H₂O₂ production followed by quenching as photo-oxidation occurs (FIG. 8A).

In contrast to other proteins, antibodies are able to resume photo-production of H₂O₂ at the same initial rate as at the start of the experiment if the H₂O₂ generated during the assay is removed by catalase, as shown for murine monoclonal IgG PCP21H3 (FIG. 8C). This profile of continued linear production of H₂O₂ after catalase-mediated destruction of H₂O₂ was conserved for all antibodies assayed. Thus, the H₂O₂ that accumulates during the process is inhibiting (reversibly) its own formation. The apparent IC₅₀ was estimated as 225 μM (FIG. 8D). Inhibition of the catalytic function of an enzyme either by substrates, transition state analogs or reaction products is often taken as strong evidence for an active site phenomenon. It has already been noted that the antibody-mediated photo-production of H₂O₂ is saturable with molecular oxygen (Kₘapp(O₂ 187 μM) (Wentworth et al., Proc. Natl. Acad. Sci. U.S.A., 97, 10930 (2000)). This formal product inhibition of H₂O₂ provides further evidence for such a binding site phenomenon.

An earlier report concerning the photo-production of H₂O₂ by antibodies did not probe the maximum amount of H₂O₂ that could be generated (Wentworth et al., Proc. Natl. Acad. Sci. U.S.A., 97, 10930 (2000)). This issue has been examined by repetitive cycles of UV irradiation of antibody samples followed by removal of the generated H₂O₂ by catalase (FIG. 8C shows two such cycles). In one series of experiments, the cycle of UV-irradiation and addition of catalase was carried out for up to 10 cycles (horse poly IgG in PBS, pH 7.4). During these experiments >500 mole equivalents (equiv.) of H₂O₂ were generated, with only a slight reduction in the initial rate being observed. Beside antibodies, the only other protein that was found thus far to generate H₂O₂ in such an efficient and long-term manner was the aB T cell receptor (aB TCR) (FIG. 8F). Interestingly, the aB TCR shares a similar arrangement of its immunoglobulin fold domains with antibodies (Garcia et al., Science, 274, 209 (1996)). However, possession of this structural motif seems not necessarily to confer an H₂O₂-generating ability on proteins as demonstrated by β₂-microglobulin, which does not generate H₂O₂ even though it is a member of the immunoglobulin superfamily (Welinder et al., Mol. Immunol., 28 177 (1991)).

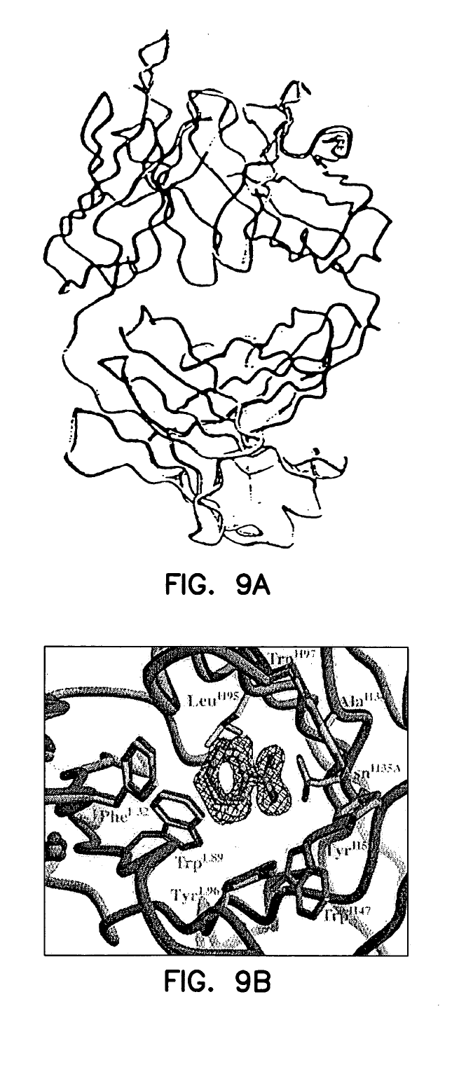

The antibody structure is remarkably inert against the oxidizing effects of H₂O₂. When exposed to standard UV irradiation conditions for 6 hours in the presence of H₂O₂ (at a concentration high enough to fully inhibit H₂O₂ production), a polyclonal horse IgG antibody sample becomes fully active once the inhibitory H₂O₂ has been destroyed by catalase (FIG. 8E). The ability to continue H₂O₂ production for long periods at a constant rate, even after exposure to H₂O₂, reveals a remarkable, and hitherto unnoticed, resistance of the antibody structural fold to both chemical and photo-oxidative modifications suffered by other proteins. SDS-PAGE gel analysis of antibody samples after UV irradiation under standard conditions for 8 hours reveals neither significant fragmentation nor agglomeration of the antibody molecule. To ensure that there was no change in the protein structure in the presence of H₂O₂ (that may be contributing to the apparent inhibitory effect of H₂O₂) even at the level of side-chain position, x-ray crystal structures of Fab 4C6 were determined in the presence and absence of H₂O₂. Fab 4C6 was selected because its native crystals diffract to a higher resolution than any other published antibody (˜1.3 D). The root mean square difference (RMSD) of key structural parameters were compared for the 4C6 structure before and after a soak experiment with 3 mM H₂O₂. RMSD of all atoms=0.412 D, RMSD Ca atoms=0.327 D, RMSD main chain atoms=0.328 D, RMSD side-chain atoms=0.488 D. The overlayed native and H₂O₂-treated structures of murine Fab 4C6 (Li et al., J. Am. Chem. Soc., 117, 3308 (1995)) are superimposable, reinforcing the evidence of stability of the antibody fold to H₂O₂ (FIG. 9).

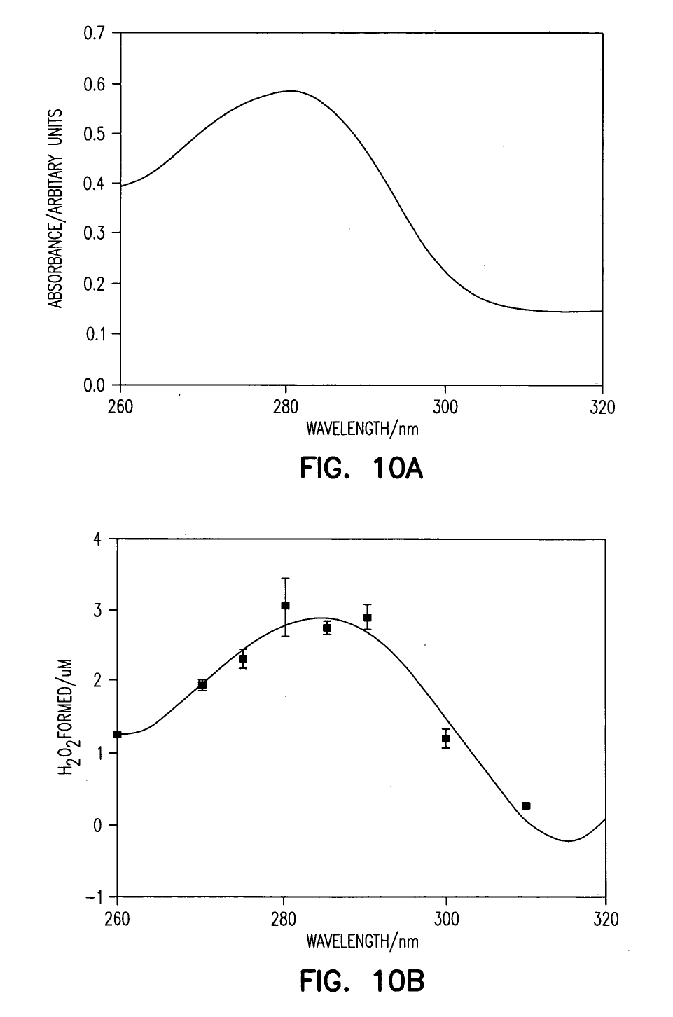

An action spectrum of the antibody-mediated photo-production of H₂O₂ and the corresponding absorbance spectrum of the antibody protein for the same wavelength range (260-320 nm) are juxtaposed in FIG. 10. The two spectra are virtually superimposable with maximal efficiency of H₂O₂ production being observed at an excitation wavelength that coincides with the UV absorbance maxima of tryptophan in proteins.

Probing the efficiency of H₂O₂ production by horse IgG as a function of the efficiency of ¹O₂ formation via ³O₂ sensitization with hematoporphyrin IX (f A=0.22 in phosphate buffer pH 7.0 and visible light reveals that for every 275″ 25 mole equivalents of ¹O₂ generated by sensitization, 1 mole equivalent of H₂O₂ is generated by the antibody molecule (Wilkinson et al., J. Phys. Chem. Ref. Data, 22, 113 (1993); Sakai et al., Proc. SPIE-Int. Soc. Opt. Eng., 2371, 264 (1995)).

The question of the electron source. The mechanism problem posed by the antibody-mediated H₂O₂ production from singlet oxygen has to be sharply divided into two sub-problems: one referring to the electron source for the process and the other concerning the chemical mechanism of the process. Given that the conversion of ¹O₂ to H₂O₂ requires two mole equivalents electrons, the fact that antibodies can generate >500 equivalents of H₂O₂ per equivalent of antibody molecule raises an acute electron inventory problem. The search for this electron source began with the most distinct possibilities. Since electron transfer through proteins can occur with remarkable facility and over notably large distances (Winkler et al., Pure & Appl. Chem., 71, 1753 (1999); Winkler, Curr. Opin. Chem. Biol., 4, 192 (2000)), the first considered was that a collection of the residues implicated as electron donors cited in normal protein photo-oxidation processes might be involved. The nearly constant rate of H₂O₂ production by antibodies and the aβ-TCR during the repetitive cycles of irradiation and catalase treatment (FIGS. 8C and 8E) argued against such a mechanism because a marked reduction of rate would have to accompany H₂O₂ production as the residues capable of being oxidized become exhausted. This reduction of rate would be further exacerbated because the redox potentials of the remaining unoxidized residues would have to rise as the protein becomes more positively charged.

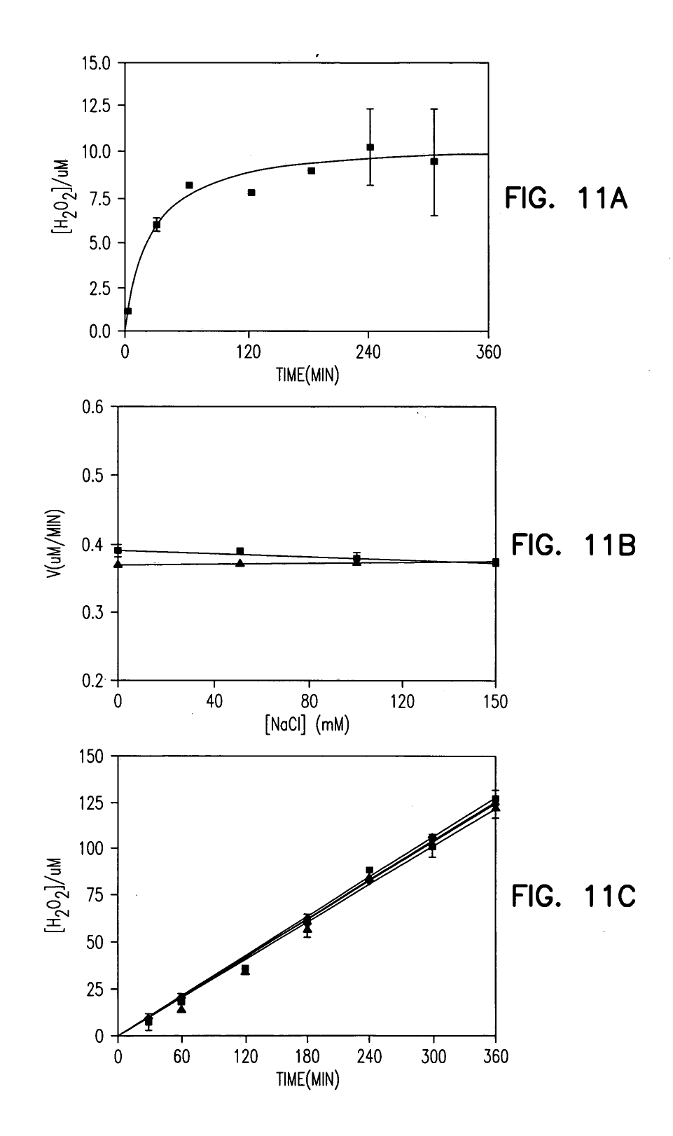

Normal protein photo-oxidation is a complex cascade of processes that leads to the generation of ¹O₂ and other reactive oxygen species (ROS), such as superoxide anion (O₂^{?}?, peroxyl radical (HO₂?) and H₂O₂ (Foote, Science, 162 963 (1968)). Present mechanistic thinking links the sensitivity of proteins to photo-oxidation with up to five amino acids: tryptophan (Trp), tyrosine (Tyr), cysteine (and cystine), methionine (Met), and histidine (His) (Straight and Spikes, in Singlet O₂, A. A. Frimer, Ed. (CRC Press, Inc., Boca Raton, Fla., 1985), vol IV9, pp. 91-143; Michaeli and Feitelson, Photochem. Photobiol., 59, 284 (1994)). The photo-production of H₂O₂ by Trp and molecular oxygen is a well-characterized process that involves, at least in part, the formation and reduction of ¹O₂ to O₂^{?} that spontaneously dismutates into H₂O₂ and ³O₂ (McMormick and Thompson, J. Am. Chem. Soc., 100, 312 (1978)). Tryptophan, both as an individual amino-acid and as a constituent of proteins, is particularly sensitive to near-UV irradiation (300-375 nm) under aerobic conditions, owing to its conversion to NN-formylkynurenine (NFK) that is a particularly effective near-UV (?ₘₐₓ 320 nm) photosensitizer (Walrant and Santus, Photochem. Photobiol., 19, 411 (1974)). However, Trp photo-oxidation is accompanied by sub-stoichiometric production of H₂O₂ (ca. 0.5 mole equivalents) during near-UV irradiation (FIG. 11A) (McMormick and Thompson, J. Am. Chem. Soc., 100, 312 (1978)) and the most efficient non-immunoglobulin protein at H₂O₂ photo-production, β-galactosidase, generates only 5.9 mol eq. of H₂O₂ from its 39 Trp residues (FIG. 8A) (Fowler and Zabin, J. Biol. Chem., 253 5521 (1978)).

Scanning of the Kabat database of human and mouse antibody heavy- and light-chain sequences (2068 of 3894 sequences were analyzed) revealed that antibodies rarely have more than 15 Trp residues in their entire structure (mean value=15.5 with a range of 14 to 31 Trp residues)(Kabat et al., Sequences of Proteins of Immunological Interest (US Department of Health and Human Services, Public Health Service, NIH, ed. 5th, 1991); Martin, PROTEINS: Struct., Funct. and Genet., 25, 130 (1996)). In fact, even if all of the amino acids that are implicated in protein photo-oxidation processes vide supra are collectively involved in antibody-mediated H₂O₂-production, there is still an insufficient number of these residues (mean value=90.1 with a range of 49 to 167 reactive residues) to account for the 500 mole equivalents of H₂O₂ generated.

The potential of chloride ion (present at 150 mM in PBS) as a reducing equivalent was then investigated given that chloride ion is known to be a suitable electron source for photo-production of H₂O₂ via a triplet excited state of an anthraquinone (Scharf and Weitz, Symp. Quantum Chem. Biochem., Jerusalem vol. 12 (Catal. Chem. Biochem.: Theory Exp.), pp. 355-365 (1979)). This possibility was quickly discounted when the rate of H₂O₂ production by immunoglobulins was found to be independent of chloride ion concentration (FIG. 11B).

The possible role of metal ions was investigated. While such ions could hardly be present in antibodies in such amounts that they could serve as an electron source, trace amounts of them might play a central role as catalytic redox centers. Experiments were performed that, for all practical purposes, allow the implication of trace metals in this process to be ruled out. The rate of antibody-mediated photo-production of H₂O₂ is unchanged before and after exhaustive dialysis of antibody samples with EDTA-containing buffer (FIG. 11C). After EDTA treatment of antibody samples, ICP-atomic emission spectroscopy (AES) revealed the presence of trace metal ions remaining in amounts that are far below parts per million. For a trace metal to be implicated in this reaction it must be common to all antibodies because all antibodies assayed have this intrinsic ability. It is generally accepted that metal-binding is not an implicit feature of antibodies and is consistent with our own analysis of antibody crystals as well as the approximate 300 antibody structures available on the Brookhaven database.

All of the observations thus far forcibly pointed towards the need to identify an electron source that would not imply a deactivation of the protein catalyst and that could account for the high turnover numbers and hence, for a quasi unlimited source of electrons. A more broad consideration of the chemical potential of ¹O₂ was done. The participation of this energized form of molecular oxygen in the antibody-mediated mechanism was clearly inferred from a previous report (Wentworth et al., Proc. Natl. Acad. Sci. U.S.A., 97, 10930 (2000)). In brief, the antibody-mediated rate of H₂O₂ photo-production is increased in D₂O and reduced in the presence of the ¹O₂ quencher, sodium azide. Furthermore, antibodies have been shown to generate H₂O₂ via sensitization of ³O₂ with hematoporphyrin IX in visible light, and in the dark with the endoperoxide of disodium 3N,3N-(1,4-naphthylidene) dipropionate (a chemical ¹O₂ source). The involvement of ¹O₂ is also in line with the close similarity of the action spectrum of antibody-mediated H₂O₂ production and the absorbance spectrum of antibody constituent tryptophans (FIG. 10).

Given that the known chemistry of ¹O₂ can be conceptualized as the chemistry of the super-electrophile “dioxa-ethene” (Foote, Acc. Chem. Res., 1, 104 (1968), the heretofore unknown possibility was considered that a molecule of water may, in the presence of an antibody, add as a nucleophile to ¹O₂ and form H₂O₃ as an intermediate. Thus, water becoming oxidized to H₂O₂ would fulfill the role of the electron source.

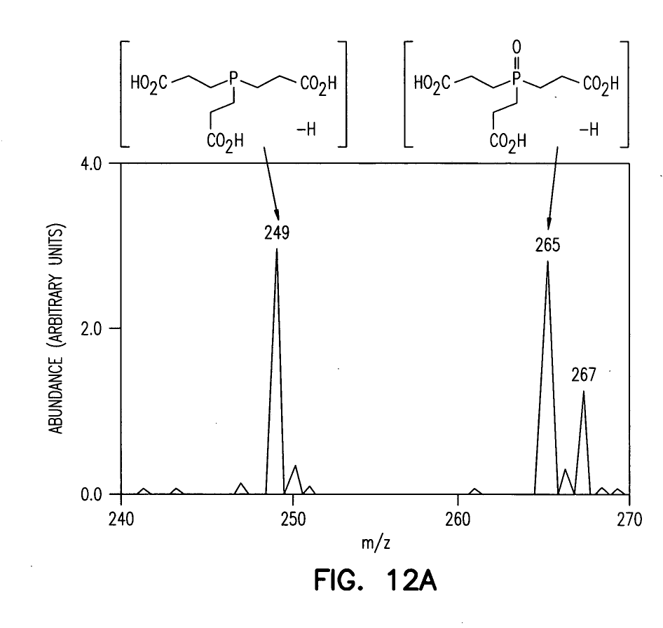

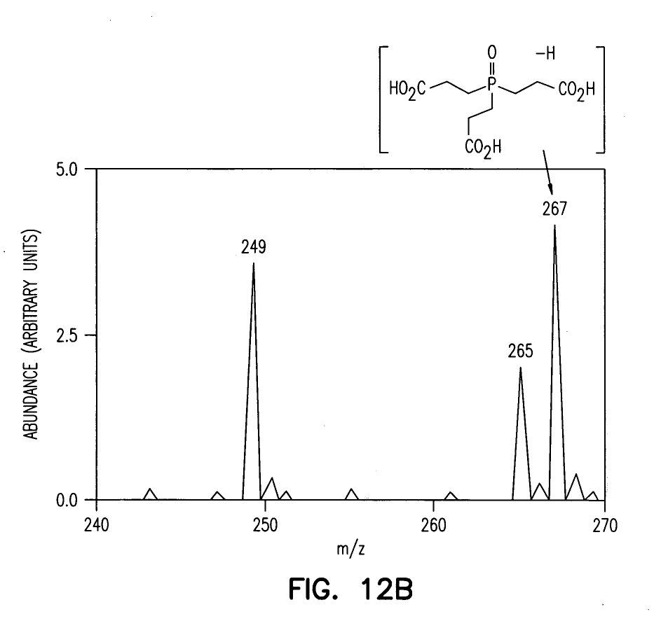

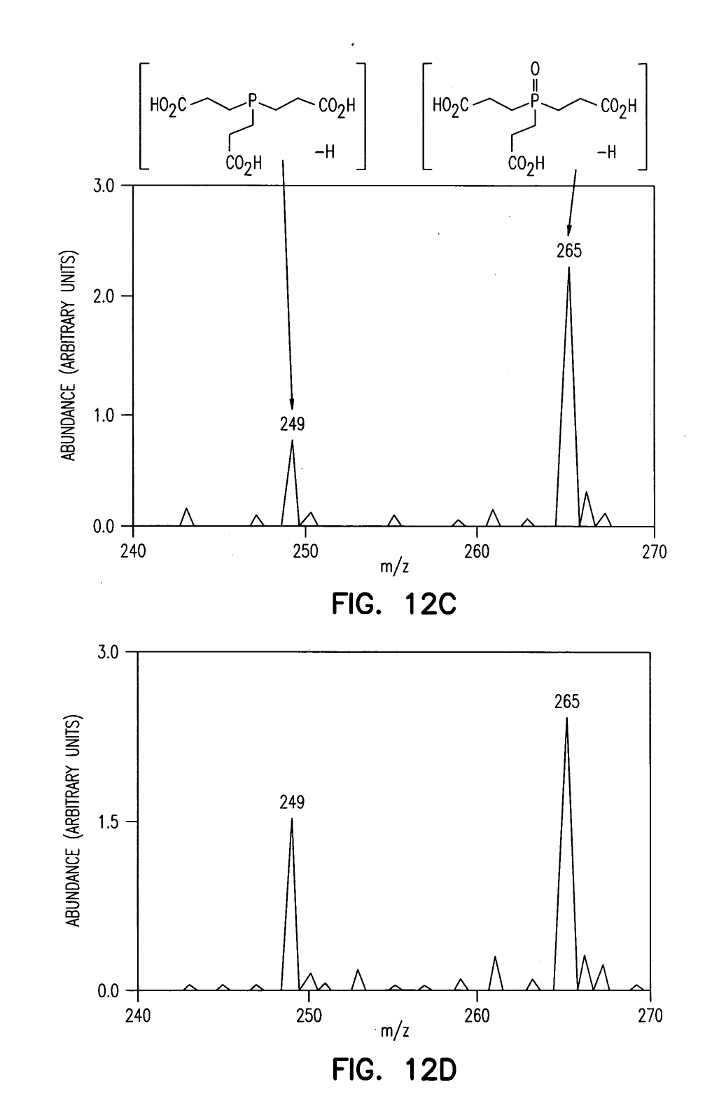

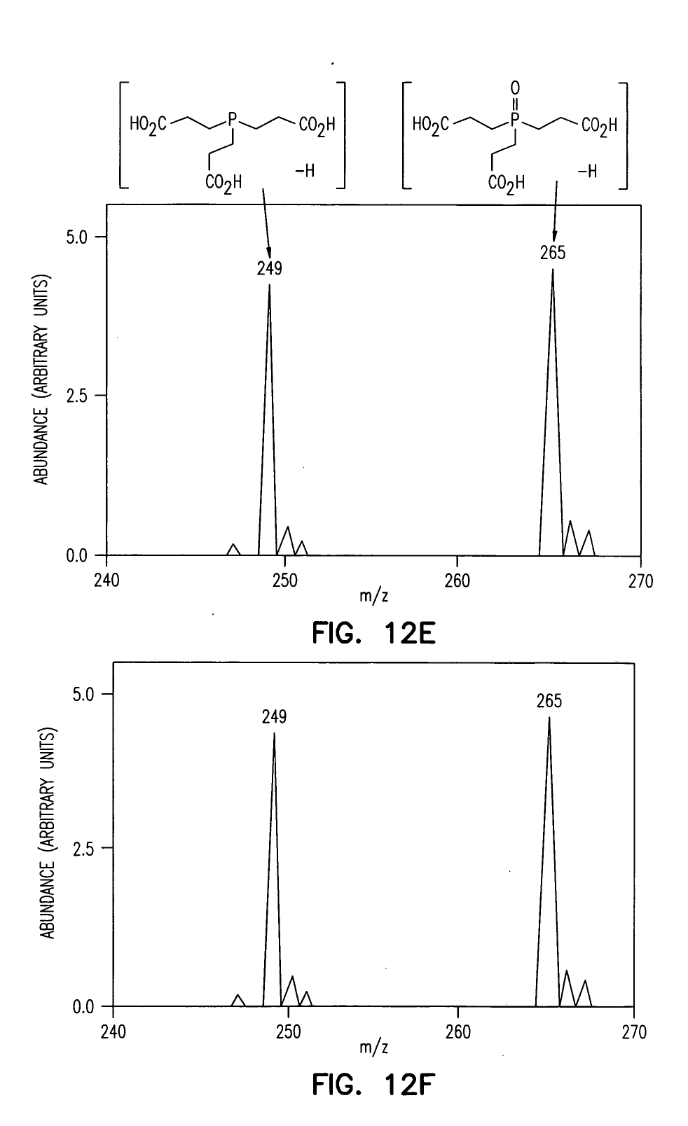

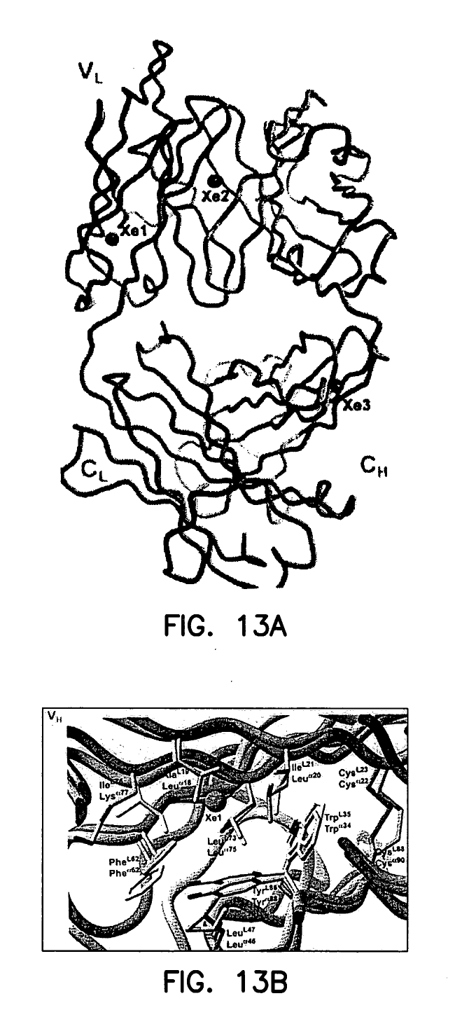

Oxygen isotope experiments were undertaken to test the hypothesis of an antibody-catalyzed photo-oxidation of H₂O by ¹O₂ through determination of the source of oxygen found in the H₂O₂. Contents of ¹⁶O/¹⁸O in H₂O₂ were measured by modification of a standard H₂O₂ detection method (Han et al., Anal. Biochem., 234, 107 (1996)). Briefly, this method involves reduction with tris carboxyethyl phosphine (TCEP), followed by mass-spectral (MS) analysis of the corresponding phosphine oxides (FIG. 12).

These experiments revealed that UV-irradiation of antibodies, in the presence of oxygen, leads to oxygen incorporation from water into H₂O₂ (FIGS. 12A and 12B). The relative abundance of the ¹⁶O/¹⁸O ratio observed in the MS of the phosphine oxide after irradiation of sheep poly-IgG under conditions of saturating ¹⁶O₂ concentration in a solution of H₂¹⁸O (98% 180) phosphate buffer (PB) is 2.2±0.2:1 (FIG. 12A). When the converse experiment is performed, with an ¹⁸O enriched molecular oxygen mixture (90% ¹⁸O) in H₂¹⁶O PB, the reverse ratio (1:2.0±0.2) is observed (FIG. 12B). These values of the ratios exhibit good reproducibility (+10%, n=10) and are found for all antibodies studied.

The following control experiments were performed. First, under conditions of ¹⁶O₂ and H₂¹⁶O, irradiation of poly-IgG (horse) generated H₂¹⁶O₂ (FIG. 12C). There is no incorporation of ¹⁸O when H₂¹⁶O₂ (400 μM in PB, pH 7.0) itself is irradiated for 4 hours in H₂¹⁸O. This result alleviates concerns that ¹⁸O incorporation into H₂O₂ may be occurring via either an acid-catalyzed exchange with water or by a mechanism that involves homolytic cleavage of H₂¹⁶O₂ and recombination with H¹⁸O^{?} from water. To check the possibility that antibodies may catalyze both the production of H₂¹⁶O₂ and its acid-catalyzed exchange with H₂¹⁸O, the isotopic exchange of H₂¹⁶O₂ (200 μM) in H₂¹⁶O₂ (98% ¹⁸O) PB in the presence of sheep poly-IgG (6.7 μM) after UV-irradiation under an inert atmosphere was determined. Only a trace of incorporation of ¹⁸O into H₂¹⁶O₂ was observed (FIG. 12D).

Isotope experiments were also performed with β-galactosidase, the most efficient non-immunoglobulin protein at generating H₂O₂, as well as 3-methylindole. In both cases, photo-oxidation led to negligible ¹⁸O incorporation into the H₂O₂ (FIGS. 12E and 12F), illustrating the view that the indole ring itself and tryptophan residues in this protein are behaving simply as reductants of ¹O₂.

This view is further supported because irradiation of 3-methylindole generates H₂O₂ that does not include oxygen incorporation from H₂¹⁸O. The same experiment performed with tryptophan does give rise to exchange with a ratio ¹⁶O/¹⁸O 1.2:1. This result is thought to be due to the ammonium functionality acting as an intramolecular general acid, protonating the internal oxygen of a diastereomeric mixture of 3′-hydroperoxides. It should be noted that while this is interesting from a chemical point of view, it cannot account for the catalytic production of H₂O₂ by antibodies both because it is a stoichiometric process and Trp residues in proteins do not possess a free ammonium group.

The chemical mechanism. All antibodies studied can catalyze the oxidation of water by singlet oxygen. The thermodynamic balance between reactants and products for the oxidation of H₂O by ¹O₂ (heat of reaction, ? Hr=+28.1 kcal/mol) (D. R. Lide, in Handbook of Chemistry and Physics, 73^{rd} ed. (CRC, 1992)), demands a stoichiometry in which more than one molecule of ¹O₂ would have to participate per molecule of oxidized water during its conversion into two molecules of H₂O₂. This stoichiometry assumes that no further light energy before that involved in the production of singlet from triplet oxygen is participating in the process. Qualitative chemical reasoning on hypothetical mechanistic pathways, together with thermodynamic considerations, makes the likely overall stoichiometries as in either equations lb or c (all energetics are calculated from gas phase experimental heats of formation and are reported in kcal/mol):

| | | |
|---|---|---|
| ¹O₂ + 2H₂O ? | 2H₂O₂; | ? Hᵣ^{o} = 28.1 (1a) |
| 2 ¹O₂ + 2H₂O ? | 2H₂O₂ + ³O₂; | ? Hᵣ^{o} = 5.6 (1b) |
| 3 ¹O₂ + 2H₂O ? | 2H₂O₂ + 2 ³O₂; | ? Hᵣ^{o} = −16.9 (1c) |

A recent report of a transition metal-catalyzed conversion of ¹O₂ and water into hydrogen peroxide, via a tellurium-mediated redox process (Detty and Gibson, J. Am. Chem. Soc., 112, 4086 (1990)), provides experimental evidence for a process in which ¹O₂ and H₂O can be converted into H₂O₂ and, hence that the energetic demands of this process can be overcome. It is thought that the mechanism for the antibody-mediated photo-oxidation process involves the addition of a molecule water to a molecule of ¹O₂ to form dihydrogen trioxide as the first intermediate on the way to H₂O₂. The antibody's function as a catalyst would have to be the supply of a specific molecular environment that would stabilize the critical intermediate relative to its reversible formation and, or, would accelerate the consumption of the intermediate by channeling its conversion to H₂O₂. An essential feature of such an environment might consist of a special constellation of organized water molecules at an active site conditioned by an antibody-specific surrounding.

While H₂O₃ has not yet been detected in biological systems, its chemistry in vivo has been a source of considerable speculation and its in vitro properties have been the subject of numerous experimental and theoretical treatments (C. Deby, La Recherche, 228, 378 (1991); Sawyer, in Oxygen Chemistry (Oxford University Press, Oxford, 1991); Cerkovnik and Plesnicar, J. Am. Chem. Soc., 115, 12169 (1993); Vincent and Hillier, J. Phys. Chem., 99, 3109 (1995); Plesnicar et al., Chem. Eur. J., 6, 809 (2000); Corey et al., J. Am. Chem. Soc., 108, 2472 (1986); Koller and Plesnicar, J. Am. Chem. Soc. 118, 2470 (1996); Cacace et al., Science, 285, 81 (1999)). Plesnicar and co-workers have shown that H₂O₃, reductively generated from ozone, decomposes into H₂O and ¹O₂ (Koller and Plesnicar, J. Am. Chem. Soc., 118, 2470 (1996)). Applying the principle of microscopic reversibility, it was surmised that the reverse reaction should also be catalyzed by one or more molecules of water. To delineate plausible reaction routes and energetics of such a process, first principles quantum chemical (QC) methods were used (B3LYP Density Functional Theory) as described herein. The results are illustrated in equations 2a-c (all energetics are in kcal/mol):

| H₂O + ¹O₂ | ? | TS | ? | H₂O₃ | (2a) |
|---|---|---|---|---|---|
| 0.0 | | 69.5 | | 15.5 | |
| 2H₂O + ¹O₂ | ? | TS | ? | H₂O₃ + H₂O | (2b) |
| 0.0 | | 31.5 | | 15.5 | |
| 3H₂O + ¹O₂ | ? | TS | ? | H₂O₃ + 2H₂O | (2c) |
| 0.0 | | 15.5 | | 15.5 | |

The direct reaction of water and ¹O₂ to give H₂O₃ is quite unfavorable, with an activation barrier of 70 kcal/mol (Eqn. 2a). However, with the addition of a second or third water molecule a concerted process is found that decreases the activation barrier to 31.5 and 15.5 kcal/mol respectively. Indeed these additional waters do play the role of a catalyst (in equation. 2b the H of the 2nd water goes to the product HOOOH, simultaneous with the H of the 1st water replacing it). These barriers are small compared with the first HO bond energy of water (119 kcal/mol) and the bond energy of ¹O₂ (96 kcal/mol). Note that the reverse reaction in eqn. 2b and eqn. 2c has a barrier of only 15.5 or 0 kcal/mol respectively, suggesting that H₂O₃ is not stable in bulk water or water rich systems. Thus, the best site within the antibody structure for producing and utilizing H₂O₃ is expected to be one in which there are localized waters and water dimers next to hydrophobic regions without such waters.

The ¹⁶O/¹⁸O ratio in the phosphine oxide derived from the antibody-catalyzed photo-oxidation of water poses a significant constraint to the selection of reaction paths by which this primary intermediate H₂O₃ would to convert to the final product H₂O₂. The ratio is primarily determined by the number of ¹O₂ molecules that chemically participate in the production of two moles of H₂O₂ from two moles of H₂O as well as by mechanistic details of this process. A ratio of 2.2:1 would coincide exactly with the value predicted for certain mechanisms in which two molecules of ¹O₂ and two molecules of H₂O are transformed into two molecules of H₂O₂ and one molecule of molecular oxygen (which would have to be ³O₂ for thermodynamic reasons). An example of such a mechanism is an SN2-type disproportionation of two molecules of H₂O₃ into H₂O₄ and H₂O₂, followed by the decomposition of the former into H₂O₂ and ³O₂. The complex problem of defining theoretically feasible reaction pathways for the conversion of H₂O₃ into H₂O₂ with or without the participation of ¹O₂ has been tackled in a systematic way using quantum chemical methods (B3LYP Density Functional Theory). These studies show extensive docking calculations of H₂O₃ and the transition states for its formation and conversion into H₂O₂ to a number of proteins. Indeed there are unique sites of stabilizing these species in a region of antibodies (and the aβ-T cell receptor) in a region with isolated waters and next to hydrophobic regions. This extended study revealed the potential existence of a whole spectrum of theoretically feasible chemical pathways for the H₂O₃ to H₂O₂ conversion.

Structural studies of xenon binding to antibodies. Given the conserved ability of antibodies, regardless of origin or antigen specificity, or of the aβ-TCR to mediate this reaction, X-ray structural studies were instigated to search for a possible conserved reaction site within these immunoglobulin fold proteins. A key constraint for any potential locus is that molecular oxygen (either ¹O₂ or triplet with a potential sensitizing residue in proximity, preferably tryptophan) and water must be able to co-localize, and the transition-states and intermediates along the pathway must be stabilized either within the site or in close proximity.

There is strong evidence to support the notion that Xe and O₂ co-localize in the same cavities within proteins (Tilson et al., J. Mol. Biol., 199, 195 (1988); Schoenborn et al., Nature, 207, 28 (1965)). Accordingly, xenon gas was used as a heavy atom tracer to locate cavities within the murine monoclonal antibody 4C6 that may be accessible to molecular oxygen (Li et al., J. Am. Chem. Soc., 117, 3308 (1995)).

Three xenon sites were identified (FIG. 13A), and all occupy hydrophobic cavities as observed in other Xe-binding sites in proteins (Scott and Gibson, Biochemistry, 36, 11909 (1997); Prangë et al., PROTEINS: Struct., Funct. and Genet., 30, 61 (1998)). Superposition of the refined native and Xe-derivatized structures shows that, aside from addition of xenon, there is little discernible change in the protein backbone or side chain conformation or in the location of bound water molecules.

The xenon I binding site (Xe1 site) has been analyzed here in more detail because it is conserved in all antibodies and the aB TCR (FIG. 13B). Xel is in the middle of a highly conserved region between the β-sheets of V_{L}, 7 ? from an invariant Trp. The Xe1 site is sandwiched between the two β-sheets that comprise the immunoglobulin fold of the _{V}L, approximately 5 ? from the outside molecular surface. Xenon site two (Xe2) sits at the base of the antigen binding pocket directly above several highly conserved residues that form the structurally conserved interface between the heavy and light chains of an antibody (FIG. 13A). The residues in the V_{L} V_{H} interface are primarily hydrophobic and include conserved aromatic side chains, such as Trp^{H109}.

The contacting side chains for Xe1 in Fab 4C6 are Ala^{L19}, Ile^{L21}, Leu^{L73}, and Ile^{L75}, which are highly conserved aliphatic side chains in all antibodies (Kabat et al.,

Sequences of Proteins of Immunological Interest (US Department of Health and Human Services, Public Health Service, NIH, ed. 5th, 1991)). Additionally, only slight structural variation was observed in this region in all antibodies surveyed. Notably, several other highly conserved and invariant residues are in the immediate vicinity of this xenon site, including Trp^{L35}, Phe^{L62}, Tyr^{L86}, Leu^{L104}, and the disulfide-bridge between Cys^{L23} and Cys^{L88}. Trp^{L35} stacks against the disulfide-bridge and is only 7 ? from the xenon atom. In this structural context, Trp^{L35} may be a putative molecular oxygen sensitizer, since it is the closest Trp to Xe1. Comparison with the 2C aβ TCR structure and all available TCR sequences shows that this Xe1 hydrophobic pocket is also highly conserved in TCRs (FIG. 5B) (Garcia, Science, 274, 209 (1996)).

Human β₂-microglobulin, which does not generate H₂O₂, does not have the same detailed structural characteristics that define the antibody Xe1 binding pocket, despite its overall immunoglobulin fold. Also, β₂-microglobulin does not contain the conserved Trp residue that occurs there in both antibodies and TCRs. If Trp^{L35} (antibodies) or Trp^{a34} (TCR) is the oxygen sensitizer, the lack of a corresponding Trp in β₂-microglobulin may relate to the finding that it does not catalyze the oxidation of water.

Thus, the xenon experiments have identified at least one site that is both accessible to molecular oxygen and is in a conserved region (V_{L}) in close proximity to an invariant Trp; an equivalent conserved site is also possible in the fold of V_{H}. The structure and sequence around the Xe1 site is almost exactly reproduced in the V_{H} domain by the pseudo two-fold rotation axis that relates V_{L} to V_{H}. Although a xenon binding-site was not located in this domain, it is thought that molecular oxygen can still access the corresponding cavity in V_{H}. The proposed heavy chain xenon site may not have been found because the crystals were pressurized for only two minutes, which may have been insufficient time to establish full equilibrium, or simply because xenon is too large compared to oxygen for the corresponding cavity on the V_{H} side, or due to crystal packing. In other antibody experiments, Xe binding sites were found in only one of the two molecules of the asymmetric unit that suggests that crystal packing can modulate access of Xe in crystals. Analysis of the sequence and structure around these sites shows that they are highly conserved in both antibodies and TCRs thus providing a possible understanding of why the Ig-fold in antibodies and the TCR can be involved in this unusual chemistry.

Antibodies are unique among proteins in their ability to catalytically convert ¹O₂ into H₂O₂. It is thought that this process participates in killing by event-related production of H₂O₂. Alternatively, antibodies can fulfill the function of defending an organism against ¹O₂. This would require the further processing of hydrogen peroxide into water and triplet oxygen by catalase.

### EXAMPLE III

### Antimicrobial Activity of Antibodies Materials and Methods

Antibody and Cell Preparations

Sheep (31243) and horse (31127) polyclonal IgG were obtained from Pierce and used without further purification. The E. coli O112a,c-specific murine monoclonal antibody (15404) was obtained from QED biosciences and was used without further purification. The E. coli non-specific murine monoclonal antibodies 33F12 and 84G3 were obtained from the Scripps Hybridoma lab and used at >98% purity (based on SDS-PAGE analysis). Monoclonal 33F12 is a murine monoclonal IgG that catalyzes the aldol reaction. Wagner et al., Science 270, 1797 (1995). E. coli XL1-B was obtained from Stratagene. E. coli O112a,c (ATCC 12804) is an enteroinvasive strain which can infect malnourished and immuno-compromised individuals. L. Siegfried, M. Kmetove, H. Puzova, M. Molokacova, J. Filka, J. Med. Microbiol. 41, 127 (1994).

The following antibody preparations were prepared in-house by the following methods.

Rabbit Polyclonal IgG Specific for E. coli XL-1 Blue.

On the day of immunization (Day 0), New Zealand White rabbits, (2.5 kg) were pre-bled 10 ml from each ear and then injected subcutaneously with heat killed (65° C., 15 min), chemically competent E. coli XL-1 (OD₆₀₀=1) (650 μl and 350 μl of phosphate buffered saline, PBS ph 7.4). Fourteen days after immunization (Day 14), the rabbits received a second injection in the same manner as the first. Twenty eight days after immunization (Day 28), the rabbits received a third injection in the same manner as the first and second injections. At thirty five days after immunization (Day 35), the rabbits were bled 50 ml from an ear. At forty two days after immunization, (Day 42), the rabbits were bleed 50 ml from an ear.

Sera were allowed to stand at room temperature for 1-2 h, then placed at 4° C. overnight and spun at 2500-3500 rpm for 15 min. The supernatants were transferred to a new round bottom tube (50 ml) and spun at 9-10 K rpm for 15 min. These supernatants were transferred to a clean conical (50 ml) tube and stored at −10° C. Sera were then tested by ELISA (see below), diluted 1:1 in PBS and then filtered through a 0.2 μM filter. The protein concentration (Abs₂₈₀) of sera samples was measured. Sera samples were then loaded onto a protein G column (Amersham Gamma-Bind G, 10 mg protein/ml bead). The bound antibody was washed with 3 column volumes of PBS pH 7.4 and then eluted with 2 column volumes of acetic acid (0.1 M, pH 3.0). The elution peak was neutralized with Tris buffer (1 M, pH 9.0) (0.5 ml in 4 ml fraction) and then dialyzed back into PBS.

Murine Monoclonal IgGs Specific for E. coli XL-1 Blue

At Day 0, 129 Gix+mice (6-8 weeks, 4 per group) received intraperitoneal injections of heat killed (65° C., 15 min), chemically competent E. coli XL-1 at OD₆₀₀=1 in a volume of 150 μl with 50 μl of phosphate buffered saline, PBS pH 7.4. At Day 14, the mice received a second injection in the same manner as the first. At Day 28, the mice received a third injection in the same manner as the first and second injections. At Day 35 mice were bled via intraocular puncture.

Twelve monoclonal antibodies specific for XL-1 blue were prepared using standard protocols. Antibody preparations were purified by ammonium sulfate precipitation followed by loading onto a protein G column (Amersham Gamma-Bind G, 10 mg protein/ml bead). The bound antibody was washed with 3 column volumes of PBS pH 7.4 and then eluted with two column volumes of acetic acid (0.1 M, pH 3.0). The elution peak was neutralized with Tris buffer (1 M, pH 9.0) (0.5 ml in 4 ml fraction) and then dialyzed back into PBS.

Generic ELISA for Determining Antibody-Binding to Live or Killed E. coli

The OD₆₀₀ of a frozen glycerol stock of E. coli XL1-blue was read and the live bacterial stock was diluted in PBS to OD₆₀₀=1.0. Twenty-five microliter aliquots of bacteria were placed in wells of a 96-well hi-bind ELISA plate and allowed to dry overnight at 37° C. Plates were gently washed twice with dH₂O. Plate wells were blocked with BLOTTO (50 μl/well) for 30 min at room temperature and this blocking solution was removed by shaking. The antibody-containing sample to be assayed was then diluted into BLOTTO and 25 μl of this solution was placed in each well. Plates were incubated at 37° C. for 1 h in a moist chamber, washed with dH₂O (10×) and 25 μl of a secondary antibody (HRP-goat anti-rabbit conjugate, 1:2000) in BLOTTO was added to each well. Plates were incubated at 37° C. for 1 h in a moist chamber and washed gently with dH₂O (10×). Developer substrate (50 μl/well) was added and the plates were read at 450 nm after 30 min.

Dead bacterial samples were also used for ELISA. These samples were handled in the same manner as above, but before addition and adherence to ELISA microtiter plates, the E. coli are heat killed (65° C., 15 min).

Bactericidal Assays

In a typical experiment, a culture of E. coli (in log phase growth, OD₆₀₀=0.2-0.3) was repeatedly pelleted (3×3,500 rpm) and resuspended in PBS (pH 7.4). The PBS suspended cells were then added to glass vials and cooled to 4° C. Hematoporphyrin IX (40 μM) and antibody (20 μM) were added and the vials were either placed on a light box (visible light, 2.8 mW cm⁻²) or in the dark at 4° C. and incubated for 1 h. Viability was determined by recovery of colony forming units (CFUs) on agar plates. Each experiment was performed at least in duplicate.

Microscopy Studies

Samples were prepared for electron microscopy as follows. Cells were fixed with paraformaldehyde (2% w/v), glutaraldehyde (2.5% w/v) in cacodylate (0.1 M) at 0° C. for 1.75 h and then pelleted. The cell pellet was resuspended in OSO₄ (1% W/V) in cacodylate (0.1 M), allowed to stand for 30 min and then pelleted. The pellet was then sequentially dehydrated with ethanol and propylene oxide, embedded in resin and then sectioned. The sections were stained with uranyl acetate and lead citrate. For gold labeling studies, the procedure used was as detailed above with the addition of the following steps. First, samples were pelleted and washed with fresh isotonic buffer to remove unbound primary antibody. Second, the pellet was resuspended in a solution of goat anti-mouse antibody that had been covalently modified with 12 nm gold particles, and incubated for 90 min.

Decomposition of O₃ Under Aqueous Conditions

The rate of decomposition of O₃ under the aqueous conditions employed was measured by the following method. Ozone, produced by a passage of O₂ through a Polymetrics ozonizer, was bubbled for 2 min through a phosphate buffered saline (PBS, pH 7.4) solution in a quartz cuvette (1 cm²) at room temperature. The time-dependent change in optical density was then measured at 260 nm (ε=2,700 M⁻¹ cm⁻¹) for at least 5 half lives in a Hitachi u.v./vis spectrophotometer equipped with a thermostatted rack at 22° C. See Takeuchi et al., Anal. Chim. Acta. 230, 183 (1990). The half-life of O₃ was then determined graphically (t1/2=66 sec) from a plot of OD vs. time using Graphpad Prism V 3.0 software (data not shown). The sensitivity of the assay was limited by spectrophotometer accuracy to ±0.1% (˜1 μM) of the OD at t=0.

Assay for Ozone

In a typical experiment, a solution of indigo carmine 1 (1 mM) in PBS (pH 7.4) was irradiated on a transilluminator (312 nm, 0.8 mWcm⁻²) at room temperature in the presence or absence of antibody (20 μM) with or without catalase (13 mU/mL) in a quartz microtiter plate (final volume 200 μL), in duplicate. At various time-points a sample is removed (20 μL) and quenched into phosphate buffer (100 mM, pH 3.0, 180 μL). The OD was measured at 610 nm in a microtiter plate reader (Spectramax). Production of isatin sulfonic acid 2 was determined by LC-MS (Hitachi D-7000 HPLC linked to a Hitachi M-8000 ion-trap electrospray mass-spectrometer (in the negative-ion detection mode). LC conditions were a Spherisorb RP-C18 column and acetonitrile water (30:70) mobile phase at 1 mL/min. An in-line splitter was used to divert 0.2 mL/min of column effluent into the MS. Isatin sulfonic acid 2 RT=3.4 min, [MH]-226.

A variety of reactive species were tested to ascertain whether indigo carmine 1 could be converted to isatin sulfonic acid 2 by species other than ozone.

TABLE 2
Observed oxidation of indigo carmine 1^{a} and
¹⁸O isotope incorporation into cyclic
α-ketoamide 2^{b} by different reactive
oxygen species.

| oxidant | Reaction to form 2 | ¹⁸O incorporation into 2 |
|---|---|---|
| O₃^{c} | yes | Yes |
| ¹O₂^{*d} | yes | No |
| H₂O₃^{e} | yes | No |
| HO₂•/O₂•^{-f} | no | —^{h} |
| H₂O₂^{g} | no | —^{h} |
| HOClⁱ | no | —^{h} |

^{a}Oxidation was determined by following the absorbance change at 610 nm in a microtitre plate reader before and after addition of the respective oxidant to indigo carmine 1 (1 mM) in phosphate buffer (PB, pH 7.4) at room temperature under the conditions specified.

^{b18}O incorporation was determined by performing the oxidation of indigo carmine 1 in PB (100 mM, pH 7.4) with H₂ ¹⁸O (>95% labeled) under the conditions specified for each oxidant and monitoring the isotopic profile of cyclic α-ketoamide 2 by negative ion electrospray mass spectrometry. Under the conditions of the assay the label installed into the amide carbonyl of α-ketoamide 2 does not exchange with water.

^{c}Indigo carmine (1, 1 mM) was added to a solution of ozone (˜600 μM) in PB (100 mM, pH 7.0).

^{d}The effect of ¹O₂* was investigated by irradiation of an hematoporphyrin IX (40 μM) solution and 1 (1 mM) in PB with visible light (2.7 mW/cm⁻²) for 1 h.

^{e}See ref. 42.

^{f}potassium superoxide (10 mM) in DMSO was added to a solution of 1 in PB (100 mM, pH 7.0) such that the final organic cosolvent was 5%.

^{g}Final concentration 2 mM in PB.

^{h}Not determined.

ⁱIndigo carmine (1, 1 mM) was added to a solution of NaOCl (20 mM) in PBS (pH 7.4) and formation of cyclic α-ketoamide 2 was determined by HPLC after complete bleaching of the solution occurred.

Preliminary studies revealed that, rapid and reversible exchange of the oxygen of the lactam carbonyl of cyclic α-ketoamide 2 with water occurred in the presence of u.v. light (312 nm, 0.8 mW cm⁻²). However, in white light no discernable exchange occurred during the experiment. Thus, all ¹⁸O isotope incorporations experiments were carried out using hematoporphyrin IX (40 μM) and white light (2.7 mW cm ²) as the ¹O₂* source.

Further studies were performed using the following additional chemical probes that contained a normal carbon-carbon double bond.

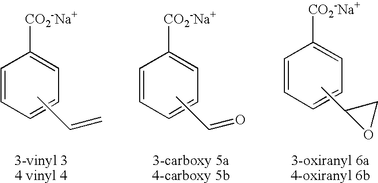

The choice of the probes, 3- and 4-vinyl-benzoic acid (3 and 4 respectively), was guided by their aqueous solubility coupled with ease of detection by HPLC. In a typical experiment, a solution of 3-vinyl benzoic acid 3 (1 mM) or 4-vinyl benzoic acid 4 (1 mM) in PBS (pH 7.4) was irradiated (312 nm, 0.8 mW/cm⁻²) at room temperature in the presence or absence of antibody 4C6, or sheep polyclonal antibody (20 μM). Timed aliquots were removed (20 μL) and diluted 1:3 into acetonitrile:water (1:1). Product composition was determined by reversed-phase HPLC.

Conventional ozonolysis of 3-vinyl benzoic acid 3 (1 mM) in PBS (pH 7.4) at room temperature leads to the production of the benzaldehyde derivative 5a with minor production of the corresponding epoxide 6a in a ratio of ˜10:1. Similarly, ozonolysis of 4, under the same conditions as described above, leads to 4-carboxybenzaldehyde 5b and the corresponding oxirane 5b in a ratio of 9:1. In a typical experiment, a solution of 3 or 4 (1 mM) in PBS (pH 7.4) was added to a solution of O₃ in PBS (600 μM) at room temperature and allowed to stand for 5-10 min. The ozonolysis of 3 and 4 was performed in this manner rather than by bubbling an O₃/O₂ mixture through the aqueous reaction solution to prevent further oxidation of 3 and 4 that leads to hydroxylation and fragmentation of the aromatic ring. The product mixture and substrate conversion was elucidated by reversed-phase HPLC. HPLC analysis was performed on a Hitachi D-7000 machine with a Spherisorb RP-18 column and a mobile phase of acetonitrile and water (0.1% TFA)(30:70) at a flow rate of 1 mL/min. Localization was performed by u.v. detection (254 nm) (RT 3=7.84 min; RT 5a=4.02 min; RT 6a=3.82 min; RT 4 8.50 min; RT 5b=3.72 min; RT 6b=4.25 min). Peak areas were converted to concentration by comparison to standard curves.

Antibody Detection on Neutrophils

Neutrophils are known to have antibodies on their cell surface. Fluorescence activated cell sorting (FACS) was used to measure the number of immunoglobulin molecules per cell present under resting and activated conditions. Under resting conditions there are approximately 50,000 antibody molecules per cell, which increased to approximately 65,000 antibody molecules per cell upon activation.

### Results

Antimicrobial Activity of Antibodies

As illustrated above, antibodies catalyze the generation of hydrogen peroxide (H₂O₂) from singlet molecular oxygen (¹O₂*) and water by a process that proceeds via dihydrogen trioxide (H₂O₃) intermediate. Results provided in this Example illustrate that antibodies can utilize this process to efficiently kill bacteria.

Initial bactericidal studies utilized two strains of the gram-negative bacteria E. coli (XL1-blue and O-112a,c). E. coli XL1-B was obtained from Stratagene. E. coli O112a,c (ATCC 12804) is an enteroinvasive strain which can infect malnourished and immuno-compromised individuals. Siegfried et al., J. Med. Microbiol. 41, 127 (1994).

The ¹O₂* ion has bactericidal action. Berthiaume et al., Biotechnology 12, 703 (1994). However, initiation of H₂O₂ production by antibodies requires exposure to the substrate ¹O₂*. Wentworth et al., Proc. Natl. Acad. Sci. U.S.A. 97, 10930 (2000). Therefore, a ¹O₂* generating system was used that would not, on its own, kill E. coli. Antibodies can utilize ¹O₂* generated by either endogenous or exogenous sensitizers or chemical sources, using u.v. or white light, or thermal decomposition of e.g. anthracene-9,10-dipropionic acid endoperoxide respectively. Therefore, the choice of a ¹O₂* generating system is guided solely by experimental considerations such as reaction efficiency and cellular or substrate sensitivity to irradiation. In these experiments, hematoporphyrin IX (HPIX, 40 μM) was selected as an efficient sensitizer of ³O₂. Wilkinson et al., J. Phys. Chem. Ref. Data 22, 113 (1993). When irradiated with white light (light flux 2.7 mW cm⁻²) for 1 h in phosphate buffered saline (PBS, pH 7.4) at 4±1° C., hematoporphyrin IX has negligible bactericidal activity against the two E. coli serotypes (˜107 cells/mL).

In a typical experiment, a culture of E. coli (in log phase growth, OD600=0.2-0.3) was repeatedly washed in PBS by pelleting (3×3,500 rpm) the cells and resuspending them in PBS (pH 7.4). The PBS suspended cells were then added to glass vials and cooled to 4° C. Hematoporphyrin IX (40 μM) and antibody (20 μM) were added and the vials were either placed on a light box (visible light, 2.8 mW cm⁻²) or in the dark at 4° C. and incubated for 1 h. Viability was determined by recovery of colony forming units (CFUs) on agar plates. Each experiment was performed at least in duplicate.

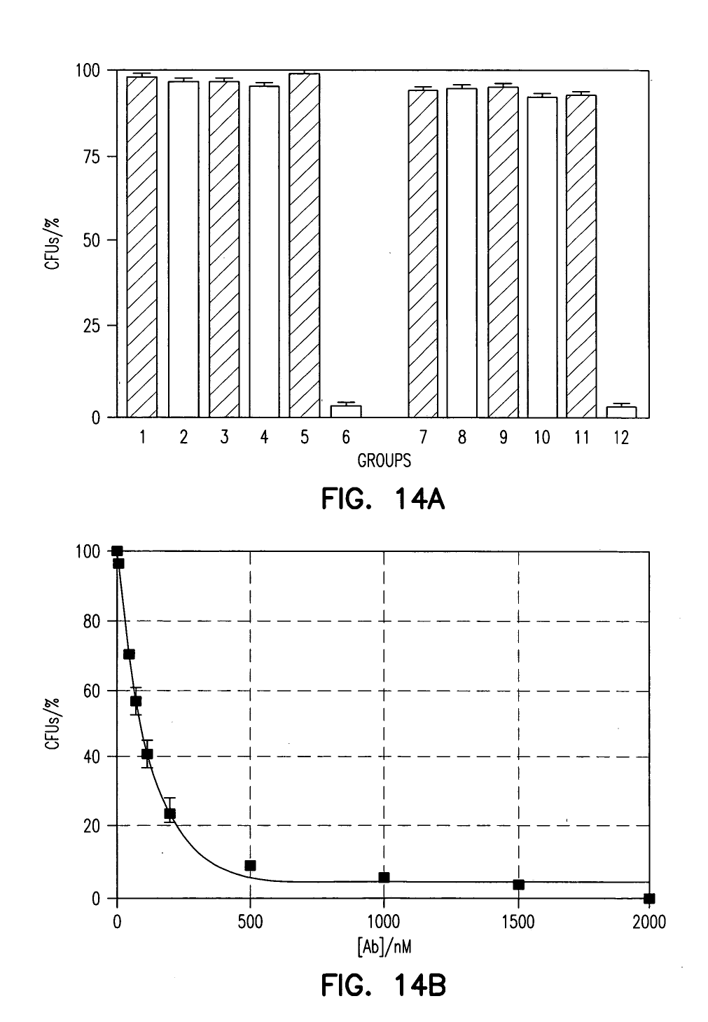

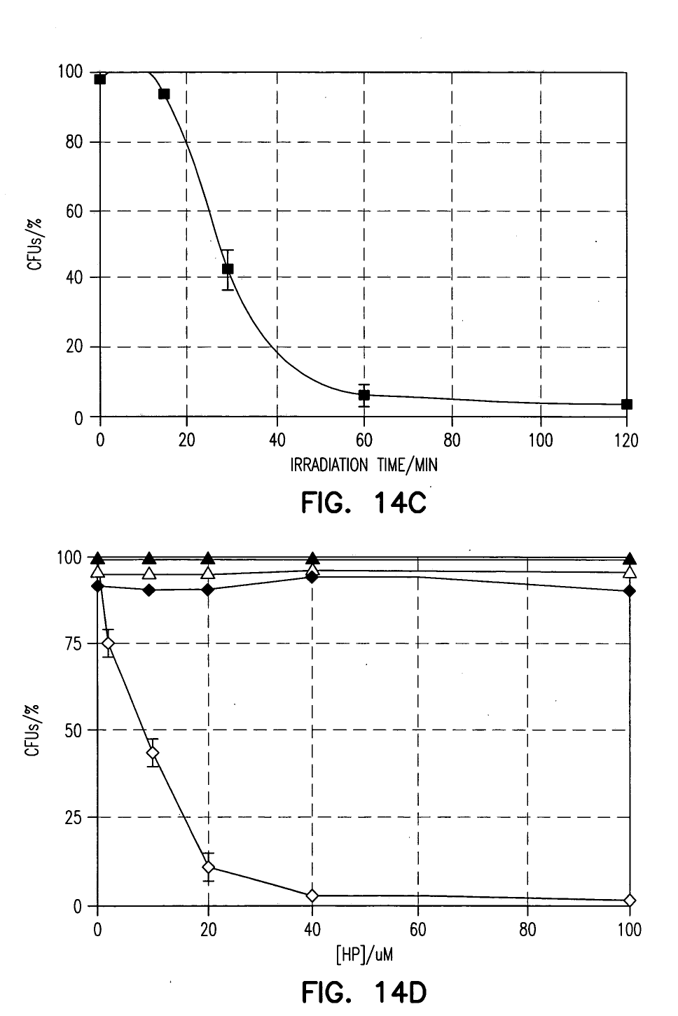

Addition of monoclonal antibodies (20 μM) to a mixture of hematoporphyrin IX and bacteria resulted in killing of >95% of the bacteria (FIG. 14A). The bactericidal activity of antibodies was a function of antibody concentration. For example, killing of >95% of O112a,c cells was achieved with 10 μM of the antigen-specific murine monoclonal antibody 15404. These data indicate that the effective antibody concentration that kills 50% of the cells (EC₅₀) was 81±6 nM (FIG. 14B). A similar concentration vs. kill dependence was observed for a specific monoclonal antibody (25D11) against the XL1-blue E. coli strain, with maximum killing >95% being observed at about 10 μM.

Antibody-mediated bactericidal activity increased both as a function of irradiation time (FIG. 14C) and with increasing hematoporphyrin IX concentration (the light flux was fixed at 2.7 mW cm-2) (FIG. 14D). The observation that antibody-mediated bacterial killing is proportional to both hematoporphyrin IX concentration and light irradiation indicated that both ¹O₂* and the water oxidation pathway have a key role in the process. Critically, in the absence of ¹O₂*, immunoglobulins have a negligible effect on the survival of E. coli.

Controls indicated that cold shock and hematoporphyrin IX toxicity were not responsible for an appreciable loss of colony forming units (CFUs). Furthermore, confocal microscopy revealed that antibody mediated bacterial cell aggregation was also not contributing to a lack of CFUs in the antibody-treated groups. Fluorescence analysis of the bacterial cells indicated that the amount of membrane-associated sensitizer in the hematoporphyrin IX-treated E. coli cells was not increased by antibody binding. Finally, while it is difficult to rule out the potential role of trace metals in the bactericidal action of antibodies, the presence of EDTA (2 mM) had no effect on the survival of bacteria in the assay system employed.

The bactericidal potential of antibodies appeared to be in general phenomenon. All twelve murine monoclonal antibodies (1×κγ, 7×κγ2a, 3×κγ2b, 1×κγ3 isotypes) and one rabbit polyclonal IgG (titer 120,000) sample that were tested were bactericidal. Nonspecific antibodies also were able to generate bactericidal agents. Only ¹O₂* was required for the activation of the water oxidation pathway—such activation was independent of the antibody-antigen union. In this regard, 10 non-specific murine monoclonal antibodies, one non-specific sheep antibody preparation and one horse polyclonal IgG sample with no specificity for E. coli cell-surface antigens were studied and all possessed bactericidal activity. The potency of the bactericidal activity of antigen non-specific antibodies was observed to be very similar to antigen-specific antibodies. Typically 20 μM of antibody (non-specific) was >95% bactericidal in the assay system. The bactericidal action of antibodies was not simply a non-specific protein effect as bovine serum albumin (BSA, 20 μM) exhibited no bacterial killing in the assay system.

To gain insight into the nature of the observed bacterial killing the morphology of killed bacteria was studied by electron microscopy. Gold-labeled secondary antibodies were used to correlate the morphological damage to sites on the bacterial cell wall where antibodies were bound.

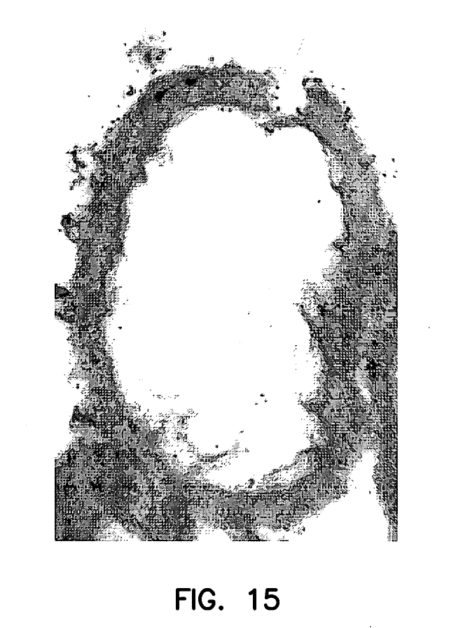

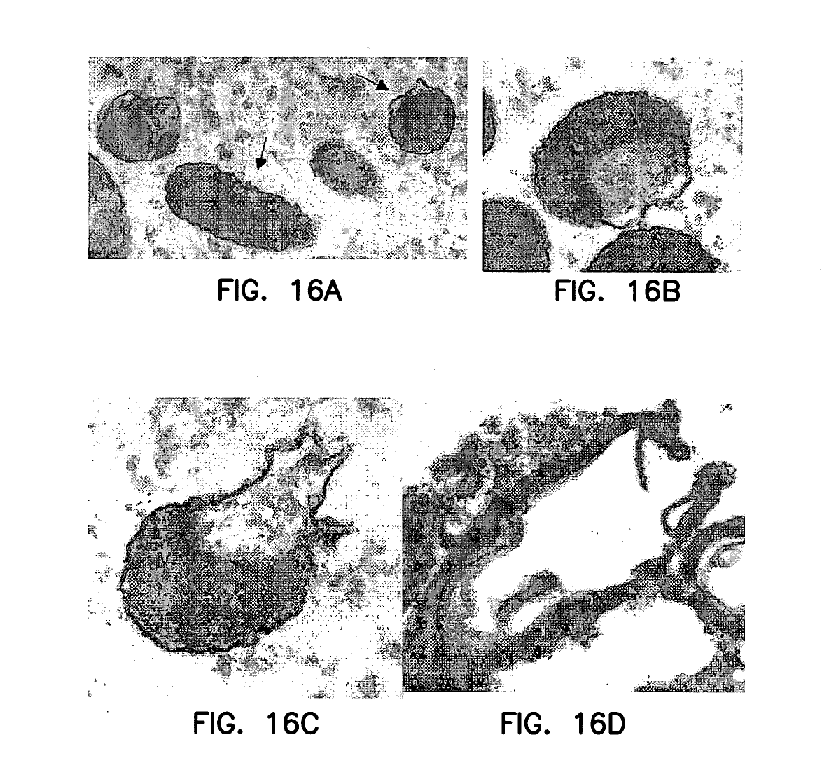

The killing is associated with the production of holes in the bacterial cell wall at the sites of antigen-antibody union (FIG. 15). The process appeared to be a gradual one as evidenced by the range of morphologies present within the bacteria sampled. There were clear stages in the bactericidal pathway, in which oxidative damage led to an increased permeability of the cell wall and plasma membrane to water.

The bacterium is under an internal pressure of about 30 atmospheres, hence any weakening of the membrane can lead to catastrophic rupture. The process appeared to begin with slight disruptions observed at the interface between the cell wall and cytoplasm (FIG. 16A) that became more severe with clear separation of the cell wall from the cytoplasmic contents (FIG. 16B). Continued influx of water resulted in gross distortion and deformity of the bacterial cell structure (FIG. 16C), ultimately leading to rupturing of the cell wall and plasma membrane and extrusion of the cytoplasmic contents at the sites of antibody attachment (FIG. 16D). In this regard, it is interesting that the observed morphologies induced by antibody-mediated killing are similar to those seen when bacteria are destroyed by phagocytosis. Hofinan et al., Infect. Immun. 68, 449 (2000).

The Chemical Nature of the Bactericidal Agents(s)

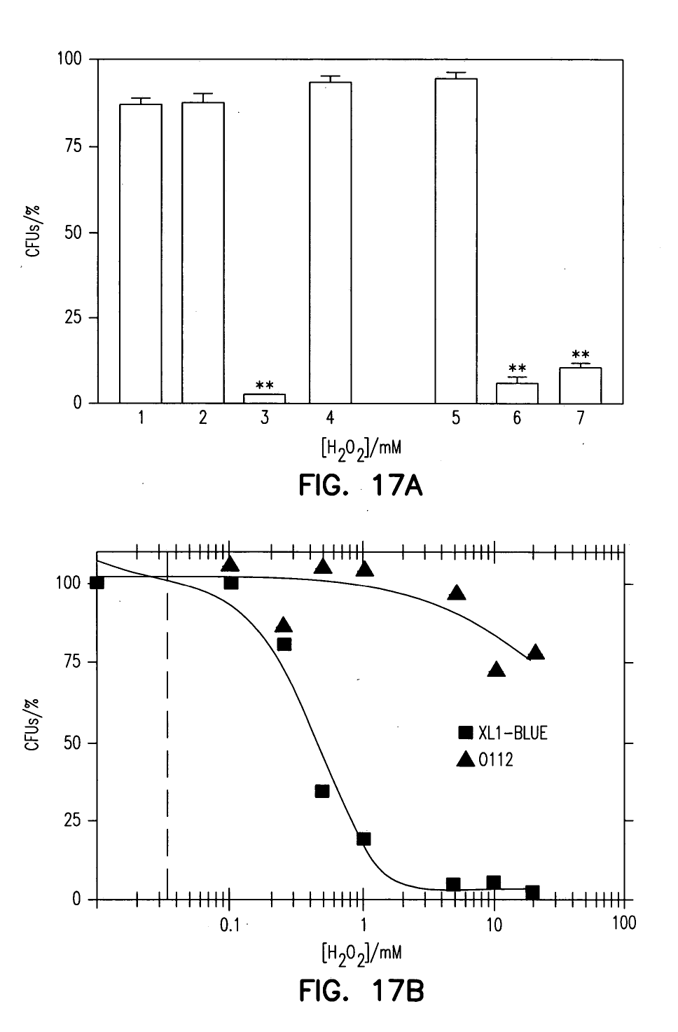

If H₂O₂ was the ultimate product of the antibody-catalyzed oxidation of water pathway (Wentworth et al., Proc. Natl. Acad. Sci. U.S.A. 97, 10930 (2000); P. Wentworth, Jr. et al Science 293, 1806 (2001)), then H₂O₂ alone would be the killing agent. This conclusion was strengthened by observations that catalase, which converts H₂O₂ to water (H₂O) and molecular oxygen (O₂), offered complete protection against the bactericidal activity of non-specific antibodies (FIG. 17A).

The amount of H₂O₂ generated by non-specific antibodies was 35±5 μM. The amount of H₂O₂ generated by specific antibodies was variable. The issue of proximity made a direct comparison between the effects of H₂O₂ in solution and H₂O₂ generated on the surface of the bacterial membrane complicated. For example, the protective effect of catalase (13 mU/mL) against the bactericidal activity of 111 E. coli antigen-specific murine monoclonal antibodies and 11 E. coli non-specific murine monoclonal antibodies was studied. In all cases with non-specific antibodies, catalase completely attenuated the bactericidal activity. For the antigen-specific antibodies however, extent of protection by catalase was dependent on the monoclonal antibody used and varied over a wide range. Therefore, proximity of H₂O₂ generation (directly on the surface of the bacterial membrane or in solution) affected the degree of protection offered by catalase. Hence, the effects of H₂O₂ in solution were compared only with H₂O₂ generated by antigen non-specific antibodies.

The mean rate of H₂O₂ formation (35±5 μM/h) generated by non-specific antibodies (20 μM) during the irradiation of a mixture containing hematoporphyrin IX (40 μM) with visible light (2.7 mW cm⁻²) for 1 h at 4° C. in PBS (pH 7.4) was highly conserved. This mean value was determined from ten murine monoclonal IgGs and a sheep and horse polyclonal IgG (n=12).

However, when the toxicity of H₂O₂ on the two E. coli cell lines was quantified it became apparent that the amount of H₂O₂ generated by non-specific antibodies, 35±5 μM, could not alone account for the potency of the bactericidal activity (FIG. 17B). This value was between 1 and 4 orders of magnitude below that required to kill 50% of the bacteria, depending on whether the cell-line is XL1-blue or O112a,c respectively.

The combination of H₂O₂ with antibodies and/or H₂O₂ with hematoporphyrin IX was not more toxic to bacteria than H₂O₂ alone. These variables were tested to ascertain whether some interaction might occur between H₂O₂ and other components in the assay that would account for the potency of the bactericidal activity. In particular, the following combination of conditions were tested for bactericidal activity against E. coli O112a,c:

1. H₂O₂ (2 mM) and non-specific antibody (20 μM);

2. H₂O₂ (2 mM) and antigen-specific antibody (20 μM); and

3. H₂O₂ (2 mM) and HPIX (40 μM).

Each group was irradiated for 1 h with visible light (2.7 mW cm⁻²) at 4° C. No enhancement in killing was observed for any of these combinations compared to that of H₂O₂ (2 mM) alone.

The finding that the toxicity of H₂O₂ to E. coli was below that generated by antibodies, necessitated re-examination of the experiments with catalase. One possibility was that H₂O₂ reacted with some other chemical species that was also generated by the antibody, to produce other bactericidal molecule(s) and thus, by destroying H₂O₂, catalase prevented formation of that other chemical species. Another alternative was that the bactericidal species that were formed on the way to H₂O₂ was also a substrate for catalase.

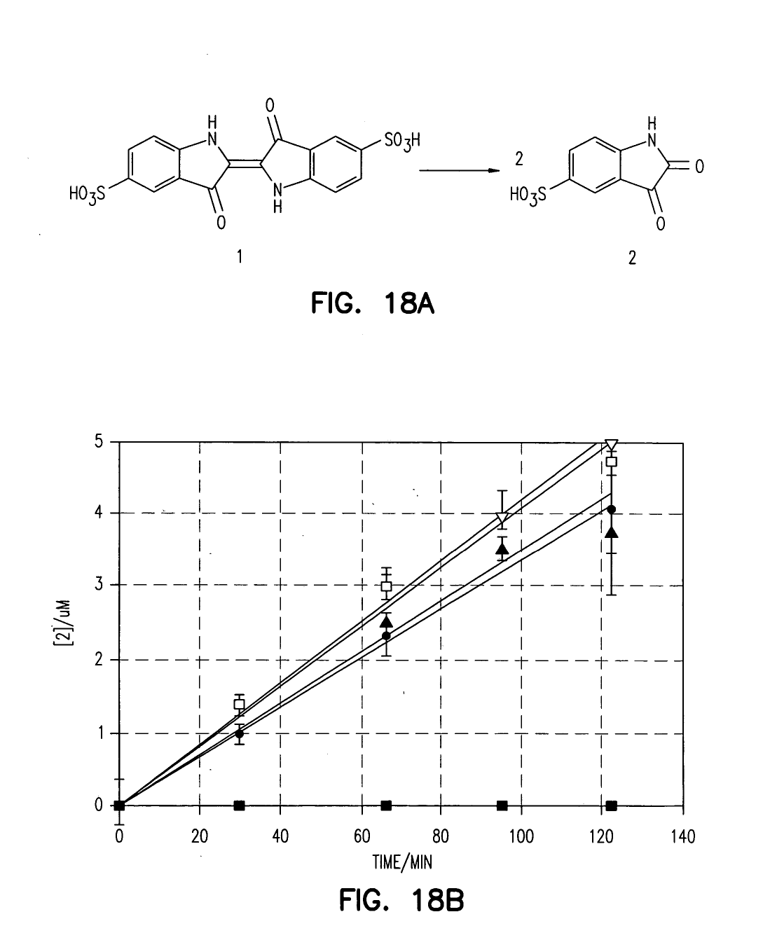

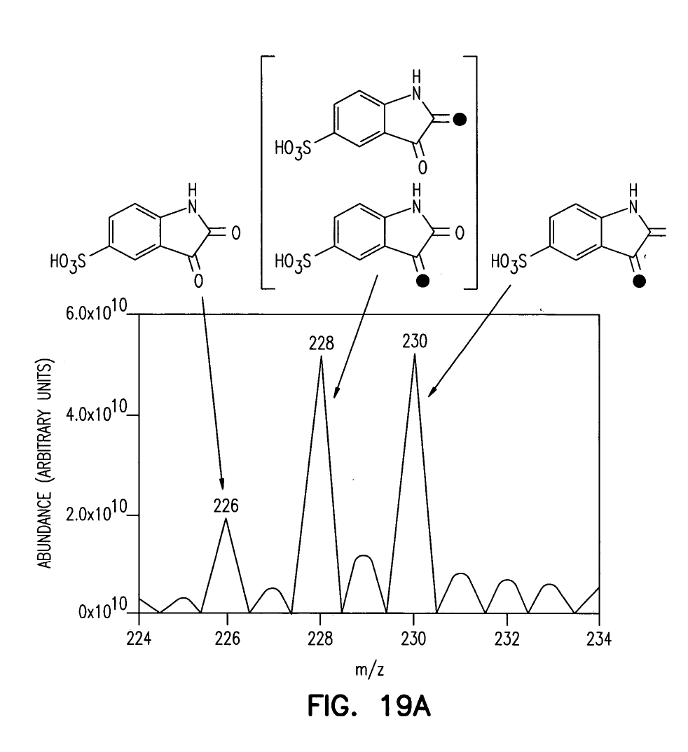

Further experimentation indicated that ozone (O₃) was generated by antibodies. Under the aqueous conditions employed, ozone is quite long lived (t1/2=66 sec). Thus, ozone is sufficiently long lived to be detected by chemical probes such as indigo carmine 1, a sensitive reagent for the detection of O₃ in aqueous systems. Takeuchi et al., Anal Chem. 61, 619 (1989); Takeuchi et al., Anal. Chim. Acta. 230, 183 (1990). Conventional ozonolysis of indigo carmine 1 in aqueous solution led to bleaching of the characteristic absorbance of indigo carmine 1 (γₘₐₓ 610 nm, ε=20,000 LM⁻¹cm⁻¹) and the formation of the cyclic α-ketoamide 2 (FIG. 18A).

To prove that ozone is produced by antibodies, the following experiments were performed. A solution of indigo carmine 1 (1 mM) in PBS (pH 7.4) was irradiated with u.v. light (312 nm, 0.8 mW cm⁻²) with no antibodies present. No bleaching was observed. However, when the same experiment was carried out in the presence of either a sheep polyclonal antibody (20 μM) or the murine monoclonal antibody 33F12 (20 μM) bleaching of indigo carmine 1 was observed (FIG. 18B). Electrospray mass-spectrometry and HPLC analyses confirmed that cyclic α-ketoamide 2 was formed in this process. Sheep polyclonal antibody and monoclonal antibody 33F12 yield 4.1 μM and 4.9 μM of cyclic α-ketoamide 2 after 2 h of irradiation (312 nm, 0.8 mW cm²) of indigo carmine 1 (1 mM), respectively. The initial rate of antibody mediated conversion of indigo carmine 1 into cyclic α-ketoamide 2 is linear, independent of the antibody preparation (sheep polyclonal IgG=34.8±1.8 nM min⁻¹, 33F12=40.5±1.5 nM min⁻¹) (FIG. 18B).

The oxidative cleavage of the C═C double bond of indigo carmine 1 is a sensitive probe for ozone detection. Takeuchi et al., Anal. Chem. 61, 619 (1989); Takeuchi et al., Anal. Chim. Acta. 230, 183 (1990). However, such cleavage was not specific for ozone. Further experiments with the oxidants listed in Table 2 were performed under the specified conditions to test whether those oxidants could also oxidize indigo carmine 1. Such experimentation confirmed that singlet oxygen (¹O₂) could bleach solutions of indigo carmine 1 to form cyclic α-ketoamide 2 by oxidative double bond cleavage. ¹O₂* is generated by antibodies upon u.v.-irradiation. Wentworth et al., Proc. Natl. Acad. Sci. U.S.A. 97, 10930 (2000); Wentworth et al., Science 293, 1806 (2001). An analytical differentiation between oxidative cleavage of indigo carmine 1 to cyclic α-ketoamide 2 by ¹O₂* versus one by O₃ was therefore sought.

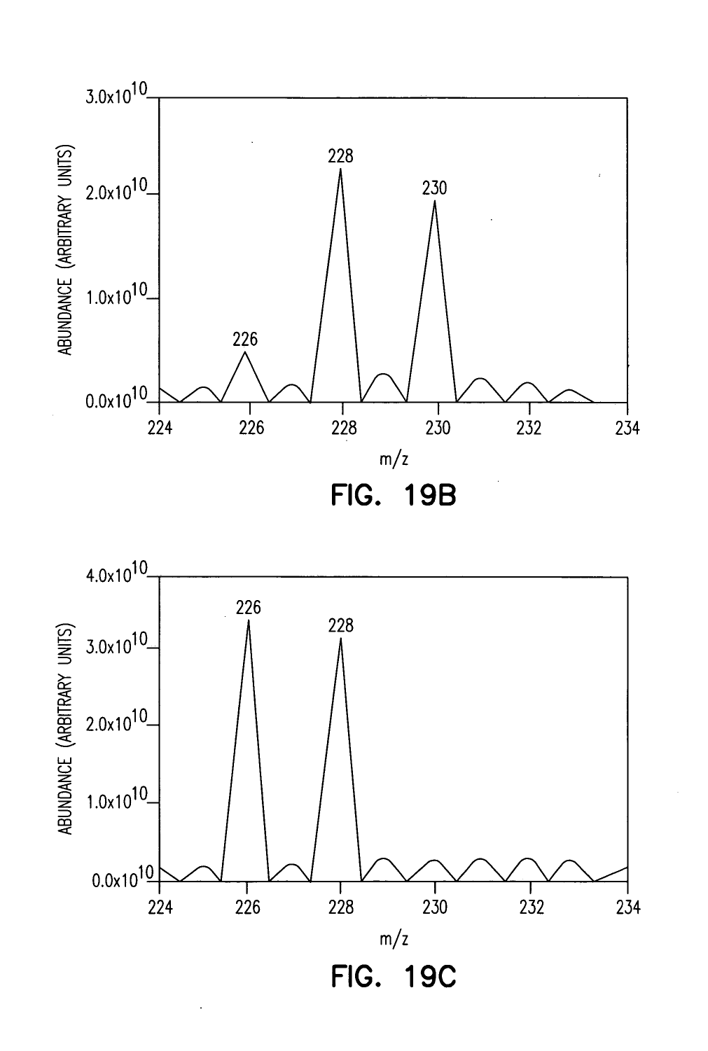

Further experimentation indicated that cleavage by O³ could be distinguished from cleavage by ¹O₂* by observing ¹⁸O incorporation into the lactam carbonyl groups of cyclic α-ketoamide 2 when ozone is the oxidant. No such ¹⁸O incorporation into the lactam carbonyl group of cyclic α-ketoamide 2 occurred when ¹O₂* was the oxidant. Isotope incorporation experiments were therefore carried out in H₂¹⁸0 (>95% 180) containing phosphate buffer (PB, 100 mM, pH 7.4) (Table 2 and FIG. 19), with the ¹O₂* being generated by irradiation of hematoporphyrin IX (40 μM) with visible light (2.7 mW cm⁻²). Preliminary experiments established that in ¹⁸O-water both indigo carmine 1 and 2 undergo slow but spontaneous isotope incorporation into the ketone-carbonyl groups of indigo carmine 1 as well as of 2, but not into the lactam carbonyl group of 2. Thus, the diagnostic marker in the mass spectrum of 2 was the [M−H]-230 fragment resulting from double isotope incorporation corresponding to ¹⁸O incorporation into both the ketone and lactam carbonyl groups of 2. Hence, in the mass spectrum of the oxidation product, the mass peak [M−H]-230 was observed when the oxidation of indigo carmine 1 was carried out in H₂¹⁸O by chemical ozonolysis (FIG. 19B), but not when indigo carmine 1 was oxidized by ¹O₂* (FIG. 19C). See Gorman et al., in Singlet Oxygen Chemistry, 205 (1988).

When indigo carmine 1 (100 μM) was irradiated with visible light (2.8 mW cm⁻²) in the presence of sheep IgG (20 μM) and hematoporphyrin IX (40 μM), oxidized product 2 was formed that possesses a mass spectrum demonstrating exchange of ¹⁸O of water into the lactam carbonyl (FIG. 19A). These data indicate that ozone was an oxidant for indigo carmine 1 when antibodies were present.

To further substantiate that ozone was generated by antibodies, the following additional chemical probes that contained a normal carbon-carbon double bond were tested.

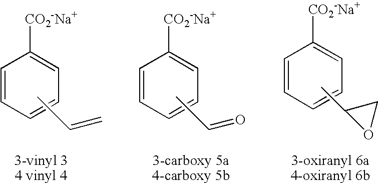

In a typical experiment, a solution of 3-vinyl benzoic acid 3 (1 mM) or 4-vinyl benzoic acid 4 (1 mM) in PBS (pH 7.4) was irradiated (312 nm, 0.8 mW/cm⁻²) at room temperature in the presence or absence of antibody 4C6, or sheep polyclonal antibody (20 μM). Timed aliquots were removed (20 μL) and diluted 1:3 into acetonitrile:water (1:1). Product composition was determined by reversed-phase HPLC.

Irradiation of solutions of compounds 3 and 4 (1 mM) with u.v. light (312 nm, 0.8 mW cm⁻²), in the presence of a sheep polyclonal IgG (20 μM), led to the formation of 3-carboxybenzaldehyde 5a and 3-oxiranyl benzoic acid 6a (ratio 15:1, 1.5% conversion of 3 after 3 h) and 4-carboxybenzaldehyde 5b and 4-oxiranyl-benzoic acid 6b (ratio of 10:1, 2% conversion to 4 after 3 h) respectively. These products are also observed when compounds 3 and 4 are ozonolyzed in a conventional way. Moreover, these results were similar to those observed for indigo carmine 1 irradiated with u.v. light in the presence of either a sheep polyclonal antibody or the murine monoclonal antibody 33F 12 where bleaching of indigo carmine 1 was observed (FIG. 18B). Again, if no antibodies were present, no bleaching was observed but in the presence of antibodies, oxidation products indicative of ozone were observed.

In sharp contrast, ¹O₂* generated by hematoporphyrin IX (40 μM) and visible light (2.7 mW cm⁻²), did not cause any detectable oxidation of either 3 or 4 under similar conditions. Therefore, 3-vinyl benzoic acid 3 and 4-vinyl benzoic acid 4 are selective for ozone and the ozone must be produced by the antibodies present in the reaction.

Evidence for Ozone Production by Activated Neutrophils

Neutrophils are central to a host's defense against bacteria and are known to have antibodies on their cell surface and the ability, upon activation, to generate a cocktail of powerful oxidants including ¹O₂*. Steinbeck et al., J. Biol. Chem. 267, 13425 (1992); Steinbeck et al., J. Biol. Chem. 268, 15649 (1993). Thus, these cells therefore offer both a non-photochemical, biological source of ¹O₂* and the antibodies capable of processing this substrate into reactive oxygen species.

Most areas of the body do not have access to photochemical energy. Hence, if neutrophils provide a cellular source of ¹O₂, an analysis of the oxidants expelled by antibody-coated neutrophils after activation could provide an indication as to whether ozone or H₂O₂ production by such antibodies may have a physiological relevance.

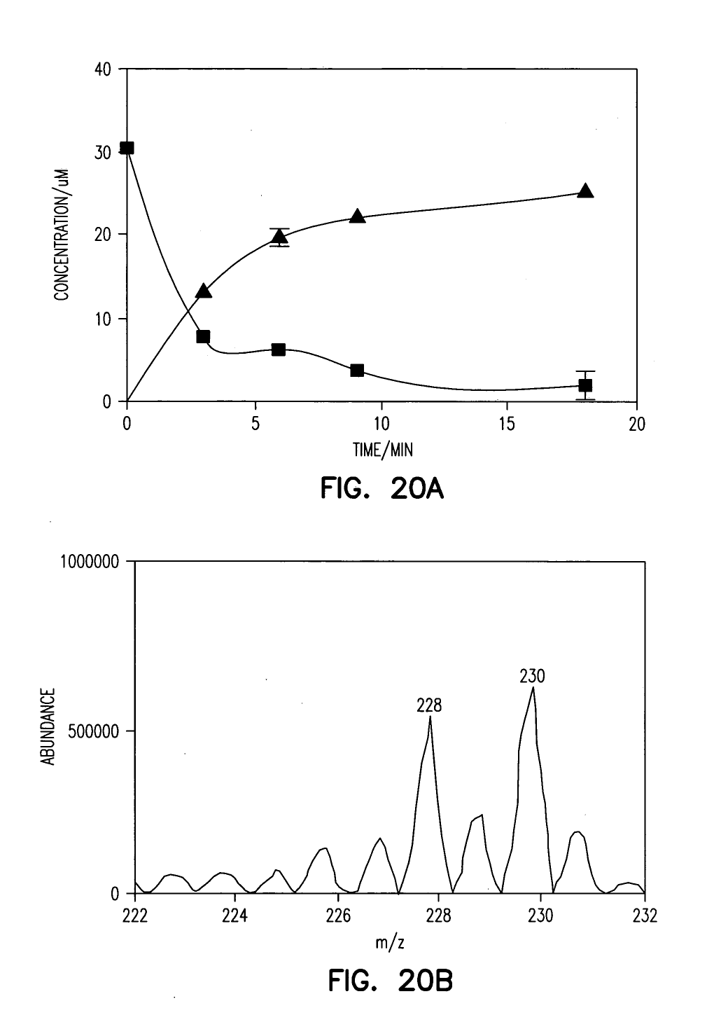

Human neutrophils were prepared as described by M. Markert, P. C. Andrews, and B. M. Babior Methods Enzymol. 105, 358 (1984). Following activation with phorbol myristate (1 μg/mL), the neutrophils (1.5×10⁷ cells/mL) produced an oxidant species that oxidatively cleaves indigo carmine 1 to isatin sulfonic acid 2 (FIG. 19 and FIG. 20B). Hypochlorous acid (HOCI) is an oxidant that is known to be produced by neutrophils. However, tests of NaOCl (2 mM) in PBS (pH 7.4) oxidized indigo carmine 1 (100 μM) but did not cleave the double bond of indigo carmine 1 to yield isatin sulfonic acid 2.

When the oxidation of indigo carmine 1 was carried out in ¹⁸O water, 50% of the lactam carbonyl oxygen was found to consist of ¹⁸O, as revealed by the intensity of the [M−H]-230 mass peak in the mass spectrum of the isolated cleaved product isatin sulfonic acid 2 (FIG. 20B). This ¹⁸O incorporation indicates that ozone was generated by the antibody-coated neutrophils.

FIG. 20A illustrates the time course of oxidation of indigo carmine 1 (30 μM) (Δ) and formation of isatin sulfonic acid 2 (▪) by human neutrophils (PMNs, 1.5×10⁷ cell/mL) that had been activated with phorbol myristate (1 μg/mL) in PBS (pH 7.4) at 37° C. Interestingly, almost 50% of the possible yield of isatin sulfonic acid 2 (25.1±0.3 μM of a potential 60 μM) from indigo carmine 1 was observed during the neutrophil cascade, revealing a significant concentration of the oxidant responsible for this transformation in the oxidative pathway.

### Publications

Allen, R. C., Stjernholm, R. L., Benerito, R. R. & Steele, R. H., eds. Cormier, M. J., Hercules, D. M. & Lee, J. (Plenum, New York), pp. 498-499 (1973).

Allen, R. C., Yevich, S. J., Orth, R. W. & Steele, R. H., Biochem. Biophys. Res. Commun., 60, 909-917 (1974).

Arlaud, G. J., Colomb, M. G. & Gagon, J., Immunol. Today, 8, 106-111 (1987).

Baek, J. & Kim, S., Plant Physiol., 102, 687 (1993).

Bent. D. V. & Hayon, E., J. Am. Chem. Soc., 87, 2612-2619 (1975).

Beauchamp, C. & Fridovich, I., Anal. Biochem., 44, 276-287 (1971).

F. Berthiaume, S. R. Reiken, M. Toner, R. G. Tomkins, M. L. Yarmush Biotechnology 12, 703 (1994).

G. M. Blackburn, A. Datta, H. Denham, P. Wentworth, Jr. Adv. Phys. Org. Chem.31, 249 (1998).

A. T. Brünger et al., Acta. Crystallogr., D54, 905 (1998)

Burley, S. K. & Petsko, G. A., Science, 229, 23-28 (1985).

Burton, D. R., Trends Biochem. Sci., 15, 64-69 (1990).

F. Cacace, G. de Petris, F. Pepi, A. Troiani, Science 285, 81 (1999).

V. Cannac-Caffrey, et al., Biochimie, 80, 1003 (1998).

J. Cerkovnik, B. Plesnicar, J. Am. Chem. Soc., 115, 12169 (1993).

E. J. Corey, Mehrotra, M. M.; Khan, A. U., J. Am. Chem. Soc., 108,2472 (1986).

C. Deby, La Recherche, 228, 378 (1991).

M. Detty, S. L. Gibson, J. Am. Chem. Soc., 112, 4086 (1990).

R. M. Esnouf, Acta Crystallog., D55, 938 (1999)].

Fee, J. A. in International Conference on Oxygen and Oxygen-Radicals, eds. Rodgers, M. A. J. & Powers, E. L. (Academic, San Diego, and University of Texas at Austin), pp. 205-239 (1981).

Feldhoff, R. & Peters, T. J., Biochem. J., 159, 529-533 (1976).

Foote, C. S. in Free Radicals in Biology, ed. Pryor, W. A. (Academic, New York), pp. 85-133 (1976).

C. S. Foote, Science, 162, 963 (1968).

C. S. Foote, Acc. Chem. Res., 1, 104 (1968).

A. V. Fowler, I. Zabin, J. Biol. Chem., 253, 5521 (1978).

K. C. Garcia et al., Science, 274, 209 (1996).

Gollnick, K., Adv. Photochem., 6, 1-122 (1968).

A. A. Gorman and M. A. J. Rodgers in Singlet Oxygen Chemistry, 205 (1988).

H. Greeley, T. V. Russo, D. T. Mainz, R. A. Friesner, J.-M. Langlois, W. A. Goddard III, R. E. Donnelly, J. Chem. Phys., 101, 4028 (1994)

J. C. Slater in Quantum Theory of Molecules and Solids, Vol. 4: The Self-Consistent Field of Molecules and Solids, McGraw Hill, New York, (1974)

Grossweiner, L. I., Curr. Top. Radiat. Res. Q., 11, 141-199 (1976).

J. Han, S. Yen, G. Han, P. Han, Anal. Biochem., 234, 107 (1996).

Hasty, N., Merkel, P. B., Radlick, P. & Kearns, D. R. Tetrahedron Lett., 49-52 (1972).

P. Hofman, M. Piche, D. F. Far, G. Le Negrate, E. Selva, L. Landraud, A. Alliana-Schmid, P. Boquet, B. Rossi, Infect. Immun. 68,449 (2000).

E. A. Kabat, T. T. Wu, H. M. Perry, K. S. Gottesman, C. Foeller, Sequences of Proteins of Immunological Interest (US Department of Health and Human Services, Public Health Service, NIH, ed. 5th, 1991).

J. R. Kanowsky, Chem. Biol. Interactions, 70, 1 (1989).

Kearns, D. R., Chem. Rev., 71, 395-427 (1971).

Klebanoff, S. J. in The Phagocytic Cell in Host Resistance (National Institute of Child Health and Human Development, Orlando, Fla.) (1974).

Klebanoff, S. J. in Encyclopedia of Immunology, eds. Delves, P. J. & Roitt, I. M. (Academic, San Diego), pp. 1713-1718 (1998).

J. Koller, B. Plesnicar, J. Am. Chem. Soc. 118, 2470 (1996).

Kreitner, M., Alth, G., Koren, H., Loew, S. & Ebermann, R., Anal. Biochem., 213, 63-67 (1993).

T. Li, S. Hilton, K. D. Janda, J. Am. Chem. Soc., 117, 3308 (1995).

D. R. Lide, in Handbook of Chemistry and Physics, 73rd ed. (CRC, 1992).

J. R. Kanofsky, H. Hoogland, R. Wever, S. J. Weiss J. Biol. Chem. 263, 9692 (1988).

J. F. Kanofsky Chem.-Biol. Interactions 70, 1 (1989)

Mach, H., Burke, C. J., Sanyal, G., Tsai, P.-K, Volkin, D. B. & Middaugh, C. R. in Formulation and Delivery of Proteins and Peptides, eds. Cleland, J. L. & Langer, R. (American Chemical Society, Denver, Colo.) (1994).

M. Markert, P. C. Andrews, and B. M. Babior Methods Enzymol. 105, 358 (1984).

A. C. R. Martin, PROTEINS: Struct., Funct. and Genet., 25, 130 (1996).

J. P. McCormick, T. Thomason, J. Am. Chem. Soc., 100, 312 (1978).

Merkel, P. B., Nillson, R. & Kearns, D. R., J. Am. Chem. Soc., 94, 1030-1031 (1972).

B. Michaeli, J. Feitelson, Photochem. Photobiol., 59, 284 (1994).

Petyaev, I. M. & Hunt, J. V., Redox Report, 2, 365-372 (1996).

Pierson, R., Young, V., Rees, J., Powell, J., Navaratnam, V., Cary, N., Tew, D., Bacon, P., Wallwork, J. et al., Microsc. Res. Tech., 42, 369-385 (1998).

B. Plesnicar, J. Cerkovnik, T. Tekavec, J. Koller, Chem. Eur. J., 6, 809 (2000).

T. Prange et al., PROTEINS: Struct., Funct. and Genet., 30, 61 (1998).

E. P. Reeves et al., Nature 416, 291 (2002).

D. T. Sawyer, in Oxygen Chemistry (Oxford University Press, Oxford, 1991).

H. D. Scharf, R. Weitz, Symp. Quantum Chem. Biochem., Jerusalem vol. 12 (Catal. Chem. Biochem.: Theory Exp.), pp. 355-365 (1979).

B. P. Schoenborn. H. C. Watson, J. C. Kendrew, Nature, 207, 28 (1965).

E. E. Scott, Q. H. Gibson, Biochemistry, 36, 11909 (1997).

L. Siegfried, M. Kmetove, H. Puzova, M. Molokacova, J. Filka, J. Med. Microbiol. 41, 127 (1994).

Sim, R. B. & Reid, K. B., Immunol. Today, 12, 307-311 (1991).

Skepper, J., Rosen, H. & Klebanoff, S. J., J. Biol. Chem., 252, 4803-4810 (1997).

S. M. Soltis, M. A. B. Stowell. M. C. Wiener, G. N. Phillips Jr, D. C. Rees, J. Appl. Cryst., 30, 190, (1997)

Srinivasan, V. S., Podolski, D., Westrick, N. J. & Neckers, D. C., J. Am. Chem. Soc., 15 100, 6513-6515 (1978).

Stauff, J., Sander, U. & Jaeschke, W., Chemiluminescence and Bioluminescence, eds., Williams, R. C. & Fudenberg, H. H. (Intercontinental Medical Book Corp., New York), pp. 131-141 (1973).

R. C. Straight, J. D. Spikes, in Singlet O₂, A. A. Frimer, Ed. (CRC Press, Inc., Boca Raton, Fla., 1985), vol. IV9, pp. 91-143.

M. J. Steinbeck, A. U. Khan, M. J. Karnovsky J. Biol. Chem. 267, 13425 (1992).

M. J. Steinbeck, A. U. Khan, M. J. Karnovsky J. Biol. Chem. 268, 15649 (1993).

K. Takeuchi, I. Takashi Anal. Chem. 61, 619 (1989).

K. Takeuchi, S. Kutsuna, T. Ibusuki Anal. Chim. Acta. 230, 183 (1990).

R. F. Tilson Jr., U. C. Singh, I. D. Kuntz Jr. P. A. Kollman, J. Mol. Biol., 199, 195 (1988).

M. A. Vincent, I. A. Hillier, J. Phys. Chem., 99, 3109 (1995).

Voss, R.-H., Ermler, U., Essen, L.-O., Wenzl, G., Kim, Y.-M. & Flecker, P., Eur. J. Biochem., 242, 122-131 (1996).

J. Wagner, R. A. Lemer, C. F. Barbas, III, Science 270, 1797 (1995).

P. Walrant, R. Santus, Photochem. Photobiol., 19, 411 (1974).

K. G. Welinder, H. M. Jespersen, J. W.-Rasmussen, K. Skoedt, Mol. Immunol., 28, 177 (1991).

F. Wilkinson, W. P. Helman, A. B. Ross, J. Phys. Chem. Ref. Data, 22, 113 (1993).

J. R. Winkler, A. J. Di Bilio, N. A. Farrow, J. H. Richards, H. B. Gray, Pure & Appl. Chem., 71, 1753 (1999).

J. R. Winkler, Curr. Opin. Chem. Biol., 4, 192 (2000).

Wentworth, P., Jr. & Janda, K. D., Curr. Opin. Chem. Biol., 2, 138-144 (1998).

A. D. Wentworth, L. H Jones, P. Wentworth, Jr., K. D. Janda, R. A. Lerner, Proc. Natl. Acad. Sci. U.S.A., 97, 10930 (2000).

P. Wentworth, Jr. et al Science 293, 1806 (2001).

P. Wentworth, Jr. Science 296, 2247 (2002).

X. Zhai and M. Ashraf Am. J. Physiol.269 (Heart Circ. Physiol. 38) H1229 (1995).

M. Zhou, Z. Diwu, N. Panchuk-Voloshina, R. P. Haugland, Anal. Biochem., 253, 162 (1997).

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A method for assaying for an immunological response in a mammal comprising: (a) administering to the mammal a chemical probe for reactive oxygen species; (b) obtaining a sample from the mammal; and (c) analyzing the sample for an oxidation product of the chemical probe.

2. The method of claim 1, wherein the chemical probe is an alkene that can be oxidized and that generates a detectable oxidation product.

3. The method of claim 1, wherein the chemical probe is 3-vinyl-benzoic acid, 4-vinyl-benzoic acid, indigo carmine, stilbene, or cholesterol.

4. The method of claim 1, wherein the reactive oxygen species is an antibody-generated oxygen species.

5. The method of claim 1, wherein the reactive oxygen species is a superoxide radical, hydroxyl radical, peroxyl radical or hydrogen peroxide.

6. The method of claim 1, wherein the reactive oxygen species is ozone or any chemical species that possesses the chemical signature of ozone.

7. The method of claim 1, wherein the sample is a bodily fluid.

8. The method of claim 7, wherein the bodily fluid is whole blood, serum, plasma, synovial fluid, lymph, urine, saliva, mucus or tears.

9. The method of claim 1, wherein the sample is a tissue sample.

10. The method of claim 1, wherein the oxidation product of the chemical probe is detected by high pressure liquid chromatography, mass spectrometry, ultraviolet light spectrophotometry, visible light spectrophotometry, liquid chromatography, gas spectrometry, or liquid chromatography linked mass spectrometry.

11. A method for assaying for an inflammatory response in a mammal comprising: (a) administering to the mammal a chemical probe for reactive oxygen species; (b) obtaining a sample from the mammal; and (c) analyzing the sample for an oxidation product of the chemical probe.

12. The method of claim 11, wherein the chemical probe is an alkene that can be oxidized and that generates a detectable oxidation product.

13. The method of claim 11, wherein the chemical probe is 3-vinyl-benzoic acid, 4-vinyl-benzoic acid, indigo carmine, stilbene, or cholesterol.

14. The method of claim 11, wherein the reactive oxygen species is an antibody-generated oxygen species.

15. The method of claim 11, wherein the reactive oxygen species is a superoxide radical, hydroxyl radical, peroxyl radical or hydrogen peroxide.

16. The method of claim 11, wherein the reactive oxygen species is ozone or a chemical species that possesses the chemical signature of ozone.

17. The method of claim 11, wherein the sample is a bodily fluid.

18. The method of claim 17, wherein the bodily fluid is whole blood, serum, plasma, synovial fluid, lymph, urine, saliva, mucus or tears.

19. The method of claim 11, wherein the sample is a tissue sample.

20. The method of claim 11, wherein the oxidation product of the chemical probe is detected by high pressure liquid chromatography, mass spectrometry, ultraviolet light spectrophotometry, visible light spectrophotometry, liquid chromatography, gas spectrometry, or liquid chromatography linked mass spectrometry.

21. An in vitro assay for neutrophil activity comprising: (a) obtaining a neutrophil sample from a mammal; (b) activating neutrophils in the neutrophil sample; and (c) observing whether a reactive oxygen species can be detected in the neutrophil sample.

22. The method of claim 21, wherein the reactive oxygen species is a neutrophil-generated oxygen species.

23. The method of claim 21, wherein the reactive oxygen species is an antibody-generated oxygen species.

24. The method of claim 21, wherein the reactive oxygen species is a superoxide radical, hydroxyl radical, peroxyl radical or hydrogen peroxide.

25. The method of claim 21, wherein the reactive oxygen species is ozone or a chemical species that possesses the chemical signature of ozone.

26. The method of claim 21, wherein the reactive oxygen species is detected with a chemical probe.

27. The method of claim 26, wherein the chemical probe is an alkene that can be oxidized and that generates a detectable oxidation product.

28. The method of claim 26, wherein the chemical probe is 3-vinyl-benzoic acid, 4-vinyl-benzoic acid, indigo carmine, stilbene, or cholesterol.

29. The method of claim 27, wherein an oxidation product of the chemical probe is detected in order to determine whether a reactive oxygen species is present in the neutrophil sample.

30. The method of claim 29, wherein the oxidation product is detected by high pressure liquid chromatography, mass spectrometry, ultraviolet light spectrophotometry, visible light spectrophotometry, liquid chromatography, gas spectrometry, or liquid chromatography linked mass spectrometry.

31. A method for identifying an agent that can modulate neutrophil activity comprising: (a) obtaining a neutrophil sample from a mammal; (b) exposing the neutrophil sample to a test agent; (c) activating neutrophils in the neutrophil sample; and (d) quantifying an amount of reactive oxygen species generated by the neutrophil sample.

32. The method of claim 31, wherein the method further comprises quantifying an amount of reactive oxygen species generated by a neutrophil sample that has not been exposed to the test agent but is from the same mammal.

33. The method of claim 31, wherein the neutrophil sample is a bodily fluid.

34. The method of claim 33, wherein the bodily fluid is whole blood, synovial fluid or lymph.

35. The method of claim 31, wherein the neutrophil sample is a tissue sample.

36. The method of claim 31, wherein the reactive oxygen species is a neutrophil-generated oxygen species.

37. The method of claim 31, wherein the reactive oxygen species is an antibody-generated oxygen species.

38. The method of claim 31, wherein the reactive oxygen species is a superoxide radical, hydroxyl radical, peroxyl radical or hydrogen peroxide.

39. The method of claim 31, wherein the reactive oxygen species is ozone or a chemical species that possesses the chemical signature of ozone.

40. The method of claim 31, wherein the amount of reactive oxygen species is quantified with a chemical probe.

41. The method of claim 40, wherein the chemical probe is an alkene that can be oxidized and that generates a detectable oxidation product.

42. The method of claim 40, wherein the chemical probe is 3-vinyl-benzoic acid, 4-vinyl-benzoic acid, indigo carmine, stilbene, or cholesterol.

43. The method of claim 40, wherein an oxidation product of the chemical probe is quantified.

44. The method of claim 43, wherein the oxidation product is quantified by high pressure liquid chromatography, mass spectrometry, ultraviolet light spectrophotometry, visible light spectrophotometry, liquid chromatography, gas spectrometry, or liquid chromatography linked mass spectrometry.
